(19) United States
(12) Patent Application Publication
(10) Pub. No.: US 2026/0007654 A1
(43) Pub. Date: Jan. 8, 2026

(54) METHODS OF TRICYCLIC AKR1C3 DEPENDENT KARS INHIBITOR DOSING FIELD OF THE INVENTION

(71) Applicant: NOVARTIS AG, Basel (CH)

(72) Inventors: Lisa Kattenhorn, Worcester, MA (US); Christy Fryer, Cambridge, MA (US); Heiko Maacke, Basel (CH); Margaret Elise McLaughlin, Cambridge, MA (US); Laurent L'Epicier-Sansregret, Basel (CH); Jeffrey Stonehouse, Littleton, MA (US)

(21) Appl. No.: 18/993,107

(22) PCT Filed: Jul. 18, 2023
(86) PCT No.: PCT/IB2023/057314

§ 371 Date: Jan. 10, 2025

Related U.S. Application Data
(60) Provisional application No. 63/369,399, filed on Jul. 26, 2022.

(51) Int. Cl. A61K 31/4747; A61P 35/00; G01N 33/573; G01N 33/574

(52) U.S. Cl. CPC A61K 31/4747; A61P 35/00; G01N 33/573; G01N 33/57488; G01N 2333/902

(57) ABSTRACT

The present invention relates to methods of identifying a subject for treatment with or treating a subject with a tricyclic AKR1C3 dependent KARS inhibitor of formula (I), or a pharmaceutically acceptable salt thereof. The methods may comprise determining in a subject sample a level of at least one of the following biomarkers: AKR1C3, NFE2L2, KEAP1, or CUL3, wherein an elevated level of the biomarker identifies the subject as being in need of treatment; or detecting in a subject sample a somatic mutation in at least one of the following genes: NFE2L2, KEAP1, or CUL3, wherein detecting the somatic mutation identifies the subject as being in need of treatment.

### FIELD OF THE INVENTION

The present invention relates to methods of identifying a subject for treatment with or treating a subject with a tricyclic Aldehyde keto reductase 1C3 (AKR1C3)-dependent lysyl-tRNA synthase (KARS) inhibitor of formula (I), or a pharmaceutically acceptable salt thereof. In embodiments described herein, the methods may comprise determining in a subject sample a level of a biomarker, for example, AKR1C3, wherein an elevated level of the biomarker identifies the subject as being in need of treatment; or detecting in a subject sample a somatic mutation in at least one of the following genes: NFE2 Like Bzip Transcription Factor 2 (NFE2L2), Kelch Like ECH Associated Protein 1 (KEAP1), or Cullin 3 (CUL3), wherein detecting the somatic mutation identifies the subject as being in need of treatment.

### BACKGROUND OF THE INVENTION

The NFE2L2/NFE2L2-KEAP1 pathway has a strong genetic basis in cancer. The Cancer Genome Atlas (TCGA) sequencing effort reported that this pathway was altered in 34% of lung squamous cell carcinomas (Hammerman et al., (2012)Nature 489:519-25). In addition, TCGA and other groups have reported significant mutation of this pathway in other solid tumor indications, including head and neck squamous cell carcinoma and hepatocellular carcinoma. Aberrant activation of the NFE2L2 pathway can occur by gain of function genetic alterations in NFE2L2 or loss of function genetic alterations in KEAP1 or CUL3 that lead to stabilization of NFE2L2 and elevated expression of its target genes. The uncontrolled transcription of those target genes confers advantages to cancer cells such as malignancy and protection against oxidative stress, chemotherapy and radiotherapy (Jaramillo and Zhang (2013) Genes Dev. 27:2179-91). Exacerbated NFE2L2 activity in tumors has been associated with poor prognosis (Shibata et al., (2008) Proc Natl Acad Sci USA 105:13568-73). There is currently no approved therapy to selectively target cancers with genetic alterations on the NFE2L2/KEAP1 pathway, which thus represents an unmet medical need.

Aldehyde Keto Reductase 1C3 (AKR1C3) is one of the numerous target genes of the transcription factor NFE2L2, whose expression is upregulated in NFE2L2/KEAP1 mutated cancers (MacLeod et al., (2016) Br J Cancer 115:1530-9). AKR1C3 (also named type 2 3α(17β)-hydroxysteroid dehydrogenase) is an NADP(H)-dependent ketosteroid reductase, a member of the aldo-keto reductase (AKR) superfamily, that plays a role in steroid hormone metabolism and signaling, as well as xenobiotic detoxification. Some known substrates for AKR1C3 are the endogenous substrates 5α-dihydrotestosterone, Δ4-androstene-3,17-dione and progesterone (Penning et al., (2000) Biochem. J. 351:67-77), as well as the synthetic prodrugs coumberone (Halim et al., (2008) J. Am. Chem. Soc. 130:14123-8), PR104 (Jamieson et al., (2014) Biochem Pharmacol. 88:36-45) and TH3424/OB1I3424 (International Publication No. WO 2016/145092). Tricyclic ketone compounds that get converted to lysine t-RNA synthetase (KARS) inhibitors by AKR1C3 in the presence of NADPH have also been identified.

AKR1C3 dependent KARS inhibitors provide an attractive strategy to selectively treat tumors that overexpress AKR1C3 compared to normal tissues, such as NFE2L2/KEAP1 mutated cancers and other types of cancers reported to overexpress AKR1C3 (Guise et al., (2010) Cancer Res. 70:1573-84) such as breast cancers (Lewis et al., (2004) BMC Cancer 4:27) and prostate cancers (Fung et al., (2006) Endocr Relat Cancer 13:169-80).

Compounds of formula (I), and pharmaceutically acceptable salts thereof, described in International Publication No. WO 2021/005586, have been identified as potent tricyclic AKR1C3-dependent KARS inhibitors. Currently, it remains a challenge to identify subjects for treatment with tricyclic AKR1C3-dependent KARS inhibitors and to determine a proper dose for treating such patients. There is a need to provide accurate and reliable methods for identifying and treating such patients.

### SUMMARY OF THE INVENTION

AKR1C3-dependent KARS inhibitors provide treatments and therapies for patients suffering from cancers with genetic alterations of the NFE2L2/KEAP1 pathway. The invention described herein provides methods of identifying a subject for treatment with an AKR1C3-dependent KARS inhibitor compound of formula (I), or a pharmaceutically acceptable salt thereof. The invention described herein also provides methods of selecting a compound of formula (I), or a pharmaceutically acceptable salt thereof, for treating a subject. The invention described herein also provides methods of treating a subject with a compound of formula (I), or a pharmaceutically acceptable salt thereof.

In some embodiments, the methods described herein comprise determining in a subject sample a level of a biomarker, for example, an AKR1C3 biomarker, for example, a level of AKR1C3 protein or a level of AKR1C3 mRNA. In some embodiments of the methods described herein, a subject sample is characterized as having an elevated level of AKR1C3, for example, an elevated level of AKR1C3 protein or an elevated level of AKR1C3 mRNA. In some embodiments, an elevated level of AKR1C3 identifies the subject as a subject in need of treatment with a compound of formula (I), or a pharmaceutically acceptable salt thereof.

In some embodiments, the methods described herein comprise detecting in a subject sample a somatic mutation in at least one of the following genes: NFE2L2, KEAP1, or CUL3. In some embodiments of the methods described herein, a subject sample is characterized by the presence of a somatic mutation in at least one of the following genes: NFE2L2, KEAP1, or CUL3. In some embodiments, the somatic mutation identifies the subject as in need of treatment with a compound of formula (I), or a pharmaceutically acceptable salt thereof.

In some embodiments, a method described herein comprises a step of administering a compound of formula (I), or a pharmaceutically acceptable salt thereof, to a subject. In some embodiments, the method comprises administering an amount, for example, an effective amount, for example, a therapeutically effective amount, of the compound of formula (I), or a pharmaceutically acceptable salt thereof, to the subject.

Various embodiments of the invention are described herein.

In one aspect of the invention, described herein is a method of identifying a subject for treatment with a compound of formula (I):

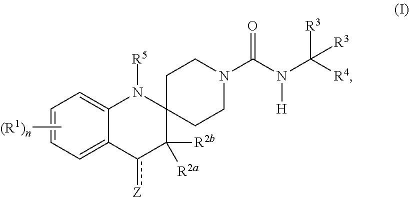

wherein [symbol] is a single bond or a double bond;
Z is either OH, when [symbol] is a single bond; or O, when [symbol] is a double bond;
each R¹ is independently selected from the group consisting of, (C₁-C₆)alkyl, (C₁-C₆)alkoxy, (C₀-C₄)alkylN(R⁸)₂, and halo;
R^{2a} and R^{2b} are each independently selected from the group consisting of H, (C₁-C₆) alkyl, and halo;
each R³ is independently selected from the group consisting of H, and halo;
R⁴ is selected from the group consisting of aryl, a 5 to 6-membered heteroaryl comprising 1, 2, 3, or 4 heteroatoms independently selected from N, O, and S; and a 9 to 10-membered fused bicyclic heteroaryl comprising 1, 2, 3, or 4 heteroatoms independently selected from N, O, and S; wherein any of the foregoing is optionally substituted with one or more R⁶
R⁵ is selected from the group consisting of H; (C₁-C₆)alkyl; (C₂-C₆)alkenyl; (C₀-C₄)alkylOR⁸; (C₁-C₄)alkyl(C₃-C₁₀)cycloalkyl; halo(C₁-C₆)alkyl; (C₂-C₃)alkynyl; (C₁-C₄)alkylN(R¹⁰)₂;
each R⁶ is independently selected from the group consisting of halo; (C₁-C₆)alkyl; (C₁-C₆)alkoxy; halo(C₁-C₆)alkyl; OH; aryl; 3 to 6-membered heterocycle; 5- to 6-membered heteroaryl; (C₀-C₄)alkylS(O)ₘ(C₁-C₆)alkyl; halo(C₁-C₆)alkoxy; (C₀-C₄)alkylS(O)ₘN(R⁸)₂; (C₀-C₄)alkyl N(R⁸)₂; (C₀-C₄)alkyl(CO)OR⁷; N(R⁸)S(O)ₘ(C₁-C₆)alkyl; N(R⁸)S(O)ₘ(C₃-C₆)cycloalkyl; OP(O)(OH)₂; (C₀-C₃)alkyl(CO)NHR¹¹; (C₀-C₃)alkylOR⁷, and (C₃-C₁₀)cycloalkyl; wherein each R⁶, when not being halo, OH, or OP(O)(OH)₂, is optionally substituted with one to three R⁹; or two neighboring R⁶, together with the atoms to which they attach form a 5 to 7-membered heterocycle or (C₅-C₈)cycloalkyl;
each R⁷ and R⁸ is independently selected from the group consisting of H or (C₁-C₆)alkyl, that is optionally substituted with one to three R⁹;
each R⁹ is independently selected from the group consisting of halo; —OH; amino, (C₁-C₄)alkylamino, di(C₁-C₄)alkylamino, OP(O)(OH)₂; (C₁-C₆)alkyl; (C₁-C₃)alkynyl; (C₁-C₆)alkoxy; halo(C₁-C₆)alkyl; (C₀-C₄)alkylS(O)ₘ(C₁-C₆)alkyl; halo(C₁-C₆)alkoxy; 3 to 6-membered heterocycle which is optionally substituted with oxo (═O); (C₀-C₄)alkylS(O)ₘN(R¹⁰)₂; (C₀-C₄)alkyl(CO)R¹⁰; (C₀-C₄)alkyl(CO)OR¹⁰; (C₀-C₄)alkylNR¹⁰S(O)ₘ(C₁-C₆)alkyl; (C₀-C₄)alkylOR¹⁰; (C₀-C₄)alkylN(R¹⁰)₂; (C₀-C₄)alkylCN; (C₀-C₄)alkylN(R¹⁰)₂; and (C₀-C₄)alkyl(CO)N(R¹⁰)₂;
each R¹⁰ is independently selected from the group consisting of H, (C₁-C₆)alkyl; or 3 to 6-membered heterocycle, wherein the 3 to 6-membered heterocycle is optionally substituted with one or more of (C₁-C₆)alkyl; and oxo (═O);
each R¹¹ is selected from the group consisting of H; 4 to 6-membered heterocycle which is optionally substituted with one to four R¹²; (C₃-C₆)cycloalkyl which is optionally substituted with one to four R¹²; (C₀-C₃)alkyl(C₃-C₆)cycloalkyl (C₁-C₃)alkyl which is optionally substituted with halo; CH₂-aryl which is optionally substituted with one to three R¹²; (C₁-C₆)alkyl; (C₂-C₆)alkenyl; or (C₂-C₆)alkynyl, wherein each of the (C₁-C₆)alkyl; (C₂-C₆)alkenyl; and (C₂-C₆)alkynyl is optionally substituted with one or more R¹³
each R¹² is independently selected from the group consisting of OH, (C₁-C₃)alkoxy, NH₂; or (C₁-C₃)alkyl optionally substituted with one or more OH;
each R¹³ is independently selected from the group consisting of halo, OH, amino, (C₁-C₄)alkylamino, di(C₁-C₄)alkylamino, (C₁-C₃)alkoxy; and C(O)—(C₃-C₈)cycloalkyl;
m is 0, 1, or 2; and
n is 0, 1 or 2,
   or a pharmaceutically acceptable salt thereof. In some embodiments, the method includes determining in a subject sample a level of AKR1C3. In some embodiments, an elevated level of AKR1C3 identifies the subject as a subject in need of treatment with the compound of formula (I), or a pharmaceutically acceptable salt thereof.

In another aspect of the invention, described herein is a method of selecting a compound of formula (I), or a pharmaceutically acceptable salt thereof, for treating a subject. In some embodiments, the method includes determining in a subject sample a level of AKR1C3. In some embodiments, an elevated level of AKR1C3 identifies the subject as a subject in need of treatment of the compound of formula (I), or a pharmaceutically acceptable salt thereof.

In yet another aspect of the invention, described herein is a method of treating a subject. In some embodiments, the method comprises: determining in a subject sample a level of AKR1C3, wherein an elevated level of AKR1C3 identifies the subject as in need of treatment with a compound of formula (I), or a pharmaceutically acceptable salt thereof; and administering an effective amount of the compound of formula (I), or a pharmaceutically acceptable salt thereof, to the subject.

In some embodiments, a method of treating a subject described herein, comprises administering to the subject an effective amount of a compound of formula (I), or a pharmaceutically acceptable salt thereof. For example, described herein is a method of treating a subject, the method comprising administering to the subject an effective amount of a compound of formula (I), or a pharmaceutically acceptable salt thereof, wherein prior to said administering, a subject sample is characterized as having an level of AKR1C3.

Also described herein is a method of treating a subject with a compound of formula (I), or a pharmaceutically acceptable salt thereof, wherein a subject sample is characterized as having an elevated level of AKR1C3.

In some embodiments of a method described herein, the compound of formula (I) is a specific compound of formula (I), or a pharmaceutically acceptable salt thereof. In some embodiments, the compound of formula (I) is selected from a subset of compounds of formula (I), or a pharmaceutically acceptable salt thereof. For example, in some embodiments, the compound of formula (I) is 6′-fluoro-N-(4-fluorobenzyl)-4′-oxo-3′,4′-dihydro-1′H-spiro[piperidine-4,2′-quinoline]-1-carboxamide), or a pharmaceutically acceptable salt thereof. In some embodiments, the compound of formula (I) is N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof. In some embodiments, the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof.

In some embodiments of a method described herein, a subject sample is characterized as having an elevated level of AKR1C3. In some embodiments of a method described herein, the method comprises determining in a subject sample a level of AKR1C3.

In various embodiments described herein, a sample, for example, a subject sample, is or comprises a cell, a cell population, a cell lysate, a tissue, or a fluid of a subject. In some embodiments a subject sample comprises the contents of a cell, a cell population, a cell lysate, a tissue, or a fluid of a subject, for example, the mRNA or protein content of the cell, cell population, cell lysate, tissue, or fluid of a subject. In some embodiments a subject sample comprises a subject genome, transcriptome, or proteome. In some embodiments a subject sample comprises a subject tumor genome, transcriptome, or proteome.

A subject sample can be obtained from a subject by any suitable means, for example, by biopsy or cell or tissue extraction. Similarly, a control sample can be obtained from a control subject by any suitable means.

In some embodiments, the subject sample is or comprises a cell, wherein the cell is a cancerous cell, for example, a tumor cell, for example, a lung cancer tumor cell, a non-small cell lung cancer tumor cell, a lung adenocarcinoma tumor cell, a lung squamous cell carcinoma cell, a bladder tumor cell, a cervical tumor cell, an esophageal tumor cell, a head and neck tumor cell, a kidney tumor cell, or a liver tumor cell. In some embodiments, the subject sample is or comprises a cell, wherein the cell is a lung cell, a bladder cell, a stomach cell, a prostate cell, an esophageal cell, a gastrointestinal cell, a lymphatic cell, a nervous system cell, an ovary cell, a cervical cell, a vaginal cell, a pancreatic cell, a throat cell, an esophageal cell, a kidney cell, a small intestine cell, a large intestine cell, a blood cell, a red blood cell, a white blood cell, a platelet cell, or a liver cell. In some embodiments, the subject sample is or comprises a cell, wherein the cell is a lung cell. In some embodiments, the subject sample is or comprises a cell, wherein the cell is a cell of the respiratory tract. For example, in some embodiments, the cell is or comprises a bronchial cell, a bronchiolar cell, an alveolar cell (for example, an alveolar type I cell, an alveolar type II cell, or an alveolar macrophage), an epithelial basement membrane cell, an endothelial cell, an airway epithelial cell (for example, a goblet cell, a ciliated cell, a clara cell, a neuroendocrine cell, a basal cell, an intermediate cell, a serous cell, a brush cell, an oncocyte, a nonciliated columnar cell, a metaplastic cell, a squamous cell, a clara-mucous cell, or a bronchiolar metaplasia cell), a salivary gland cell (for example, a salivary gland serous cell, mucous cell, or ductal cell), an interstitial connective tissue cell (for example, a smooth muscle cell, a cartilage cell, a fibroblast cell, a myofibroblast cell, a meningothelioid cell, an adipose tissue cell, or a neural cell), a blood vessel cell (for example, an endothelial cell, a smooth muscle cell, a fibroblast cell, a myofibroblast cell, a pericyte cell, a lymphatic cell, a hematopoietic cell, a lymphoid tissue cell (for example, a lymphocyte, a plasma cell, a megakaryocyte cell, a macrophage cell, a langerhans cell, a mast cell, an eosinophil cell, a neutrophil cell, or a basophil cell), a pleural cell (for example, a mesothelial cell, a pleuripotent submesothelial fibroblast cell, or an adipose cell), a stem cell, a perivascular epithelioid cell, a pluripotent epithelial stem cell, a meningothelioid cell, or an endothelial progenitor cell. In some embodiments, the subject sample is or comprises a fluid, wherein the fluid is selected from the group consisting of blood, plasma, mucus, urine, and lymphatic fluid. In some embodiments a subject sample comprises a subject genome, transcriptome, or proteome, for example, a subject tumor genome, transcriptome, or proteome, of any of the foregoing cells or fluids.

In some embodiments described herein, the subject is diagnosed with, in need of treatment for, being treated for, in remission of, at risk of developing, or predisposed to developing a disease or disorder, for example, a cancer. In some embodiments, the disease or disorder is selected from the group consisting of non-small cell lung cancer, lung adenocarcinoma, lung squamous cell carcinoma, a bladder cancer (for example, bladder urothelial carcinoma), a cervical cancer (for example, cervical squamous cell carcinoma), a uterine cancer (for example, uterine endometrial carcinoma), an esophageal cancer (for example, esophageal squamous cell carcinoma), a head and neck cancer (for example, head and neck squamous cell carcinoma), a kidney cancer (for example, papillary renal cell carcinoma), a breast cancer, colorectal cancer, a melanoma, a stomach cancer, castration-resistant prostate cancer (CRPC), T-cell acute lymphoblastic leukemia (T-ALL), acute myeloid leukemia (AML), myelodysplastic syndrome (MDS), and a liver cancer (for example, hepatocellular carcinoma). In some embodiments, the subject is diagnosed with a disease or disorder selected from the group consisting of non-small cell lung cancer, lung adenocarcinoma, lung squamous cell carcinoma, a bladder cancer (for example, bladder urothelial carcinoma), a cervical cancer (for example, cervical squamous cell carcinoma), a uterine cancer (for example, uterine endometrial carcinoma), an esophageal cancer (for example, esophageal squamous cell carcinoma), a head and neck cancer (for example, head and neck squamous cell carcinoma), a kidney cancer (for example, papillary renal cell carcinoma), a breast cancer, colorectal cancer, a melanoma, a stomach cancer, castration-resistant prostate cancer (CRPC), T-cell acute lymphoblastic leukemia (T-ALL), acute myeloid leukemia (AML), myelodysplastic syndrome (MDS), and a liver cancer (for example, hepatocellular carcinoma). In particular embodiments, the subject is diagnosed with the disease or disorder, for example, a cancer.

In some embodiments of a method described herein, a subject tumor genome comprises a somatic mutation associated with a disease or disorder, for example a cancer. For example, in some embodiments, the subject tumor genome comprises a somatic mutation in one or more of the NFE2L2, KEAP1, or CUL3 gene sequences. In some embodiments, the presence of a somatic mutation in the subject tumor genome in the NFE2L2, KEAP1, or CUL3 gene sequence can indicate that the subject is in need of treatment with a compound of formula (I), or a pharmaceutically acceptable salt thereof.

In some embodiments, the level of AKR1C3 detected or characterized in a subject sample is compared to a control level of AKR1C3. In some embodiments, the level of AKR1C3 in a subject sample is elevated relative to a control level of AKR1C3. An elevated level of AKR1C3 in a subject sample relative to a control level of AKR1C3 can indicate that the subject is in need of treatment with a compound of formula (I), or a pharmaceutically acceptable salt thereof.

In some embodiments, a control level comprises a level of AKR1C3 of a control sample or a control data set. For example, in some embodiments, the control level comprises a level of AKR1C3 of a control sample or a control data set, wherein the biomarker is the biomarker characterized or determined in a subject sample (for example, AKR1C3 protein or AKR1C3 mRNA).

In various embodiments described herein, a control sample is or comprises a non-cancerous cell of the subject, a non-cancerous cell population of the subject, a non-cancerous tissue of the subject, a non-cancerous fluid of the subject, a non-cancerous cell of a control subject, anon-cancerous cell population of a control subject, anon-cancerous tissue of a control subject, or a non-cancerous fluid of a control subject. In some embodiments, a control sample is or comprises the contents of a cell, for example, the protein or mRNA content of: a non-cancerous cell of the subject, a non-cancerous cell population of the subject, a non-cancerous tissue of the subject, a non-cancerous fluid of the subject, a non-cancerous cell of a control subject, anon-cancerous cell population of a control subject, anon-cancerous tissue of a control subject, or a non-cancerous fluid of a control subject. In some embodiments a control sample comprises a non-cancerous cell genome, transcriptome, or proteome, for example, a genome, transcriptome, or proteome of a non-cancerous cell of the subject, or a genome, transcriptome, or proteome of a non-cancerous cell of a control subject.

In some embodiments, a control data set comprises biomarker (for example, AKR1C3) level data from a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, or a combination thereof. In some embodiments, a control data set comprises biomarker (for example, AKR1C3) level data obtained from the contents of a cell, for example, the protein or mRNA content of: a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, or a combination thereof. In some embodiments a control data set comprises biomarker (for example, AKR1C3) level data from one or more non-cancerous cell genomes, transcriptomes, or proteomes, for example, genomes, transcriptomes, or proteomes of one or more non-cancerous cells of the subject or genomes, transcriptomes, or proteomes of one or more non-cancerous cells of a control subject.

In some embodiments, the control sample is or comprises a cell, wherein the cell is a lung cell, a bladder cell, a stomach cell, a prostate cell, an esophageal cell, a gastrointestinal cell, a lymphatic cell, a nervous system cell, an ovary cell, a cervical cell, a vaginal cell, a pancreatic cell, a throat cell, an esophageal cell, a kidney cell, a small intestine cell, a large intestine cell, a blood cell, a red blood cell, a white blood cell, a platelet cell, or a liver cell. In some embodiments, the control sample is or comprises a cell, wherein the cell is a lung cell. In some embodiments, the control sample is or comprises a cell, wherein the cell is a cell of the respiratory tract. For example, in some embodiments, the cell is or comprises a bronchial cell, a bronchiolar cell, an alveolar cell (for example, an alveolar type I cell, an alveolar type II cell, or an alveolar macrophage), an epithelial basement membrane cell, an endothelial cell, an airway epithelial cell (for example, a goblet cell, a ciliated cell, a clara cell, a neuroendocrine cell, a basal cell, an intermediate cell, a serous cell, a brush cell, an oncocyte, a nonciliated columnar cell, a metaplastic cell, a squamous cell, a clara-mucous cell, or a bronchiolar metaplasia cell), a salivary gland cell (for example, a salivary gland serous cell, mucous cell, or ductal cell), an interstitial connective tissue cell (for example, a smooth muscle cell, a cartilage cell, a fibroblast cell, a myofibroblast cell, a meningothelioid cell, an adipose tissue cell, or a neural cell), a blood vessel cell (for example, an endothelial cell, a smooth muscle cell, a fibroblast cell, a myofibroblast cell, a pericyte cell, a lymphatic cell, a hematopoietic cell, a lymphoid tissue cell (for example, a lymphocyte, a plasma cell, a megakaryocyte cell, a macrophage cell, a langerhans cell, a mast cell, an eosinophil cell, a neutrophil cell, or a basophil cell), a pleural cell (for example, a mesothelial cell, a pleuripotent submesothelial fibroblast cell, or an adipose cell), a stem cell, a perivascular epithelioid cell, a pluripotent epithelial stem cell, a meningothelioid cell, or an endothelial progenitor cell. In some embodiments, the control sample is or comprises a fluid, wherein the fluid is selected from the group consisting of blood, plasma, mucus, urine, and lymphatic fluid. In some embodiments a control sample comprises a non-cancerous cell genome, transcriptome, or proteome (for example, a genome, transcriptome, or proteome of a non-cancerous cell of a subject or a genome, transcriptome, or proteome of a non-cancerous cell of a control subject) of any of the foregoing cells or fluids.

In some embodiments, a control data set comprises biomarker level data from a cell, wherein the cell is a lung cell, a bladder cell, a stomach cell, a prostate cell, an esophageal cell, a gastrointestinal cell, a lymphatic cell, a nervous system cell, an ovary cell, a cervical cell, a vaginal cell, a pancreatic cell, a throat cell, an esophageal cell, a kidney cell, a small intestine cell, a large intestine cell, a blood cell, a red blood cell, a white blood cell, a platelet cell, or a liver cell. In some embodiments, the control data set comprises biomarker level data from a cell, wherein the cell is a lung cell. In some embodiments, the control data set comprises biomarker (for example, AKR1C3) level data from a cell, wherein the cell is a cell of the respiratory tract. For example, in some embodiments, the cell is or comprises a bronchial cell, a bronchiolar cell, an alveolar cell (for example, an alveolar type I cell, an alveolar type II cell, or an alveolar macrophage), an epithelial basement membrane cell, an endothelial cell, an airway epithelial cell (for example, a goblet cell, a ciliated cell, a clara cell, a neuroendocrine cell, a basal cell, an intermediate cell, a serous cell, a brush cell, an oncocyte, a nonciliated columnar cell, a metaplastic cell, a squamous cell, a clara-mucous cell, or a bronchiolar metaplasia cell), a salivary gland cell (for example, a salivary gland serous cell, mucous cell, or ductal cell), an interstitial connective tissue cell (for example, a smooth muscle cell, a cartilage cell, a fibroblast cell, a myofibroblast cell, a meningothelioid cell, an adipose tissue cell, or a neural cell), a blood vessel cell (for example, an endothelial cell, a smooth muscle cell, a fibroblast cell, a myofibroblast cell, a pericyte cell, a lymphatic cell, a hematopoietic cell, a lymphoid tissue cell (for example, a lymphocyte, a plasma cell, a megakaryocyte cell, a macrophage cell, a langerhans cell, a mast cell, an eosinophil cell, a neutrophil cell, or a basophil cell), a pleural cell (for example, a mesothelial cell, a pleuripotent submesothelial fibroblast cell, or an adipose cell), a stem cell, a perivascular epithelioid cell, a pluripotent epithelial stem cell, a meningothelioid cell, or an endothelial progenitor cell. In some embodiments, the control data set comprises biomarker level data from a fluid, wherein the fluid is selected from the group consisting of blood, plasma, mucus, urine, and lymphatic fluid. In some embodiments a control data set comprises biomarker (for example, AKR1C3) level data from one or more non-cancerous cell genomes, transcriptomes, or proteomes (for example, genomes, transcriptomes, or proteomes of one or more non-cancerous cells of the subject, or genomes, transcriptomes, or proteomes of one or more non-cancerous cells of a control subject), wherein the biomarker level data is from one or more of the foregoing cell types and fluids.

In some embodiments, the subject sample and the control sample comprise the same type of cell, cell population, cell lysate, tissue, or fluid (or the protein or mRNA content thereof). In some embodiments, the subject sample AKR1C3 level is determined or characterized in a cell, a cell population, a cell lysate, a tissue, or a fluid (or from the protein or mRNA content thereof), and the control level of ARK1C3 is determined or characterized from a control data set comprising biomarker level data from the same or comparable type of cell, cell population, cell lysate, tissue, or fluid (or from the protein or mRNA content thereof). For example, in some embodiments, the subject sample comprises a lung cell and the control sample comprises a lung cell. For example, in some embodiments, the subject sample comprises a lung cell and the control data set comprises biomarker (for example, AKR1C3) level data from a lung cell. For example, in some embodiments, the subject sample comprises the genome, transcriptome, or proteome of a lung cell and the control sample comprises the genome, transcriptome, or proteome of a lung cell. For example, in some embodiments, the subject sample comprises the genome, transcriptome, or proteome of a lung cell and the control data set comprises biomarker (for example, AKR1C3) level data from the genome, transcriptome, or proteome of a lung cell.

In a method described herein, AKR1C3 biomarker data can be protein or nucleic acid biomarker data. For example, in some embodiments, the level of a biomarker is a protein level, a ribonucleic acid (for example, messenger ribonucleic acid (mRNA)) level, or a level of another suitable species of a biomarker.

In embodiments described herein, the level of AKR1C3 in a subject sample relative to a control level of AKR1C3 (for example, a level of AKR1C3 in a control sample or a control data set) indicates that the subject is in need of treatment with a compound of formula (I), or a pharmaceutically acceptable salt thereof. For example, in some embodiments, if the level of AKR1C3 in the subject sample is about 1.5 times greater, about 2 times greater, about 3 times greater, about 4 times greater, about 5 times greater, about 6 times greater, about 7 times greater, about 8 times greater, about 9 times greater, about 10 times greater, about 20 times greater, about 30 times greater, about 40 times greater, about 50 times greater, about 60 times greater, about 70 times greater, about 80 times greater, about 90 times greater, about 100 times greater, about 200 times greater, about 300 times greater, about 400 times greater, about 500 times greater, about 600 times greater, about 700 times greater, about 800 times greater, about 900 times greater, about 1000 times greater, about 1500 times greater, or about 2000 times greater than the level of AKR1C3 in the control sample or the control data set, then the subject is in need of treatment with the compound of formula (I), or a pharmaceutically acceptable salt thereof.

In some embodiments, if the level of AKR1C3 in the subject sample is at least about 1.5 times greater, at least about 2 times greater, at least about 3 times greater, at least about 4 times greater, at least about 5 times greater, at least about 6 times greater, at least about 7 times greater, at least about 8 times greater, at least about 9 times greater, at least about 10 times greater, at least about 20 times greater, at least about 30 times greater, at least about 40 times greater, at least about 50 times greater, at least about 60 times greater, at least about 70 times greater, at least about 80 times greater, at least about 90 times greater, at least about 100 times greater, at least about 200 times greater, at least about 300 times greater, at least about 400 times greater, at least about 500 times greater, at least about 600 times greater, at least about 700 times greater, at least about 800 times greater, at least about 900 times greater, at least about 1000 times greater, at least about 1500 times greater, or at least about 2000 times greater than the level of AKR1C3 in the control sample or the control data set, then the subject is in need of treatment with the compound of formula (I), or a pharmaceutically acceptable salt thereof.

In some embodiments, the level of AKR1C3 is determined or characterized by a specific assay. In some embodiments, determining the level of AKR1C3 comprises performing an antigen detection assay. For example, in some embodiments, the antigen detection assay is selected from the group consisting of a western blot assay, an enzyme-linked immunosorbent assay (ELISA), an immunohistochemistry (IHC) assay, an immunocytochemistry assay, a flow cytometry assay, an immunoprecipitation assay, an immuno-electrophoresis assay, and an immuno-electron microscopy assay. In particular embodiments, the antigen detection assay is an IHC assay.

In some embodiments, performing the antigen detection assay comprises probing the subject sample with an AKR1C3 antibody. The AKR1C3 antibody can be an anti-AKR1C3 mouse monoclonal antibody, clone NP6.G6.A6 (Cat. No. ab49680; Abcam, Waltham, MA). Examples of AKR1C3 antibodies suitable for use with the methods described herein include anti-AKR1C3 mouse monoclonal antibody, clone NP6.G6.A6; mouse monoclonal antibody, clone 871701 (Cat. No. MAB7678; R&D Systems, Inc., Minneapolis, MN); anti-AKR1C3 rabbit polyclonal antibody ab84327 (Cat. No. ab84327; Abcam, Waltham, MA); anti-AKR1C3 rabbit polyclonal antibody 11194-1-AP (Cat. No. 11194-1-AP; Thermo Fisher Scientific, Waltham, MA); anti-AKR1C3 rabbit polyclonal antibody PA5-106891 (Cat. No. PA5-106891; Thermo Fisher Scientific, Waltham, MA); anti-AKR1C3 rabbit polyclonal antibody PA5-97446 (Cat. No. PA5-97446; Thermo Fisher Scientific, Waltham, MA); anti-AKR1C3 rabbit polyclonal antibody PA5-29779 (Cat. No. PA5-29779; Thermo Fisher Scientific, Waltham, MA); anti-AKR1C3 rabbit polyclonal antibody PA5-76071 (Cat. No. PA5-76071; Thermo Fisher Scientific, Waltham, MA); anti-AKR1C3 goat polyclonal antibody PA5-18339 (Cat. No. PA5-18339; Thermo Fisher Scientific, Waltham, MA); anti-AKR1C3 rabbit polyclonal antibody BS-11401R (Cat. No. BS-11401R; Thermo Fisher Scientific, Waltham, MA); and anti-AKR1C3 rabbit polyclonal antibody NBP1-33556 (Cat. No. NBP1-33556; Novus Biologicals, Centennial, CO). In some embodiments, the AKR1C3 antibody comprises CDR sequences sharing at least 80% identity, at least 85% identity, at least 90% identity, at least 95% identity, at least 96% identity, at least 97% identity, at least 98% identity, or at least 99% identity with the CDR sequences of an anti-AKR1C3 antibody, for example, an anti-AKR1C3 mouse monoclonal antibody, for example, anti-AKR1C3 mouse monoclonal antibody clone NP6.G6.A6.

An AKR1C3 antibody used in a method described herein can be conjugated to an element that allows antibody detection using a detection assay. For example, in some embodiments, the AKR1C3 antibody is conjugated to horse radish peroxidase (HRP). In some embodiments, the AKR1C3 antibody is not conjugated to HRP or another element that allows antibody detection using a detection assay. In such embodiments, performing the antigen detection assay further comprises probing the subject sample with a secondary antibody, for example, a secondary antibody conjugated to HRP, or a probe capable of binding to the primary antibody, for example, streptavidin. The presence of HRP can be detected through a reaction with 3,3′-diaminobenzidine (DAB). Thus, in some embodiments (for example, embodiments where a primary antibody or a secondary antibody is conjugated to HRP), the antigen detection assay further comprises applying 3,3′-diaminobenzidine (DAB) to the subject sample.

In some embodiments of a method described herein, determining a AKR1C3 level, for example, a AKR1C3 level of a subject sample or a control sample, can include producing a signal intensity score. Thus, in some embodiments that include performing an IHC assay, determining a AKR1C3 level of a subject sample further comprises producing an IHC signal intensity score for the subject sample. In some embodiments that include performing an IHC assay, determining a AKR1C3 level of a control sample further comprises producing an IHC signal intensity score for the control sample. The IHC signal intensity score of a subject sample or the increase in the IHC signal intensity score of a subject sample relative to the IHC signal intensity score of a control sample can indicate that a subject is in need of treatment. For example, in some embodiments, if the IHC signal intensity score for the subject sample is 0.5 or greater, 1.0 or greater, 1.5 or greater, 2 or greater, 2.5 or greater, 2.6 or greater, 2.7 or greater, 2.8 or greater, or 2.9 or greater, and the IHC signal intensity score can range from 0-3, then the subject is in need of treatment with the compound of formula (I), or a pharmaceutically acceptable salt thereof. In some embodiments, if the IHC signal intensity score for the subject sample is 50 or greater, 100 or greater, 150 or greater, 200 or greater, 250 or greater, 260 or greater, 270 or greater, 280 or greater, or 290 or greater, and the IHC signal intensity score can range from 0-300, then the subject is in need of treatment with the compound of formula (I), or a pharmaceutically acceptable salt thereof.

In some embodiments of a method described herein, the subject sample is characterized as having an elevated AKR1C3 level by an antigen detection assay, for example, a western blot assay, an enzyme-linked immunosorbent assay (ELISA), an immunohistochemistry (IHC) assay, an immunocytochemistry assay, a flow cytometry assay, an immunoprecipitation assay, an immuno-electrophoresis assay, or an immuno-electron microscopy assay. In particular embodiments, the antigen detection assay is an IHC assay.

In further embodiments of a method described herein, determining the level of AKR1C3 (for example, the level of AKR1C3 mRNA) in a sample, for example a subject sample or a control sample) comprises performing a polymerase chain reaction (PCR). In such embodiments, the PCR is effective to determine the AKR1C3 level in a subject sample and/or a control sample. In some embodiments, determining a AKR1C3 level further comprises performing a PCR effective to determine the level of a control marker in the subject sample and/or the control sample. The control marker can be, for example, beta actin or glyceraldehyde-3-phosphate dehydrogenase (GAPDH). In some embodiments, the PCR is a quantitative PCR (qPCR), a reverse transcription PCR (RT-PCR), or a reverse transcription qPCR (RT-qPCR).

In some embodiments of a method described herein, the level of AKR1C3 is characterized in a sample (for example, a subject sample and/or a control sample) by a PCR. For example, in some embodiments, a subject sample is characterized as having an elevated AKR1C3 level (for example, an elevated AKR1C3 mRNA level) by a PCR, for example, a PCR effective to determine the AKR1C3 level in the subject sample. In some embodiments, the subject sample is characterized as having an elevated AKR1C3 level relative to the AKR1C3 level of a control sample or a control data set, for example, as determined by a PCR. Thus, in some embodiments, the level of AKR1C3 is determined in a control sample by a PCR effective to determine the AKR1C3 level in the control sample. In some embodiments, a level of a control marker is determined in a subject sample by a PCR effective to determine the control marker level in the subject sample. In some embodiments, a level of the control marker is determined in the control sample by a PCR effective to determine the control marker level in the control sample. The control marker can be, for example, beta actin or glyceraldehyde-3-phosphate dehydrogenase (GAPDH). In some embodiments, the PCR is a quantitative PCR (qPCR).

In another aspect of the invention, described herein is a method of identifying a subject in need of treatment with a compound of formula (I), or a pharmaceutically acceptable salt thereof, where the method comprises detecting in a subject sample a somatic mutation in at least one of the following genes: NFE2L2, KEAP1, or CUL3. In some embodiments, the somatic mutation is detected in NFE2L2. In some embodiments, the somatic mutation is detected in KEAP1. In some embodiments, the somatic mutation is detected in CUL3. In some embodiments, a somatic mutation is detected in each of NFE2L2 and KEAP1. In some embodiments, a somatic mutation is detected in each of NFE2L2 and CUL3. In some embodiments, a somatic mutation is detected in each of KEAP1 and CUL3. In some embodiments, a somatic mutation is detected in each of NFE2L2, KEAP1, and CUL3.

Also described herein is a method of selecting a compound of formula (I), or a pharmaceutically acceptable salt thereof, for treating a subject, the method comprising detecting in a subject sample a somatic mutation in at least one of the following genes: NFE2L2, KEAP1, or CUL3. In some embodiments, the somatic mutation is detected in NFE2L2. In some embodiments, the somatic mutation is detected in KEAP1. In some embodiments, the somatic mutation is detected in CUL3. In some embodiments, a somatic mutation is detected in each of NFE2L2 and KEAP1. In some embodiments, a somatic mutation is detected in each of NFE2L2 and CUL3. In some embodiments, a somatic mutation is detected in each of KEAP1 and CUL3. In some embodiments, a somatic mutation is detected in each of NFE2L2, KEAP1, and CUL3.

Also described herein is a method of treating a subject, the method comprising: detecting in a subject sample a somatic mutation in at least one of the following genes: NFE2L2, KEAP1, or CUL3; and administering an effective amount (for example, a therapeutically effective amount) of the compound of formula (I), or a pharmaceutically acceptable salt thereof, to the subject. In some embodiments, the detecting a somatic mutation identifies the subject as in need of treatment with a compound of formula (I), or a pharmaceutically acceptable salt thereof. In some embodiments, the somatic mutation is detected in NFE2L2. In some embodiments, the somatic mutation is detected in KEAP1. In some embodiments, the somatic mutation is detected in CUL3. In some embodiments, a somatic mutation is detected in each of NFE2L2 and KEAP1. In some embodiments, a somatic mutation is detected in each of NFE2L2 and CUL3. In some embodiments, a somatic mutation is detected in each of KEAP1 and CUL3. In some embodiments, a somatic mutation is detected in each of NFE2L2, KEAP1, and CUL3.

Also described herein is a method of treating a subject, the method comprising administering to the subject an effective amount of a compound of formula (I), or a pharmaceutically acceptable salt thereof, wherein prior to said administering, a subject sample is characterized by the presence of a somatic mutation in at least one of the following genes: NFE2L2, KEAP1, or CUL3. In some embodiments, the somatic mutation is in NFE2L2. In some embodiments, the somatic mutation is in KEAP1. In some embodiments, the somatic mutation is in CUL3. In some embodiments, a somatic mutation is in each of NFE2L2 and KEAP1. In some embodiments, a somatic mutation is in each of NFE2L2 and CUL3. In some embodiments, a somatic mutation is in each of KEAP1 and CUL3. In some embodiments, a somatic mutation is in each of NFE2L2, KEAP1, and CUL3.

Also described herein is a method of treating a subject with a compound of formula (I), or a pharmaceutically acceptable salt thereof, wherein a subject sample is characterized by the presence of a somatic mutation in at least one of the following genes: NFE2L2, KEAP1, or CUL3. In some embodiments, the somatic mutation is in NFE2L2. In some embodiments, the somatic mutation is in KEAP1. In some embodiments, the somatic mutation is in CUL3. In some embodiments, a somatic mutation is in each of NFE2L2 and KEAP1. In some embodiments, a somatic mutation is in each of NFE2L2 and CUL3. In some embodiments, a somatic mutation is in each of KEAP1 and CUL3. In some embodiments, a somatic mutation is in each of NFE2L2, KEAP1, and CUL3.

In some embodiments of a method described herein, the compound of formula (I) is a specific compound of formula (I), or a pharmaceutically acceptable salt thereof. In some embodiments, the compound of formula (I) is selected from a subset of compounds of formula (I), or a pharmaceutically acceptable salt thereof. For example, in some embodiments, the compound of formula (I) is 6′-fluoro-N-(4-fluorobenzyl)-4′-oxo-3′,4′-dihydro-1′H-spiro[piperidine-4,2′-quinoline]-1-carboxamide), or a pharmaceutically acceptable salt thereof. In some embodiments, the compound of formula (I) is N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof. In some embodiments, the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof.

In some embodiments of a method described herein, the somatic mutation is a disease-linked nucleotide gene sequence associated with the KEAP1, CUL3, NFE2L2, or AKR1C3 gene sequence. Somatic mutations and disease-linked nucleotide sequences associated with KEAP1, CUL3, NFE2L2, or AKR1C3 gene sequences include nucleotide sequences associated with a cancer, for example, lung cancer, for example, NSCLC. In some embodiments, the somatic mutation is a disease-linked mutation indicative of an increased likelihood of an individual carrying the somatic mutation of having, developing, or being predisposed to developing a particular disease. For example, an individual carrying a somatic mutation can have an increased likelihood of having, developing, or being predisposed to developing a cancer. In some embodiments, a subject tumor genome comprising a somatic mutation is indicative of an increased likelihood of the subject having, developing, or being predisposed to developing a cancer.

Somatic mutations and disease-linked nucleotide sequences associated with KEAP1 are described, for example, in Campbell et al., (2016) Nature Genetics, 48:607-16; Chen, R. (2020) “Cullin 3 and Its Role in Tumorigenesis” in Cullin-RING Ligases and Protein Neddylation, 187-210; Collisson et al., (2014) Nature, 511:543-550; Delgobo et al., (2021) Freed Radical Biology and Medicine, 177:58-71; Gong et al., (2020) Cell Communication and Signaling, 18, 98; Hammerman et al., (2012) Nature, 489:519-525; Hayes and McMahon (2009) Trends in Biochemical Sciences, 34(4):176-88; Jin et al., (2021) Cancer Medicine, 10(23):8673-92; Kandoth et al., (2013) Nature, 502:333-339; Konstantinopoulos et al. (2011) Cancer Res, 71:5081-5089; Ohta et al. (2008) Cancer Res, 68:1303-1309; Padmanabhan et al., (2006) Mol Cell, 21:689-700; Romero et al., (2020) Nature Cancer, 1(6):589-602; Saleh et al., (2021) Journal of Thoracic Oncology, 17(1): 76-88; Shibata et al., (2008) Gastroenterology, 135:1358-1368; Shibata et al. (2011) Neoplasia, 13:864-873; Singh et al., (2006) PLoS Med, 3:e420; Taguchi and Yamamoto (2017) Frontiers in Oncology, 7:85; Wang et al., (2020) “CRL3s: The BTB-CUL3-RING E3 Ubiquitin Ligases” in Cullin-RING Ligases and Protein Neddylation, 211-223; and Yoo et al., (2012) Histopathology, 60:943-952, which are incorporated by reference herein.

Somatic mutations and disease-linked nucleotide sequences associated with CUL3 are described, for example, in Campbell et al., (2016) Nature Genetics, 48:607-16; Chen, R. (2020) “Cullin 3 and Its Role in Tumorigenesis” in Cullin-RING Ligases and Protein Neddylation, 187-210; Collisson et al., (2014) Nature, 511:543-550; Delgobo et al., (2021) Freed Radical Biology and Medicine, 177:58-71; Hammerman et al., (2012) Nature, 489:519-25; Jin et al., (2021) Cancer Medicine, 10(23):8673-92; Ooi et al., (2013) Cancer Res, 73:2044-51; and Wang et al., (2020) “CRL3s: The BTB-CUL3-RING E3 Ubiquitin Ligases” in Cullin-RING Ligases and Protein Neddylation, 211-23, which are incorporated by reference herein.

Somatic mutations and disease-linked nucleotide sequences associated with NFE2L2 are described, for example, in Campbell et al., (2016) Nature Genetics, 48:607-16; Chen, R. (2020) “Cullin 3 and Its Role in Tumorigenesis” in Cullin-RING Ligases and Protein Neddylation, 187-210; Collisson et al., (2014) Nature, 511:543-50; Delgobo et al., (2021) Freed Radical Biology and Medicine, 177:58-71; Goldstein et al., (2016) Cell Rep, 16:2605-2617; Hammerman et al., (2012) Nature, 489:519-25; Jin et al., (2021) Cancer Medicine, 10(23):8673-92; Ooi et al., (2013) Cancer Res, 73:2044-51; Shibata et al., (2011) Neoplasia, 13:864-873; and Wang et al., (2020) “CRL3s: The BTB-CUL3-RING E3 Ubiquitin Ligases” in Cullin-RING Ligases and Protein Neddylation, 211-23, which are incorporated by reference herein.

A somatic mutation can include a mutation relative to the wild-type nucleotide sequence of a gene, for example, NFE2L2, KEAP1, or CUL3. In some embodiments, the somatic mutation comprises a mutation selected from the group consisting of a nonsense mutation, a missense mutation, a substitution mutation, a frameshift mutation, a point mutation, an insertion mutation, a deletion mutation (for example, a gene sequence deletion), an amplification mutation (for example, a gene amplification), an inversion mutation, and a duplication mutation. For example, in some embodiments, the somatic mutation comprises a mutation in the wild-type nucleotide sequence of NFE2L2, KEAP1, or CUL3, wherein the mutation is a nonsense mutation, a missense mutation, a substitution mutation, a frameshift mutation, a point mutation, an insertion mutation, a deletion mutation, an inversion mutation, or a duplication mutation. In some embodiments, the somatic mutation comprises a single nucleotide polymorphism (SNP), for example, a SNP of the NFE2L2, KEAP1, or CUL3 gene. In some embodiments, the somatic mutation is a mutation of a tumor cell genome, for example, a subject tumor cell genome.

In various embodiments described herein, a sample, for example, a subject sample, is or comprises a cell, a cell population, a cell lysate, a tissue, or a fluid of a subject. In some embodiments, a subject sample, is or comprises nucleic acid, for example, genomic deoxyribonucleic acid (DNA) or ribonucleic acid (RNA) (for example, messenger RNA (mRNA)), of a cell, a cell population, a cell lysate, a tissue, or a fluid of a subject. In some embodiments, a subject sample, is or comprises a cell genome or transcriptome, for example, a tumor cell genome or transcriptome.

A subject sample can be obtained from a subject by any suitable means. Similarly, a control sample can be obtained from a control subject by any suitable means.

In some embodiments, the subject sample is or comprises a cell (or a species of nucleic acid, for example, genomic DNA or mRNA, thereof), wherein the cell is a cancerous cell, for example, a tumor cell, for example, a lung cancer tumor cell, a non-small cell lung cancer tumor cell, a lung adenocarcinoma tumor cell, a lung squamous cell carcinoma cell, a bladder tumor cell, a cervical tumor cell, an esophageal tumor cell, a head and neck tumor cell, a kidney tumor cell, or a liver tumor cell. In some embodiments, the subject sample is or comprises a cell (or a species of nucleic acid, for example, genomic DNA or mRNA, thereof), wherein the cell is a lung cell, a bladder cell, a stomach cell, a prostate cell, an esophageal cell, a gastrointestinal cell, a lymphatic cell, a nervous system cell, an ovary cell, a cervical cell, a vaginal cell, a pancreatic cell, a throat cell, an esophageal cell, a kidney cell, a small intestine cell, a large intestine cell, a blood cell, a red blood cell, a white blood cell, a platelet cell, or a liver cell. In some embodiments, the subject sample is or comprises a cell (or a species of nucleic acid, for example, genomic DNA or mRNA, thereof), wherein the cell is a lung cell. In some embodiments, the subject sample is or comprises a cell (or a species of nucleic acid, for example, genomic DNA or mRNA, thereof), wherein the cell is a cell of the respiratory tract. For example, in some embodiments, the cell is or comprises one of the following cell types or a species of nucleic acid (for example, genomic DNA or mRNA) thereof: a bronchial cell, a bronchiolar cell, an alveolar cell (for example, an alveolar type I cell, an alveolar type II cell, or an alveolar macrophage), an epithelial basement membrane cell, an endothelial cell, an airway epithelial cell (for example, a goblet cell, a ciliated cell, a clara cell, a neuroendocrine cell, a basal cell, an intermediate cell, a serous cell, a brush cell, an oncocyte, a nonciliated columnar cell, a metaplastic cell, a squamous cell, a clara-mucous cell, or a bronchiolar metaplasia cell), a salivary gland cell (for example, a salivary gland serous cell, mucous cell, or ductal cell), an interstitial connective tissue cell (for example, a smooth muscle cell, a cartilage cell, a fibroblast cell, a myofibroblast cell, a meningothelioid cell, an adipose tissue cell, or a neural cell), a blood vessel cell (for example, an endothelial cell, a smooth muscle cell, a fibroblast cell, a myofibroblast cell, a pericyte cell, a lymphatic cell, a hematopoietic cell, a lymphoid tissue cell (for example, a lymphocyte, a plasma cell, a megakaryocyte cell, a macrophage cell, a langerhans cell, a mast cell, an eosinophil cell, a neutrophil cell, or a basophil cell), a pleural cell (for example, a mesothelial cell, a pleuripotent submesothelial fibroblast cell, or an adipose cell), a stem cell, a perivascular epithelioid cell, a pluripotent epithelial stem cell, a meningothelioid cell, or an endothelial progenitor cell. In some embodiments, the subject sample is or comprises a fluid (or associated genetic material), wherein the fluid is selected from the group consisting of blood, plasma, mucus, urine, and lymphatic fluid. In some embodiments, a subject sample, is or comprises a genome or transcriptome, of any of the foregoing cells or fluids. In some embodiments, a subject sample, is or comprises a genome or transcriptome of a tumor cell, wherein the tumor cell is selected from the group consisting of the foregoing cells and fluids. In some embodiments, a subject sample is or comprises a genome or transcriptome of a tumor, wherein the tumor is comprised of one or more cells selected from the group consisting of the foregoing cells. In some embodiments, a subject sample is or comprises a genome or transcriptome of a tumor, wherein the tumor is comprised of one or more fluids selected from the group consisting of the foregoing fluids.

In some embodiments described herein, the subject is diagnosed with, in need of treatment for, being treated for, in remission of, at risk of developing, or predisposed to developing a disease or disorder, for example, a cancer. In some embodiments, the subject is diagnosed with a disease or disorder selected from the group consisting of non-small cell lung cancer, lung adenocarcinoma, lung squamous cell carcinoma, a bladder cancer (for example, bladder urothelial carcinoma), a cervical cancer (for example, cervical squamous cell carcinoma), a uterine cancer (for example, uterine endometrial carcinoma), an esophageal cancer (for example, esophageal squamous cell carcinoma), a head and neck cancer (for example, head and neck squamous cell carcinoma), a kidney cancer (for example, papillary renal cell carcinoma), a breast cancer, colorectal cancer, a melanoma, a stomach cancer, castration-resistant prostate cancer (CRPC), T-cell acute lymphoblastic leukemia (T-ALL), acute myeloid leukemia (AML), myelodysplastic syndrome (MDS), and a liver cancer (for example, hepatocellular carcinoma). In particular embodiments, the subject is diagnosed with the disease or disorder, for example, a cancer.

In some embodiments of a method described herein, the subject tumor genome comprises one or more mutations associated with a disease or disorder, for example a cancer. In some embodiments, the subject tumor genome comprises a somatic mutation in one or more of the NFE2L2, KEAP1, or CUL3 gene sequences. For example, in some embodiments, the subject tumor genome comprises a somatic mutation: in NFE2L2; in KEAP1; in CUL3; in each of NFE2L2 and KEAP1; in each of NFE2L2 and CUL3; in each of KEAP1 and CUL3; or in each of NFE2L2, KEAP1, and CUL3.

In some embodiments of a method described herein, a somatic mutation present in or detected in a subject sample is absent from a control sample or a control data set. For example, in some instances a subject tumor genome comprises or is characterized by a somatic mutation in one or more of the NFE2L2, KEAP1, or CUL3 gene sequences, and the somatic mutation is absent from a control sample or a control data set or the control sample or control data set is characterized by the absence of the somatic mutation. In some instances a somatic mutation in one or more of a NFE2L2, KEAP1, or CUL3 gene sequence is detected in a subject tumor genome and the somatic mutation is not detected in a control sample or a control data set.

In some embodiments, a subject sample characterized by the presence of a somatic mutation or group of somatic mutations can be compared to a control sample or a control data set characterized by the absence of the somatic mutation or the group of somatic mutations. Thus, in some embodiments, the somatic mutation or group of somatic mutations detected in or present in a subject sample is absent from a control sample or a control data set. In some embodiments, the somatic mutation or group of somatic mutations is detected in or present in a subject sample at a higher frequency relative to a control sample or a control data set. Detecting, detection of, or the presence of a somatic mutation or group of somatic mutations in a subject sample can indicate that the subject is in need of treatment with a compound of formula (I), or a pharmaceutically acceptable salt thereof. For example, in some embodiments, detecting, detection of, or the presence of a somatic mutation or group of somatic mutations in a subject sample and absence of the somatic mutation or group of somatic mutations from a control sample or a control data set can indicate that the subject is in need of treatment with a compound of formula (I), or a pharmaceutically acceptable salt thereof. In some embodiments, detecting, detection of, or the presence of a somatic mutation or group of somatic mutations in a subject sample at a higher frequency relative to a control sample or a control data set can indicate that the subject is in need of treatment with a compound of formula (I), or a pharmaceutically acceptable salt thereof.

In various embodiments described herein, a control sample is or comprises a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, or a non-cancerous fluid of a control subject. In some embodiments, a control sample comprises a nucleic acid species, for example, genomic DNA or mRNA, of: a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, or a non-cancerous fluid of a control subject. For example, in some embodiments, a control sample comprises genomic sequence data from a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, or a combination thereof. In some embodiments a control sample comprises a non-cancerous cell genome or transcriptome, for example, a genome or transcriptome of a non-cancerous cell of the subject, or a genome or transcriptome of a non-cancerous cell of a control subject.

In some embodiments, a control data set comprises nucleic acid (for example, genomic DNA or mRNA) data from a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, or a combination thereof. For example, in some embodiments, a control data set comprises genomic sequence data from a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, or a combination thereof. In some embodiments a control data set comprises genomic or transcriptomic data from one or more non-cancerous cell genomes or transcriptomes, for example, genomes or transcriptomes of one or more non-cancerous cells of the subject, or genomes or transcriptomes of one or more non-cancerous cells of a control subject.

In some embodiments, the control sample is or comprises a cell (or genomic DNA or mRNA thereof), wherein the cell is a lung cell, a bladder cell, a stomach cell, a prostate cell, an esophageal cell, a gastrointestinal cell, a lymphatic cell, a nervous system cell, an ovary cell, a cervical cell, a vaginal cell, a pancreatic cell, a throat cell, an esophageal cell, a kidney cell, a small intestine cell, a large intestine cell, a blood cell, a red blood cell, a white blood cell, a platelet cell, or a liver cell. In some embodiments, the control sample is or comprises a cell (or genomic DNA or mRNA thereof), wherein the cell is a lung cell. In some embodiments, the control sample is or comprises a cell (or genomic DNA or mRNA thereof), wherein the cell is a cell of the respiratory tract. For example, in some embodiments, the cell is or comprises a bronchial cell, a bronchiolar cell, an alveolar cell (for example, an alveolar type I cell, an alveolar type II cell, or an alveolar macrophage), an epithelial basement membrane cell, an endothelial cell, an airway epithelial cell (for example, a goblet cell, a ciliated cell, a clara cell, a neuroendocrine cell, a basal cell, an intermediate cell, a serous cell, a brush cell, an oncocyte, a nonciliated columnar cell, a metaplastic cell, a squamous cell, a clara-mucous cell, or a bronchiolar metaplasia cell), a salivary gland cell (for example, a salivary gland serous cell, mucous cell, or ductal cell), an interstitial connective tissue cell (for example, a smooth muscle cell, a cartilage cell, a fibroblast cell, a myofibroblast cell, a meningothelioid cell, an adipose tissue cell, or a neural cell), a blood vessel cell (for example, an endothelial cell, a smooth muscle cell, a fibroblast cell, a myofibroblast cell, a pericyte cell, a lymphatic cell, a hematopoietic cell, a lymphoid tissue cell (for example, a lymphocyte, a plasma cell, a megakaryocyte cell, a macrophage cell, a langerhans cell, a mast cell, an eosinophil cell, a neutrophil cell, or a basophil cell), a pleural cell (for example, a mesothelial cell, a pleuripotent submesothelial fibroblast cell, or an adipose cell), a stem cell, a perivascular epithelioid cell, a pluripotent epithelial stem cell, a meningothelioid cell, or an endothelial progenitor cell. In some embodiments, the control sample is or comprises a fluid, wherein the fluid is selected from the group consisting of blood, plasma, mucus, urine, and lymphatic fluid. In some embodiments a control sample comprises a non-cancerous cell genome or transcriptome (for example, a genome or transcriptome of a non-cancerous cell of a subject or a genome or transcriptome of a non-cancerous cell of a control subject) of any of the foregoing cells or fluids.

In some embodiments, a control data set comprises genomic DNA or mRNA data from a cell, wherein the cell is a lung cell, a bladder cell, a stomach cell, a prostate cell, an esophageal cell, a gastrointestinal cell, a lymphatic cell, a nervous system cell, an ovary cell, a cervical cell, a vaginal cell, a pancreatic cell, a throat cell, an esophageal cell, a kidney cell, a small intestine cell, a large intestine cell, a blood cell, a red blood cell, a white blood cell, a platelet cell, or a liver cell. In some embodiments, the control data set comprises genomic DNA or mRNA data from a cell, wherein the cell is a lung cell. In some embodiments, the control data set comprises genomic DNA or mRNA data from a cell, wherein the cell is a cell of the respiratory tract. For example, in some embodiments, the cell is or comprises a bronchial cell, a bronchiolar cell, an alveolar cell (for example, an alveolar type I cell, an alveolar type II cell, or an alveolar macrophage), an epithelial basement membrane cell, an endothelial cell, an airway epithelial cell (for example, a goblet cell, a ciliated cell, a clara cell, a neuroendocrine cell, a basal cell, an intermediate cell, a serous cell, a brush cell, an oncocyte, a nonciliated columnar cell, a metaplastic cell, a squamous cell, a clara-mucous cell, or a bronchiolar metaplasia cell), a salivary gland cell (for example, a salivary gland serous cell, mucous cell, or ductal cell), an interstitial connective tissue cell (for example, a smooth muscle cell, a cartilage cell, a fibroblast cell, a myofibroblast cell, a meningothelioid cell, an adipose tissue cell, or a neural cell), a blood vessel cell (for example, an endothelial cell, a smooth muscle cell, a fibroblast cell, a myofibroblast cell, a pericyte cell, a lymphatic cell, a hematopoietic cell, a lymphoid tissue cell (for example, a lymphocyte, a plasma cell, a megakaryocyte cell, a macrophage cell, a langerhans cell, a mast cell, an eosinophil cell, a neutrophil cell, or a basophil cell), a pleural cell (for example, a mesothelial cell, a pleuripotent submesothelial fibroblast cell, or an adipose cell), a stem cell, a perivascular epithelioid cell, a pluripotent epithelial stem cell, a meningothelioid cell, or an endothelial progenitor cell. In some embodiments, the control data set comprises biomarker level data from a fluid, wherein the fluid is selected from the group consisting of blood, plasma, mucus, urine, and lymphatic fluid. In some embodiments a control data set comprises genomic or transcriptomic data from one or more non-cancerous cell genomes or transcriptomes (for example, genomes or transcriptomes of one or more non-cancerous cells of the subject, or genomes or transcriptomes of one or more non-cancerous cells of a control subject), wherein the genomic or transcriptomic data is from one or more of the foregoing cell types and fluids.

In some embodiments, the subject sample and the control sample comprise the same type of cell, cell population, cell lysate, tissue, or fluid (or the protein, mRNA, or genomic DNA content thereof). In some embodiments, the subject sample genomic DNA or mRNA is characterized in a cell, a cell population, a cell lysate, a tissue, or a fluid, and the genomic DNA or mRNA of the control data set is characterized in the same or a comparable type of cell, cell population, cell lysate, tissue, or fluid. For example, in some embodiments, the subject sample comprises a lung cell and the control sample comprises a lung cell. For example, in some embodiments, the subject sample comprises a lung cell and the control data set comprises genomic DNA or mRNA data from a lung cell. For example, in some embodiments, the subject sample comprises the genome or transcriptome of a lung cell and the control sample comprises the genome or transcriptome of a lung cell. For example, in some embodiments, the subject sample comprises the genome or transcriptome of a lung cell and the control data set comprises genomic or transcriptomic data from the genome or transcriptome of a lung cell.

Detecting a somatic mutation, for example, detecting a somatic mutation in a subject sample, can be performed using techniques known to those of skill in the art. For example, in some embodiments, detecting a somatic mutation (for example, detecting a somatic mutation in at least one of NFE2L2, KEAP1, or CUL3) comprises sequencing genomic DNA of a subject sample, for example, by PCR, for example, by quantitative PCR (qPCR) or digital PCR. In some embodiments, detecting a somatic mutation (for example, detecting a somatic mutation in at least one of NFE2L2, KEAP1, or CUL3) comprises sequencing mRNA of a subject sample, for example, by RNA-Seq (for example, mRNA-Seq), a reverse transcription polymerase chain reaction (RT-PCR), a reverse transcription quantitative PCR (RT-qPCR), or digital PCR. In some embodiments, genomic DNA of the NFE2L2, KEAP1, and/or CUL3 gene sequence of a subject sample is sequenced. In some embodiments, NFE2L2, KEAP1, and/or CUL3 mRNA (for example, mRNA transcribed from the NFE2L2, KEAP1, and/or CUL3 gene sequence) of a subject sample is sequenced.

In some embodiments of a method described herein, detecting a somatic mutation comprises sequencing genomic DNA of a subject sample and/or a control sample. In some embodiments, the method of genomic DNA sequencing is a high-throughput or next-generation method of sequencing. In some embodiments, the sequencing or method of sequencing genomic DNA is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, and Heliscope single molecule sequencing. In some embodiments, the method of genomic DNA sequencing is a single molecule method of sequencing. In some embodiments, the sequencing or method of sequencing genomic DNA is or comprises performing a PCR, a quantitative PCR (qPCR), or Sanger sequencing. Thus, in some embodiments, detecting a somatic mutation comprises sequencing genomic DNA of a subject sample and the sequencing or method of sequencing is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, digital PCR, Heliscope single molecule sequencing, PCR, a quantitative PCR (qPCR), and Sanger sequencing. In some embodiments, detecting a somatic mutation comprises performing in situ hybridization (ISH) (for example, fluorescence in situ hybridization (FISH), multicolor FISH, in situ PCR, and Chromogenic In Situ Hybridization (CISH)).

Detecting a somatic mutation (for example, a somatic mutation of an NFE2L2, KEAP1, or CUL3 genomic DNA or mRNA sequence) in a subject sample can be indicative that a subject is in need of treatment with a compound of formula (I), or a pharmaceutically acceptable salt thereof. Thus, in some embodiments, if sequencing genomic DNA or RNA (for example mRNA) of a subject sample detects or results in detecting a somatic mutation (for example, a somatic mutation of an NFE2L2, KEAP1, or CUL3 gene sequence) in the subject sample, the subject is in need of treatment with a compound of formula (I), or a pharmaceutically acceptable salt thereof.

In some embodiments, a method described herein includes sequencing a control sample, for example, sequencing genomic DNA or mRNA of a control sample (for example, sequencing genomic DNA or mRNA of at least one of NFE2L2, KEAP1, or CUL3, of a control sample). In some embodiments, the method comprises sequencing genomic DNA of the NFE2L2, KEAP1, and/or CUL3 gene sequence of a control sample. In some embodiments, the method comprises sequencing NFE2L2, KEAP1, and/or CUL3 mRNA of a control sample. In some embodiments, a method described herein comprises sequencing mRNA of a control sample, for example, by RNA-Seq (for example, mRNA-Seq), a reverse transcription polymerase chain reaction (RT-PCR), a reverse transcription quantitative PCR (RT-qPCR), or digital PCR. In some embodiments, a method described herein comprises sequencing genomic DNA of a control sample, and the sequencing or method of sequencing is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, Heliscope single molecule sequencing, PCR, a quantitative PCR (qPCR), digital PCR, and Sanger sequencing.

A method described herein can include the step of comparing sequencing data (for example, mRNA or genomic sequencing data) from a subject sample with a control sample or a control data set. The presence or detection of a somatic mutation in a subject sample and the absence or absence of detection of the somatic mutation in a control sample or a control data set can be indicative that a subject is in need of treatment with a compound of formula (I), or a pharmaceutically acceptable salt thereof. Thus, in some embodiments, a method described herein includes comparing sequencing data (for example, sequencing data of genomic DNA sequencing, or sequencing data of mRNA sequencing) of the subject sample with sequencing data of a control sample or a control data set. Thus, in some embodiments, if sequencing genomic DNA or RNA (for example, mRNA) of a subject sample detects or results in detecting a somatic mutation (for example, a somatic mutation of an NFE2L2, KEAP1, or CUL3 gene sequence) in the subject sample, and sequencing genomic DNA or RNA (for example, mRNA) of a control sample does not detect or does not result in detecting the somatic mutation in the control sample, the subject is in need of treatment with a compound of formula (I), or a pharmaceutically acceptable salt thereof.

In some embodiments, detecting a somatic mutation in a subject sample at a higher frequency relative to detecting the somatic mutation in a control sample or a control data set can be indicative that a subject is in need of treatment with a compound of formula (I), or a pharmaceutically acceptable salt thereof. Thus, in some embodiments, if sequencing genomic DNA or RNA (for example, mRNA) of a subject sample and a control sample detects or results in detecting a somatic mutation (for example, a somatic mutation of an NFE2L2, KEAP1, or CUL3 gene sequence) at a higher frequency in the subject sample relative to a control sample, the subject is in need of treatment with a compound of formula (I), or a pharmaceutically acceptable salt thereof. In some embodiments, if sequencing genomic DNA or RNA (for example, mRNA) of a subject sample detects or results in detecting a somatic mutation (for example, a somatic mutation of an NFE2L2, KEAP1, or CUL3 gene sequence) at a higher frequency in the subject sample relative to the frequency of or frequency of detecting the somatic mutation in a control data set, the subject is in need of treatment with a compound of formula (I), or a pharmaceutically acceptable salt thereof.

In some embodiments, a subject sample is characterized by the presence of a somatic mutation. In some embodiments, the subject sample is characterized by the presence of the somatic mutation by sequencing genomic DNA of the subject sample. A sample, for example, a subject sample or a control sample, can be characterized by the presence of a somatic mutation using techniques known to those of skill in the art. For example, in some embodiments, the subject sample is characterized by the presence of the somatic mutation (for example, a somatic mutation in at least one of a NFE2L2, KEAP1, or CUL3 gene sequence) by sequencing genomic DNA of a subject sample. In some embodiments, the subject sample is characterized by the presence of the somatic mutation (for example, a somatic mutation in at least one of a NFE2L2, KEAP1, or CUL3 gene sequence) by sequencing mRNA of a subject sample, for example, by RNA-Seq (for example, mRNA-Seq), a reverse transcription polymerase chain reaction (RT-PCR), or a reverse transcription quantitative PCR (RT-qPCR). In some embodiments, genomic DNA of the NFE2L2, KEAP1, and/or CUL3 gene sequence of a subject sample is sequenced. In some embodiments, NFE2L2, KEAP1, and/or CUL3 mRNA of a subject sample is sequenced.

In some embodiments of a method described herein, a subject sample is characterized by the presence of a somatic mutation (for example, a somatic mutation in at least one of an NFE2L2, KEAP1, or CUL3 gene sequence) by sequencing genomic DNA of a subject sample. In some embodiments of a method described herein, a control sample is characterized by the absence of a somatic mutation (for example, a somatic mutation in at least one of a NFE2L2, KEAP1, or CUL3 gene sequence) by sequencing genomic DNA of the control sample. In some embodiments, the method of genomic DNA sequencing is a high-throughput or next-generation method of sequencing. In some embodiments, the method of sequencing is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, and Heliscope single molecule sequencing. In some embodiments, the method of genomic DNA sequencing is a single molecule method of sequencing. In some embodiments, the sequencing or method of sequencing genomic DNA is or comprises performing a PCR, a quantitative PCR (qPCR), digital PCR, or Sanger sequencing.

Thus, in some embodiments, a subject sample is characterized by the presence of a somatic mutation (for example, a somatic mutation in at least one of a NFE2L2, KEAP1, or CUL3 gene sequence) by sequencing genomic DNA of a subject sample and the sequencing or method of sequencing is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, Heliscope single molecule sequencing, PCR, a quantitative PCR (qPCR), digital PCR, and Sanger sequencing. In some embodiments, a subject sample is characterized by the presence of a somatic mutation (for example, a somatic mutation in at least one of NFE2L2, KEAP1, or CUL3 gene sequence) by sequencing genomic DNA of a subject sample and the sequencing or method of sequencing is PCR.

A subject sample characterized by the presence of a somatic mutation (for example, a somatic mutation present in an NFE2L2, KEAP1, or CUL3 gene sequence) can be indicative that a subject is in need of treatment with a compound of formula (I), or a pharmaceutically acceptable salt thereof. Thus, in some embodiments, if a subject sample is characterized by the presence of a somatic mutation (for example, if sequencing genomic DNA or RNA (for example, mRNA) of a subject sample characterizes the subject sample by the presence of a somatic mutation (for example, a somatic mutation present in an NFE2L2, KEAP1, or CUL3 gene sequence)), the subject is in need of treatment with a compound of formula (I), or a pharmaceutically acceptable salt thereof.

In some embodiments of a method described herein, a control sample is characterized by the absence of a somatic mutation (for example, a somatic mutation in at least one of NFE2L2, KEAP1, or CUL3) by sequencing genomic DNA or RNA (for example, mRNA) of the control sample. In some embodiments, the method of sequencing genomic DNA is a high-throughput or next-generation method of sequencing. In some embodiments, a control sample is characterized by the absence of a somatic mutation by sequencing mRNA of a control sample, for example, by RNA-Seq (for example, mRNA-Seq), a reverse transcription polymerase chain reaction (RT-PCR), digital PCR, or a reverse transcription quantitative PCR (RT-qPCR). In some embodiments, a control sample is characterized by the absence of a somatic mutation by sequencing genomic DNA of a control sample, and the sequencing or method of sequencing is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, Heliscope single molecule sequencing, PCR, a quantitative PCR (qPCR), digital PCR, and Sanger sequencing. In some embodiments, the sequencing or method of sequencing is PCR.

In some embodiments, a subject sample is characterized by the presence of a somatic mutation and/or a control sample or control data set is characterized by the absence of the somatic mutation, indicating that a subject is in need of treatment with a compound of formula (I), or a pharmaceutically acceptable salt thereof. Thus, in some embodiments, if sequencing (for example, sequencing genomic DNA or RNA (for example mRNA)) of a subject sample detects or results in detecting a somatic mutation (for example, a somatic mutation present in an NFE2L2, KEAP1, or CUL3 gene sequence), and sequencing (for example, sequencing genomic DNA or RNA (for example, mRNA)) of a control sample does not detect or does not result in detecting the somatic mutation, the subject is in need of treatment with a compound of formula (I), or a pharmaceutically acceptable salt thereof.

In some embodiments, a subject sample is characterized by the presence of a somatic mutation at a higher frequency relative to a control sample or a control data set, indicating that a subject is in need of treatment with a compound of formula (I), or a pharmaceutically acceptable salt thereof. Thus, in some embodiments, if a subject sample is characterized by the presence of a somatic mutation at a higher frequency relative to a control sample or a control data set, the subject is in need of treatment with a compound of formula (I), or a pharmaceutically acceptable salt thereof.

The invention described herein includes, in another aspect, the use of an AKR1C3 level for selecting a subject for treatment with a compound of formula (I), or a pharmaceutically acceptable salt thereof. Thus, also described herein is the use of an AKR1C3level for selecting a subject for treatment with a compound of formula (I), or a pharmaceutically acceptable salt thereof, wherein a subject is treated with the compound of formula (I), or a pharmaceutically acceptable salt thereof, if a sample of the subject is characterized as having an elevated AKR1C3 level.

The invention described herein includes, in yet another aspect, the use of a somatic mutation for selecting a subject for treatment with a compound of formula (I), or a pharmaceutically acceptable salt thereof. Thus, also described herein is the use of a somatic mutation for selecting a subject for treatment with a compound of formula (I), or a pharmaceutically acceptable salt thereof, wherein a subject is treated with the compound of formula (I), or a pharmaceutically acceptable salt thereof, if a sample of the subject is characterized by the presence of the somatic mutation and wherein the somatic mutation is detected in one of the following genes: NFE2L2, KEAP1, or CUL3.

In some embodiments of a use described herein, the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof. In some embodiments of a use described herein, the compound of formula (I) is 6′-fluoro-N-(4-fluorobenzyl)-4′-oxo-3′,4′-dihydro-1′H-spiro[piperidine-4,2′-quinoline]-1-carboxamide, or a pharmaceutically acceptable salt thereof. In some embodiments of a use described herein, the compound of formula (I) is N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof.

Also described herein is a kit suitable for performing a method described herein or for a use described herein. For example, described herein are kits for: identifying a subject for treatment with a compound of formula (I), or a pharmaceutically acceptable salt thereof, selecting a compound of formula (I), or a pharmaceutically acceptable salt thereof, for treating a subject; determining in a subject sample a level of AKR1C3; or characterizing a subject sample as having an elevated level of AKR1C3. Also described herein are kits for: detecting in a subject sample a somatic mutation in at least one of the following genes: NFE2L2, KEAP1, or CUL3; or characterizing a subject sample for the presence of a somatic mutation in at least one of the following genes: NFE2L2, KEAP1, or CUL3. A kit described herein can include components suitable for determining in a sample (for example, a subject sample or a control sample) a level of AKR1C3. A kit described herein can include components suitable for detecting a somatic mutation in one or more gene sequences, for example, an NFE2L2, KEAP1, or CUL3 gene sequence. A kit described herein can include components suitable for detecting a somatic mutation in an NFE2L2, KEAP1, or CUL3 gene sequence, wherein the method comprises sequencing RNA (for example, mRNA) transcribed from a NFE2L2, KEAP1, or CUL3 gene sequence. In some embodiments, a kit described herein comprises components suitable for determining an mRNA or protein level of a biomarker. In some embodiments, a kit described herein comprises components suitable for performing an antigen detection assay, for example, an IHC assay. In some embodiments, a kit described herein comprises components suitable for sequencing genomic DNA or RNA (for example, mRNA).

Also described herein is an assay suitable for: identifying a subject for treatment with a compound of formula (I), or a pharmaceutically acceptable salt thereof; selecting a compound of formula (I), or a pharmaceutically acceptable salt thereof, for treating a subject; determining in a subject sample a level of AKR1C3; or characterizing a subject sample as having an elevated level of AKR1C3. Also described herein are assays for: detecting in a subject sample a somatic mutation in at least one of the following genes: NFE2L2, KEAP1, or CUL3; or characterizing a subject sample for the presence of a somatic mutation in at least one of the following genes: NFE2L2, KEAP1, or CUL3. An assay described herein can include steps suitable for determining in a sample (for example, a subject sample or a control sample) a level of AKR1C3. An assay described herein can include steps suitable for detecting a somatic mutation in one or more gene sequences, for example, an NFE2L2, KEAP1, or CUL3 gene sequence. An assay described herein can include steps suitable for detecting a somatic mutation in an NFE2L2, KEAP1, or CUL3 gene sequence by sequencing RNA (for example, mRNA) transcribed from an NFE2L2, KEAP1, or CUL3 gene sequence. In some embodiments, an assay described herein includes steps suitable for determining an mRNA or protein level of AKR1C3. In some embodiments, an assay described herein includes steps suitable for performing an antigen detection assay, for example, an IHC assay. In some embodiments, an assay described herein includes steps suitable for sequencing genomic DNA or RNA (for example, mRNA).

A pharmaceutical composition comprising a compound of formula (I), or a pharmaceutically acceptable salt thereof, can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound of formula (I) or a pharmaceutical composition thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable using in vitro and in vivo tests using advantageously mammals, e.g., mice, rats, dogs, monkeys or isolated organs, tissues and preparations thereof. The compounds of formula (I) can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

### BRIEF DESCRIPTION OF THE DRAWINGS

FIG. 1 is a graph showing H-scores calculated for samples of various tumor types including: non-small cell lung cancer (“NSCLC”)(Adenocarcinoma and Squamous cell carcinoma subtypes), prostate cancer (“Prostate”), and hepatocellular carcinoma (“HCC”). Also shown are H-scores for clinical trial biopsies from NSCLC, HCC, and head and neck cancer (“H&N”) patient tumors harboring NFE2L2/KEAP1 mutations. Shown are the percentage of samples and clinical trial biopsies that were assigned an H-score of greater than 250 out of 300.

FIG. 2 is a schematic of the clinical trial study design for a clinical trial to determine AKR1C3-dependent KARS inhibitor dosing.

FIG. 3 is a schematic showing design of safety and efficacy assessments during screening/baseline and during treatment for a clinical trial to determine AKR1C3-dependent KARS inhibitor dosing.

### DETAILED DESCRIPTION OF THE INVENTION

Various embodiments of the invention are described herein. It will be recognized that features specified in each embodiment may be combined with other specified features to provide further embodiments of the present invention.

The present disclosure is based at least in part on the identification of a compound that inhibits AKR1C3 and methods of use of the same compound to treat AKR1C3-associated diseases. Disclosed herein is Compound (I) and Compound (II), and pharmaceutical compositions thereof:

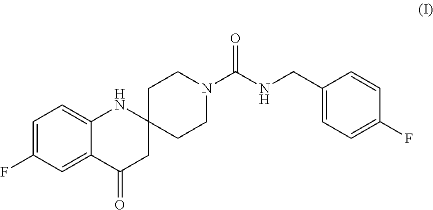 [SMILES: O=C1CC2(CCN(C(=O)NCc3ccc(F)cc3)CC2)Nc2ccc(F)cc21]

Compound of Formula (I), 6′-fluoro-N-(4-fluorobenzyl)-4′-oxo-3′,4′-dihydro-1′H-spiro[piperidine-4,2′-quinoline]-1-carboxamide;

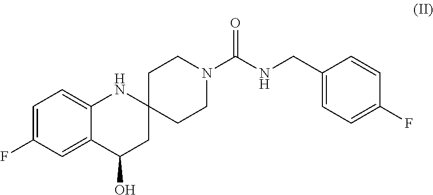 [SMILES: O=C(NCc1ccc(F)cc1)N1CCC2(CC1)C[C@@H](O)c1cc(F)ccc1N2]

Compound of Formula (II), (R)-6′-fluoro-N-(4-fluorobenzyl)-4′-hydroxy-3′,4′-dihydro-1′H-spiro[piperidine-4,2′-quinoline]-1-carboxamide, are activated in a variety of assays and therapeutic models, acting as a selective AKR1C3 inhibitor.

In one aspect, the invention provides a method of identifying a subject for treatment with a compound of formula (I):

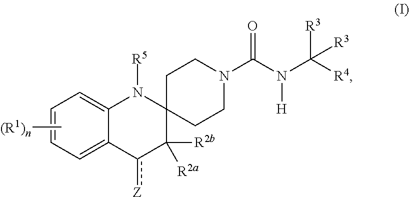

wherein [symbol] is a single bond or a double bond;
Z is either OH, when [symbol] is a single bond; or O, when [symbol] is a double bond;
each R¹ is independently selected from the group consisting of, (C₁-C₆)alkyl, (C₁-C₆)alkoxy, (C₀-C₄)alkylN(R⁸)₂, and halo;
R^{2a} and R^{2b} are each independently selected from the group consisting of H, (C₁-C₆) alkyl, and halo;
each R³ is independently selected from the group consisting of H, and halo;
R⁴ is selected from the group consisting of aryl, a 5 to 6-membered heteroaryl comprising 1, 2, 3, or 4 heteroatoms independently selected from N, O, and S; and a 9 to 10-membered fused bicyclic heteroaryl comprising 1, 2, 3, or 4 heteroatoms independently selected from N, O, and S; wherein any of the foregoing is optionally substituted with one or more R⁶;
R⁵ is selected from the group consisting of H; (C₁-C₆)alkyl; (C₂-C₆)alkenyl; (C₀-C₄)alkylOR⁸; (C₁-C₄)alkyl(C₃-C₁₀)cycloalkyl; halo(C₁-C₆)alkyl; (C₂-C₃)alkynyl; (C₁-C₄)alkylN(R¹⁰)₂;
each R⁶ is independently selected from the group consisting of halo; (C₁-C₆)alkyl; (C₁-C₆)alkoxy; halo(C₁-C₆)alkyl; OH; aryl; 3 to 6-membered heterocycle; 5- to 6-membered heteroaryl; (C₀-C₄)alkylS(O)ₘ(C₁-C₆)alkyl; halo(C₁-C₆)alkoxy; (C₀-C₄)alkylS(O)ₘN(R⁸)₂; (C₀-C₄)alkyl N(R⁸)₂; (C₀-C₄)alkyl(CO)OR⁷; N(R⁸)S(O)ₘ(C₁-C₆)alkyl; N(R⁸)S(O)ₘ(C₃-C₆)cycloalkyl; OP(O)(OH)₂; (C₀-C₃)alkyl(CO)NHR¹¹; (C₀-C₃)alkylOR⁷, and (C₃-C₁₀)cycloalkyl; wherein each R⁶, when not being halo, OH, or OP(O)(OH)₂, is optionally substituted with one to three R⁹; or two neighboring R⁶, together with the atoms to which they attach form a 5 to 7-membered heterocycle or (C₅-C₈)cycloalkyl;
each R⁷ and R⁸ is independently selected from the group consisting of H or (C₁-C₆)alkyl, that is optionally substituted with one to three R⁹;
each R⁹ is independently selected from the group consisting of halo; —OH; amino, (C₁-C₄)alkylamino, di(C₁-C₄)alkylamino, OP(O)(OH)₂; (C₁-C₆)alkyl; (C₁-C₃)alkynyl; (C₁-C₆)alkoxy; halo(C₁-C₆)alkyl; (C₀-C₄)alkylS(O)ₘ(C₁-C₆)alkyl; halo(C₁-C₆)alkoxy; 3 to 6-membered heterocycle which is optionally substituted with oxo (═O); (C₀-C₄)alkylS(O)ₘN(R¹⁰)₂; (C₀-C₄)alkyl(CO)R¹⁰; (C₀-C₄)alkyl(CO)OR¹⁰; (C₀-C₄)alkylNR¹⁰S(O)ₘ(C₁-C₆)alkyl; (C₀-C₄)alkylOR¹⁰; (C₀-C₄)alkylN(R¹⁰)₂; (C₀-C₄)alkylCN; (C₀-C₄)alkylN(R¹⁰)₂; and (C₀-C₄)alkyl(CO)N(R¹⁰)₂;
each R¹⁰ is independently selected from the group consisting of H, (C₁-C₆)alkyl; or 3 to 6-membered heterocycle, wherein the 3 to 6-membered heterocycle is optionally substituted with one or more of (C₁-C₆)alkyl; and oxo (═O);
each R¹¹ is selected from the group consisting of H; 4 to 6-membered heterocycle which is optionally substituted with one to four R¹²; (C₃-C₆)cycloalkyl which is optionally substituted with one to four R¹²; (C₀-C₃)alkyl(C₃-C₆)cycloalkyl (C₁-C₃)alkyl which is optionally substituted with halo; CH₂-aryl which is optionally substituted with one to three R¹²; (C₁-C₆)alkyl; (C₂-C₆)alkenyl; or (C₂-C₆)alkynyl, wherein each of the (C₁-C₆)alkyl; (C₂-C₆)alkenyl; and (C₂-C₆)alkynyl is optionally substituted with one or more R¹³;
each R¹² is independently selected from the group consisting of OH, (C₁-C₃)alkoxy, NH₂; or (C₁-C₃)alkyl optionally substituted with one or more OH;
each R¹³ is independently selected from the group consisting of halo, OH, amino, (C₁-C₄)alkylamino, di(C₁-C₄)alkylamino, (C₁-C₃)alkoxy; and C(O)—(C₃-C₅)cycloalkyl;
m is 0, 1, or 2; and
n is 0, 1 or 2,
or a pharmaceutically acceptable salt thereof,
   the method comprising determining in a subject sample a level of AKR1C3, wherein an elevated level of AKR1C3 identifies the subject as a subject in need of treatment with the compound of formula (I), or a pharmaceutically acceptable salt thereof.

Also described herein is a method of selecting a compound of formula (I), or a pharmaceutically acceptable salt thereof, for treating a subject, the method comprising determining in a subject sample a level of AKR1C3, wherein an elevated level of AKR1C3 identifies the subject as a subject in need of treatment of the compound of formula (I), or a pharmaceutically acceptable salt thereof.

In another aspect, the invention provides a method of treating a subject, the method comprising: determining in a subject sample a level of AKR1C3, wherein an elevated level of AKR1C3 identifies the subject as in need of treatment with a compound of formula (I), or a pharmaceutically acceptable salt thereof; and administering an effective amount of the compound of formula (I), or a pharmaceutically acceptable salt thereof, to the subject.

In another aspect, the invention provides a method of treating a subject, the method comprising administering to the subject an effective amount of a compound of formula (I), or a pharmaceutically acceptable salt thereof, wherein prior to said administering, a subject sample is characterized as having an elevated AKR1C3 level.

In another aspect, the invention provides a method of treating a subject with a compound of formula (I), or a pharmaceutically acceptable salt thereof, wherein a subject sample is characterized as having an elevated level of AKR1C3.

In some embodiments, the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof. In some embodiments, the compound of formula (I) is 6′-fluoro-N-(4-fluorobenzyl)-4′-oxo-3′,4′-dihydro-1′H-spiro[piperidine-4,2′-quinoline]-1-carboxamide, or a pharmaceutically acceptable salt thereof.

In some embodiments of a method described herein, the level of AKR1C3 is a level of AKR1C3 protein. In some embodiments of a method described herein, the level of AKR1C3 is a level of an AKR1C3 nucleic acid species, for example, AKR1C3 mRNA.

In some embodiments, the subject sample comprises a cell, a cell population, a cell lysate, a tissue, or a fluid of the subject. In some embodiments of a method described herein, the cell is a cancerous cell. In some embodiments, the cancerous cell is a tumor cell. In some embodiments, the tumor cell is selected from the group consisting of a lung cancer tumor cell, a non-small cell lung cancer tumor cell, a lung adenocarcinoma tumor cell, a lung squamous cell carcinoma cell, a bladder tumor cell, a cervical tumor cell, an esophageal tumor cell, a head and neck tumor cell, a kidney tumor cell, and a liver tumor cell. In some embodiments, the cell is a lung cell. In some embodiments, the fluid is selected from the group consisting of blood, plasma, and lymphatic fluid. In some embodiments, the subject sample comprises a genome, a transcriptome, or a proteome of a cell, for example, a subject tumor cell. In some embodiments, the subject sample comprises a genome, a transcriptome, or a proteome of any of the foregoing cells or fluids.

In some embodiments of a method described herein, the subject is diagnosed with a disease or disorder selected from the group consisting of a non-small cell lung cancer, a lung adenocarcinoma, a lung squamous cell carcinoma, a bladder cancer, a cervical cancer, an esophageal cancer, a head and neck cancer, a kidney cancer, and a liver cancer.

In some embodiments of a method described herein, the subject tumor genome comprises a somatic mutation in one or more of the NFE2L2, KEAP1, or CUL3 gene sequences.

In some embodiments of a method described herein, the AKR1C3 level is elevated relative to a control level of AKR1C3. In some embodiments, the control level comprises a AKR1C3 level of a control sample or a control data set. In some embodiments, the control sample comprises a sample selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, and a non-cancerous fluid of a control subject. In some embodiments, the control data set comprises AKR1C3 level data from a source selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, and a combination thereof. In some embodiments, the control sample comprises a genome, a transcriptome, or a proteome of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, or a non-cancerous fluid of a control subject. In some embodiments, the control sample comprises a genome, a transcriptome, or a proteome of any of the foregoing cells or fluids. In some embodiments, the control data set comprises AKR1C3 level data from a source selected from the group consisting of a genome, a transcriptome, or a proteome of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, and a combination thereof.

In some embodiments of a method described herein, the level of AKR1C3 is a AKR1C3 protein level.

In some embodiments of a method described herein, the level of AKR1C3 is an AKR1C3 RNA level. In some embodiments, the AKR1C3 RNA level is an AKR1C3 mRNA level.

In some embodiments of a method described herein, if the level of AKR1C3 in the subject sample is about 1.5 times greater, about 2 times greater, about 3 times greater, about 4 times greater, about 5 times greater, about 6 times greater, about 7 times greater, about 8 times greater, about 9 times greater, about 10 times greater, about 20 times greater, about 30 times greater, about 40 times greater, about 50 times greater, about 60 times greater, about 70 times greater, about 80 times greater, about 90 times greater, about 100 times greater, about 200 times greater, about 300 times greater, about 400 times greater, about 500 times greater, about 600 times greater, about 700 times greater, about 800 times greater, about 900 times greater, about 1000 times greater, about 1500 times greater, or about 2000 times greater than the level of AKR1C3 in the control sample or the control data set, then the subject is in need of treatment with the compound of formula (I), or a pharmaceutically acceptable salt thereof.

In some embodiments of a method described herein, if the level of AKR1C3 in the subject sample is at least about 1.5 times greater, at least about 2 times greater, at least about 3 times greater, at least about 4 times greater, at least about 5 times greater, at least about 6 times greater, at least about 7 times greater, at least about 8 times greater, at least about 9 times greater, at least about 10 times greater, at least about 20 times greater, at least about 30 times greater, at least about 40 times greater, at least about 50 times greater, at least about 60 times greater, at least about 70 times greater, at least about 80 times greater, at least about 90 times greater, at least about 100 times greater, at least about 200 times greater, at least about 300 times greater, at least about 400 times greater, at least about 500 times greater, at least about 600 times greater, at least about 700 times greater, at least about 800 times greater, at least about 900 times greater, at least about 1000 times greater, at least about 1500 times greater, or at least about 2000 times greater than the level of AKR1C3 in the control sample or the control data set, then the subject is in need of treatment with the compound of formula (I), or a pharmaceutically acceptable salt thereof.

In some embodiments of a method described herein, determining in a subject sample a level of AKR1C3 further comprises performing an antigen detection assay. In some embodiments, the antigen detection assay is selected from the group consisting of a western blot assay, an enzyme-linked immunosorbent assay (ELISA), an immunohistochemistry (IHC) assay, an immunocytochemistry assay, a flow cytometry assay, an immunoprecipitation assay, an immuno-electrophoresis assay, and an immuno-electron microscopy assay. In some embodiments, the antigen detection assay is an IHC assay.

In some embodiments, performing the antigen detection assay comprises probing the subject sample with an AKR1C3 antibody. In some embodiments, the AKR1C3 antibody is an anti-AKR1C3 mouse monoclonal antibody, clone NP6.G6.A6. In some embodiments, the AKR1C3 antibody comprises CDR sequences sharing at least 80% identity, at least 85% identity, at least 90% identity, at least 95% identity, at least 96% identity, at least 97% identity, at least 98% identity, or at least 99% identity with the CDR sequences of anti-AKR1C3 mouse monoclonal antibody, clone NP6.G6.A6. In some embodiments, the AKR1C3 antibody is conjugated to horse radish peroxidase (HRP).

In some embodiments, performing the antigen detection assay further comprises probing the subject sample with a secondary antibody. In some embodiments, the secondary antibody is conjugated to HRP.

In some embodiments, the antigen detection assay further comprises applying 3,3′-diaminobenzidine (DAB) to the subject sample.

In some embodiments of a method described herein, determining in a subject sample a level of AKR1C3, further comprises producing an IHC signal intensity score for the subject sample. In some embodiments, if the IHC signal intensity score for the subject sample is 0.5 or greater, 1.0 or greater, 1.5 or greater, 2.0 or greater, 2.5 or greater, 2.6 or greater, 2.7 or greater, 2.8 or greater, or 2.9 or greater, and the IHC signal intensity score ranges from 0-3.0, then the subject is in need of treatment with the compound of formula (I), or a pharmaceutically acceptable salt thereof. In some embodiments, if the IHC signal intensity score for the subject sample is 50 or greater, 100 or greater, 150 or greater, 200 or greater, 250 or greater, 260 or greater, 270 or greater, 280 or greater, or 290 or greater, and the IHC signal intensity score ranges from 0-300, then the subject is in need of treatment with the compound of formula (I), or a pharmaceutically acceptable salt thereof.

In some embodiments of a method described herein, the subject sample is characterized as having an elevated AKR1C3 level by an antigen detection assay. In some embodiments, the antigen detection assay is selected from the group consisting of a western blot assay, an enzyme-linked immunosorbent assay (ELISA), an immunohistochemistry (IHC) assay, an immunocytochemistry assay, a flow cytometry assay, an immunoprecipitation assay, an immuno-electrophoresis assay, and an immuno-electron microscopy assay. In some embodiments, the antigen detection assay is an IHC assay.

In some embodiments, the antigen detection assay comprises probing the subject sample with an AKR1C3 antibody. In some embodiments, the AKR1C3 antibody is an anti-AKR1C3 mouse monoclonal antibody, clone NP6.G6.A6. In some embodiments, the AKR1C3 antibody comprises CDR sequences sharing at least 80% identity, at least 85% identity, at least 90% identity, at least 95% identity, at least 96% identity, at least 97% identity, at least 98% identity, or at least 99% identity with the CDR sequences of anti-AKR1C3 mouse monoclonal antibody, clone NP6.G6.A6. In some embodiments, the AKR1C3 antibody is conjugated to horse radish peroxidase (HRP).

In some embodiments, the antigen detection assay further comprises probing the subject sample with a secondary antibody. In some embodiments, the secondary antibody is conjugated to HRP.

In some embodiments, the antigen detection assay further comprises applying 3,3′-diaminobenzidine (DAB) to the subject sample.

In some embodiments, the antigen detection assay further comprises producing an IHC signal intensity score for the subject sample. In some embodiments, if the IHC signal intensity score for the subject tissue sample is 0.5 or greater, 1.0 or greater, 1.5 or greater, 2.0 or greater, 2.5 or greater, 2.6 or greater, 2.7 or greater, 2.8 or greater, or 2.9 or greater, and the IHC signal intensity score ranges from 0-3.0, then the subject is in need of treatment with the compound of formula (I), or a pharmaceutically acceptable salt thereof. In some embodiments, if the IHC signal intensity score for the subject tissue sample is 50 or greater, 100 or greater, 150 or greater, 200 or greater, 250 or greater, 260 or greater, 270 or greater, 280 or greater, or 290 or greater, and the IHC signal intensity score ranges from 0-300, then the subject is in need of treatment with the compound of formula (I), or a pharmaceutically acceptable salt thereof. In some embodiments, if the IHC signal intensity score for the subject tissue sample is greater than the IHC signal intensity score for a control sample or a control data set, then the subject is in need of treatment with the compound of formula (I), or a pharmaceutically acceptable salt thereof. In some embodiments, if the IHC signal intensity score for the subject tissue sample is at least 5% higher, at least 10% higher, at least 20% higher, at least 30% higher, at least 40% higher, at least 50% higher, at least 60% higher, at least 70% higher, at least 80% higher, at least 90% higher, or at least 100% higher than the IHC signal intensity score for a control sample or a control data set, then the subject is in need of treatment with the compound of formula (I), or a pharmaceutically acceptable salt thereof.

In some embodiments of a method described herein, determining in a subject sample a level of AKR1C3 comprises performing a polymerase chain reaction (PCR) effective to determine the AKR1C3 level in the subject sample. In some embodiments, determining in a subject sample a level of AKR1C3, further comprises performing a PCR effective to determine the level of a control marker in the subject sample. In some embodiments, the control marker is selected from the group consisting of beta actin and glyceraldehyde-3-phosphate dehydrogenase (GAPDH).

In some embodiments, determining in a subject sample a level of AKR1C3 further comprises performing a PCR effective to determine an AKR1C3 level in a control sample. In some embodiments, determining in a subject sample a level of AKR1C3 further comprises performing a PCR effective to determine the level of a control marker in the control sample. In some embodiments, the control marker is beta actin or GAPDH.

As noted above, in some embodiments of a method described herein, determining in a subject sample a level of AKR1C3 comprises performing a polymerase chain reaction (PCR) effective to determine the AKR1C3 level and/or a control marker in a subject sample or a control sample. In some embodiments, the PCR is a quantitative PCR (qPCR). In some embodiments, the PCR is a RT-PCR. In some embodiments, the PCR is a RT-qPCR. In some embodiments, the PCR is a digital PCR.

In some embodiments of a method described herein, the subject sample is characterized as having an elevated AKR1C3 level by a PCR effective to determine the AKR1C3 level in the subject sample. In some embodiments, a level of AKR1C3 is determined in a control sample by a PCR effective to determine the AKR1C3 level in the control sample. In some embodiments, a level of a control marker is determined in the subject sample by a PCR effective to determine the control marker level in the subject sample. In some embodiments, a level of the control marker is determined in the control sample by a PCR effective to determine the control marker level in the control sample. In some embodiments, the control marker is selected from the group consisting of beta actin and glyceraldehyde-3-phosphate dehydrogenase (GAPDH).

As noted above, in some embodiments of a method described herein, a level of AKR1C3 or a control marker is determined in a subject sample or a control sample by performing a polymerase chain reaction (PCR) effective to determine the AKR1C3 level or the control marker level in the subject sample or the control sample. In some embodiments, the PCR is a qPCR. In some embodiments, the PCR is a RT-PCR. In some embodiments, the PCR is a RT-qPCR. In some embodiments, the PCR is a digital PCR.

In another aspect, the invention provides a method of identifying a subject in need of treatment with a compound of formula (I), or a pharmaceutically acceptable salt thereof, the method comprising detecting in a subject sample a somatic mutation in at least one of the following genes: NFE2L2, KEAP1, or CUL3.

In another aspect, the invention provides a method of selecting a compound of formula (I), or a pharmaceutically acceptable salt thereof, for treating a subject, the method comprising detecting in a subject sample a somatic mutation in at least one of the following genes: NFE2L2, KEAP1, or CUL3.

In another aspect, the invention provides a method of treating a subject, the method comprising: detecting in a subject sample a somatic mutation in at least one of the following genes: NFE2L2, KEAP1, or CUL3, wherein said detecting identifies the subject as in need of treatment with a compound of formula (I), or a pharmaceutically acceptable salt thereof; and administering an effective amount of the compound of formula (I), or a pharmaceutically acceptable salt thereof, to the subject.

In another aspect, the invention provides a method of treating a subject, the method comprising administering to the subject an effective amount of a compound of formula (I), or a pharmaceutically acceptable salt thereof, wherein prior to said administering, a subject sample is characterized by the presence of a somatic mutation in at least one of the following genes: NFE2L2, KEAP1, or CUL3.

In another aspect, the invention provides a method of treating a subject with a compound of formula (I), or a pharmaceutically acceptable salt thereof, wherein a subject sample is characterized by the presence of a somatic mutation in at least one of the following genes: NFE2L2, KEAP1, or CUL3.

In some embodiments of a method described herein, the somatic mutation comprises a mutation selected from the group consisting of a nonsense mutation, a missense mutation, a substitution mutation, a frameshift mutation, a point mutation, an insertion mutation, an amplification mutation (for example, a gene amplification), a deletion mutation (for example, a gene deletion), an inversion mutation, and a duplication mutation. In some embodiments, the somatic mutation is a mutation of a tumor cell genome.

In some embodiments of a method described herein, the somatic mutation comprises a single nucleotide polymorphism (SNP).

In some embodiments of a method described herein, the subject sample comprises a cell, a cell population, a cell lysate, a tissue, or a fluid of the subject. In some embodiments, the subject sample comprises genomic DNA of the cell, the cell population, the cell lysate, the tissue, or the fluid of the subject. In some embodiments, the cell is a cancerous cell. In some embodiments, the cancerous cell is a tumor cell. In some embodiments, the tumor cell is selected from the group consisting of a lung cancer tumor cell, a non-small cell lung cancer tumor cell, a lung adenocarcinoma tumor cell, a lung squamous cell carcinoma cell, a bladder tumor cell, a cervical tumor cell, an esophageal tumor cell, a head and neck tumor cell, a kidney tumor cell, and a liver tumor cell. In some embodiments, the cell is a lung cell. In some embodiments, the fluid is selected from the group consisting of blood, plasma, and lymphatic fluid. In some embodiments, the subject sample comprises the genome or the transcriptome of a cell of the subject, for example, a tumor cell of the subject. In some embodiments, the subject sample comprises the genome or the transcriptome of any of the foregoing cells or fluids of a subject.

In some embodiments of a method described herein, the somatic mutation is absent from a control sample or a control data set. In some embodiments, the control sample comprises a sample selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, and a non-cancerous fluid of a control subject. In some embodiments, the control sample comprises genomic DNA of: the non-cancerous cell of the subject, the non-cancerous cell population of the subject, the non-cancerous tissue of the subject, the non-cancerous fluid of the subject, the non-cancerous cell of a control subject, the non-cancerous cell population of a control subject, the non-cancerous tissue of a control subject, or the non-cancerous fluid of a control subject. In some embodiments, the control data set comprises genomic sequence data from a source selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, and a combination thereof. In some embodiments, the control sample comprises a genome or a transcriptome of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, or a non-cancerous fluid of a control subject. In some embodiments, the control sample comprises a genome or a transcriptome of any of the foregoing cells or fluids. In some embodiments, the control data set comprises genomic or transcriptomic data from a source selected from the group consisting of a genome or a transcriptome= of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, and a combination thereof.

In some embodiments of a method described herein, the level of AKR1C3 (for example, AKR1C3 mRNA or AKR1C3 protein) in the subject sample is about 1.5 times greater, about 2 times greater, about 3 times greater, about 4 times greater, about 5 times greater, about 6 times greater, about 7 times greater, about 8 times greater, about 9 times greater, about 10 times greater, about 20 times greater, about 30 times greater, about 40 times greater, about 50 times greater, about 60 times greater, about 70 times greater, about 80 times greater, about 90 times greater, about 100 times greater, about 200 times greater, about 300 times greater, about 400 times greater, about 500 times greater, about 600 times greater, about 700 times greater, about 800 times greater, about 900 times greater, about 1000 times greater, about 1500 times greater, or about 2000 times greater than a level of AKR1C3 (for example, AKR1C3 mRNA or AKR1C3 protein) in a control sample or a control data set.

In some embodiments of a method described herein, the level of AKR1C3 (for example, AKR1C3 mRNA or AKR1C3 protein) in the subject sample is at least about 1.5 times greater, at least about 2 times greater, at least about 3 times greater, at least about 4 times greater, at least about 5 times greater, at least about 6 times greater, at least about 7 times greater, at least about 8 times greater, at least about 9 times greater, at least about 10 times greater, at least about 20 times greater, at least about 30 times greater, at least about 40 times greater, at least about 50 times greater, at least about 60 times greater, at least about 70 times greater, at least about 80 times greater, at least about 90 times greater, at least about 100 times greater, at least about 200 times greater, at least about 300 times greater, at least about 400 times greater, at least about 500 times greater, at least about 600 times greater, at least about 700 times greater, at least about 800 times greater, at least about 900 times greater, at least about 1000 times greater, at least about 1500 times greater, or at least about 2000 times greater than a level of AKR1C3 (for example, AKR1C3 mRNA or AKR1C3 protein) in a control sample or a control data set.

In some embodiments of a method described herein, detecting in a subject sample a somatic mutation in at least one of the following genes: NFE2L2, KEAP1, or CUL3, comprises sequencing genomic DNA of the subject sample. In some embodiments of a method described herein, detecting in a subject sample a somatic mutation in at least one of the following genes: NFE2L2, KEAP1, or CUL3, comprises sequencing mRNA of the subject sample. In some embodiments, the sequencing is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, and Heliscope single molecule sequencing. In some embodiments, the sequencing comprises performing exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, or Heliscope single molecule sequencing. In some embodiments, the sequencing comprises performing a polymerase chain reaction (PCR). In some embodiments, the PCR is selected from the group consisting of qPCR, RT-PCR, RT-qPCR, and digital PCR.

In some embodiments of a method described herein, if the sequencing detects the somatic mutation in the subject sample, the subject is in need of treatment with the compound of formula (I), or a pharmaceutically acceptable salt thereof.

In some embodiments of a method described herein, the method further comprises sequencing genomic DNA of a control sample. In some embodiments of a method described herein, the method further comprises comparing sequencing data of said sequencing genomic DNA of the subject sample with sequencing data of a control sample or a control data set.

Alternatively, in some embodiments of a method described herein, the method further comprises sequencing mRNA of a control sample. In some embodiments of a method described herein, the method further comprises comparing sequencing data of said sequencing mRNA of the subject sample with sequencing data of a control sample or a control data set.

In some embodiments of a method described herein, the subject sample is characterized by the presence of the somatic mutation by sequencing genomic DNA of the subject sample. In some embodiments of a method described herein, the subject sample is characterized by the presence of the somatic mutation by sequencing mRNA of the subject sample. In some embodiments, the sequencing is selected from the group consisting of exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, and Heliscope single molecule sequencing. In some embodiments, the sequencing comprises performing exome sequencing, targeted genomic sequencing, whole genome sequencing, single-molecule real-time (SMRT) sequencing, ion semiconductor sequencing, pyrosequencing, sequencing by synthesis, combinatorial probe anchor synthesis (cPAS) sequencing, combinatorial probe anchor ligation technology (cPAL) sequencing, SOLiD sequencing, nanopore sequencing, Genap Sys sequencing, Sanger sequencing, Solexa sequencing, DNA nanoball sequencing, or Heliscope single molecule sequencing. In some embodiments, the sequencing comprises performing a polymerase chain reaction (PCR). In some embodiments, the PCR is selected from the group consisting of qPCR, RT-PCR, RT-qPCR, and digital PCR.

In some embodiments of a method described herein, if the sequencing detects the somatic mutation in the subject sample, the subject is in need of treatment with the compound of formula (I), or a pharmaceutically acceptable salt thereof.

In some embodiments of a method described herein, a control sample is characterized by the absence of the somatic mutation by sequencing genomic DNA of the control sample. In some embodiments, a method described herein further comprises comparing sequencing data of said sequencing genomic DNA of the subject sample with sequencing data of a control sample or a control data set.

In some embodiments of a method described herein, a control sample is characterized by the absence of the somatic mutation by sequencing mRNA of the control sample. In some embodiments, a method described herein further comprises comparing sequencing data of said sequencing mRNA of the subject sample with sequencing data of a control sample or a control data set.

In another aspect, the invention provides a use of a AKR1C3 level for selecting a subject for treatment with a compound of formula (I), or a pharmaceutically acceptable salt thereof, wherein a subject is treated with the compound of formula (I), or a pharmaceutically acceptable salt thereof, if a sample of the subject is characterized as having an elevated AKR1C3 level.

In another aspect, the invention provides a use of a somatic mutation for selecting a subject for treatment with a compound of formula (I), or a pharmaceutically acceptable salt thereof, wherein a subject is treated with the compound of formula (I), or a pharmaceutically acceptable salt thereof, if a sample of the subject is characterized by the presence of the somatic mutation and wherein the somatic mutation is detected in one of the following genes: NFE2L2, KEAP1, or CUL3.

In some embodiments of a use described herein, the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof. In some embodiments, the compound of formula (I) is 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof.

### Definitions

For the purpose of interpreting this specification, the following definitions will apply unless specified otherwise and when appropriate, terms used in the singular will also include the plural and vice versa.

As used herein, the term “a”, “an”, “the” and similar terms used in the context of the present invention (especially in the context of the claims) are to be construed to cover both the singular and plural unless otherwise indicated herein or clearly contradicted by the context.

As used herein, the term “(C₁-C₆)alkyl” refers to a straight or branched hydrocarbon chain radical consisting solely of carbon and hydrogen atoms, containing no unsaturation, having from one to six carbon atoms, and which is attached to the rest of the molecule by a single bond. The term “(C₁-C₄)alkyl” is to be construed accordingly. Examples of (C₁-C₆)alkyl include, but are not limited to, methyl, ethyl, n-propyl, 1-methylethyl (iso-propyl), n-butyl, n-pentyl and 1,1-dimethylethyl (t-butyl).

As used herein, the term “(C₂-C₆)alkenyl” refers to a straight or branched hydrocarbon chain radical group consisting solely of carbon and hydrogen atoms, containing at least one double bond, having from two to six carbon atoms, which is attached to the rest of the molecule by a single bond. The term “(C₂-C₄)alkenyl” is to be construed accordingly. Examples of (C₂-C₆)alkenyl include, but are not limited to, ethenyl, prop-1-enyl, but-1-enyl, pent−1-enyl, pent-4-enyl and penta-1,4-dienyl.

As used herein, the term “(C₂-C₆)alkynyl” refers to a straight or branched hydrocarbon chain radical group consisting solely of carbon and hydrogen atoms, containing at least one triple bond, having from two to six carbon atoms, and which is attached to the rest of the molecule by a single bond. The term “(C₂-C₄)alkynyl” is to be construed accordingly. Examples of (C₂-C₆)alkynyl include, but are not limited to, ethynyl, prop-1-ynyl, but-1-ynyl, pent-1-ynyl, pent-4-ynyl and penta-1,4-diynyl.

As used herein, the term “(C₁-C₆)alkoxy” refers to a radical of the formula —ORₐ where Rₐ is a (C₁-C₆)alkyl radical as generally defined above. Examples of (C₁-C₆)alkoxy include, but are not limited to, methoxy, ethoxy, propoxy, isopropoxy, butoxy, isobutoxy, pentoxy, and hexoxy.

As used herein, the term “(C₁-C₆)alkoxy(C₁-C₆)alkyl” refers to a radical of the formula —Rₐ—O—Rₐ where each Rₐ is independently a (C₁-C₆)alkyl radical as defined above. The oxygen atom may be bonded to any carbon atom in either alkyl radical. Examples of (C₁-C₆)alkoxy(C₁-C₆)alkyl include, but are not limited to, methoxy-methyl, methoxy-ethyl, ethoxy-ethyl, 1-ethoxy-propyl and 2-methoxy-butyl.

As used herein, the term “(C₁-C₄)alkylcarbonyl” refers to a radical of the formula —C(═O)—Rₐ where Rₐ is a (C₁-C₄)alkyl radical as defined above.

As used herein, the term “(C₁-C₆)alkylcarbonyl(C₁-C₆)alkyl” refers to a radical of the formula —Rₐ—C(═O)—Rₐ where each Rₐ is independently a (C₁-C₆)alkyl radical as defined above. The carbon atom of the carbonyl group may be bonded to any carbon atom in either alkyl radical.

As used herein, the term “(C₁-C₆)alkoxycarbonyl” refers to a radical of the formula —C(═O)—O—Rₐ where Rₐ is a (C₁-C₆)alkyl radical as defined above.

As used herein, the term “(C₁-C₆)alkoxycarbonyl(C₁-C₆)alkyl” refers to a radical of the formula —Rₐ—C(═O)—O—Rₐ where each Rₐ is independently a (C₁-C₆)alkyl radical as defined above.

As used herein, the term “(C₁-C₄)alkoxycarbonylamino” refers to a radical of the formula —NH—C(═O)—O—Rₐ where Rₐ is a (C₁-C₄)alkyl radical as defined above.

As used herein, the term “hydroxy(C₁-C₆)alkyl” refers to a (C₁-C₆)alkyl radical as defined above, wherein one of the hydrogen atoms of the C₁₋₆alkyl radical is replaced by OH. Examples of hydroxy(C₁-C₆)alkyl include, but are not limited to, hydroxy-methyl, 2-hydroxy-ethyl, 2-hydroxy-propyl, 3-hydroxy-propyl and 5-hydroxy-pentyl.

As used herein, the term “amino(C₁-C₆)alkyl” refers to a (C₁-C₆)alkyl radical as defined above, wherein one of the hydrogen atoms of the (C₁-C₆)alkyl group is replaced by a primary amino group. Representative examples of amino(C₁-C₆)alkyl include, but are not limited to, amino-methyl, 2-amino-ethyl, 2-amino-propyl, 3-amino-propyl, 3-amino-pentyl and 5-amino-pentyl.

As used herein, the term “(C₁-C₄)alkylamino” refers to a radical of the formula —NH—Rₐ where Rₐ is a (C₁-C₄)alkyl radical as defined above.

As used herein, the term “(C₁-C₄)alkylamino(C₁-C₆)alkyl” refers to a radical of the formula —Rₐ₁—NH—Rₐ₂ where Rₐ₁ is a (C₁-C₆)alkyl radical as defined above and Rₐ₂ is a (C₁-C₄)alkyl radical as defined above. The nitrogen atom may be bonded to any carbon atom in either alkyl radical.

As used herein, the term “di(C₁-C₄)alkylamino” refers to a radical of the formula —N(Rₐ)—Rₐ where each Rₐ is a (C₁-C₄)alkyl radical, which may be the same or different, as defined above.

As used herein, the term “di(C₁-C₄)alkylamino(C₁-C₆)alkyl” refers to a radical of the formula —Rₐ₁—N(Rₐ₂)—Rₐ₂ where Rₐ₁ is a (C₁-C₆)alkyl radical as defined above and each Rₐ₂ is a (C₁-C₄)alkyl radical, which may be the same or different, as defined above. The nitrogen atom may be bonded to any carbon atom in any alkyl radical.

As used herein, the term “aminocarbonyl” refers to a radical of the formula —C(═O)—NH₂.

As used herein, the term “aminocarbonylC₁₋₆alkyl” refers to a radical of the formula —Rₐ—C(═O)—NH₂ where Rₐ is a (C₁-C₆)alkyl radical as defined above.

As used herein, the term “(C₁-C₄)alkylaminocarbonyl” refers to a radical of the formula —C(═O)—NH—Rₐ where Rₐ is a (C₁-C₄)alkyl radical as defined above.

As used herein, the term “(C₁-C₄)alkylaminocarbonylC₁₋₆alkyl” refers to a radical of the formula —Rₐ₁—C(═O)—NH—Rₐ₂ where Rₐ₁ is a (C₁-C₆)alkyl radical as defined above and Rₐ₂ is a (C₁-C₄)alkyl radical as defined above.

As used herein, the term “di(C₁-C₄)alkylaminocarbonyl” refers to a radical of the formula —C(═O)—N(Rₐ)—Rₐ where each Rₐ is a (C₁-C₄)alkyl radical, which may be the same or different, as defined above.

As used herein, the term “di(C₁-C₄)alkylaminocarbonylC₁₋₆alkyl” refers to a radical of the formula —Rₐ₁—C(═O)—N(Rₐ₂)—Rₐ₂ where Rₐ₁ is a C₁₋₆alkyl radical as defined above and each Rₐ₂ is a (C₁-C₄)alkyl radical, which may be the same or different, as defined above.

As used herein, the term “(C₃-C₅)cycloalkyl(C₀-C₆)alkyl” refers to a stable monocyclic saturated hydrocarbon radical consisting solely of carbon and hydrogen atoms, having from three to eight carbon atoms, and which is attached to the rest of the molecule by a single bond or by a (C₁-C₆)alkyl radical as defined above. Examples of (C₃-C₅)cycloalkyl(C₀-C₆)alkyl include, but are not limited to, cyclopropyl, cyclopropyl-methyl, cyclobutyl, cyclobutyl-ethyl, cyclopentyl, cyclopentyl-propyl, cyclohexyl, cyclohepty and cyclooctyl.

The term “aryl” refers to 6- to 10-membered aromatic carbocyclic moieties having a single (e.g., phenyl) or a fused ring system (e.g., naphthalene.). A typical aryl group is phenyl group.

As used herein, the term “phenyl(C₀-C₆)alkyl” refers to a phenyl ring attached to the rest of the molecule by a single bond or by a (C₁-C₆)alkyl radical as defined above. Examples of phenyl(C₀-C₆)alkyl include, but are not limited to, phenyl and benzyl.

As used herein, the term “phenyl(C₀-C₆)alkylamino(C₁-C₆)alkyl” refers to a radical of the formula —Rₐ—NH—R_{b} where Rₐ is a (C₁-C₆)alkyl radical as defined above and R_{b} is a phenyl(C₀-C₆)alkyl radical as defined above.

As used herein, the term “phenyl(C₀-C₆)alkylamino((C₁-C₄)alkyl) (C₁-C₆)alkyl” refers to a radical of the formula —Rₐ₁—N(Rₐ₂)—R_{b} where Rₐ₁ is a (C₁-C₆)alkyl radical as defined above, Rₐ₂ is a (C₁-C₄)alkyl radical as defined above and R_{b} is a phenyl(C₀-C₆)alkyl radical as defined above.

As used herein, halo refers to bromo, chloro, fluoro or iodo.

As used herein, the term “halo(C₁-C₆)alkyl” refers to (C₁-C₆)alkyl radical, as defined above, substituted by one or more halo radicals, as defined above. Examples of halogen(C₁-C₆)alkyl include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl and 1,4,4-trifluorobutan-2-yl.

The term “heterocyclyl” refers to a saturated or partially saturated, but not aromatic, ring or ring systems, which include a monocyclic ring, fused rings, bridged rings and spirocyclic rings having the specified number of ring atoms. For example, heterocyclyl includes, but not limited to, 5- to 6-membered heterocyclyl, 4- to 10-membered heterocyclyl, 4- to 14-membered heterocyclyl and 5- to 14-membered heterocyclyl. Unless otherwise specified, the heterocyclyl contain 1 to 7, 1 to 5, 1 to 3, or 1 to 2 heteroatoms independently selected from the group consisting of nitrogen, oxygen and sulphur as ring members, where the N and S can also optionally be oxidized to various oxidation states. The heterocyclic group can be attached at a heteroatom or a carbon atom. Examples of such heterocyclyl include, but are not limited to, azetidine, oxetane, piperidine, piperazine, pyrroline, pyrrolidine, imidazolidine, imidazoline, morpholine, tetrahydrofuran, tetrahydrothiophene, tetrahydrothiopyran, tetrahydropyran, 1,4-dioxane, 1,4 oxathiane, hexahydropyrimidinyl, 3-azabicyclo[3.1.0]hexane, azepane, 3-azabicyclo[3.2.2]nonane, decahydroisoquinoline, 2-azaspiro[3.3]heptane, 2-oxa-6-azaspiro[3.3]heptane, 2,6-diazaspiro[3.3]heptane, 8-aza-bicyclo[3.2.1]octane, 3,8-diazabicyclo[3.2.1]octane, 3-Oxa-8-aza-bicyclo[3.2.1]octane, 8-Oxa-3-aza-bicyclo[3.2.1]octane, 2-Oxa-5-aza-bicyclo[2.2.1]heptane, 2,5-Diaza-bicyclo[2.2.1]heptane, 1,4-dioxa-8-aza-spiro[4.5]decane, 3-oxa-1,8-diazaspiro[4.5]decane, octahydropyrrolo[3,2-b]pyrrol, and the like.

The term “fused heterocyclyl” refers to a heterocyclyl, as defined above, which is fused to an aryl (e.g., phenyl) or a heteroaryl ring as defined above. Examples of such fused heterocyclyl include, but are not limited to, 1,2,3,4-tetrahydroisoquinoline, indoline, isoindoline, 1,2,3,4-tetrahydro-2,7-naphthyridine, 5,6,7,8-tetrahydro-1,7-naphthyridine, 1,2,3,4-tetrahydro-2,6-naphthyridine, 5,6,7,8-tetrahydro-1,6-naphthyridine, 2,3,4,5-tetrahydro-1H-benzo[d]azepine, 1,2,3,4-tetrahydro-1,4-epiminonaphthalene, 2,3-dihydrobenzofurane, 5,6,7,8-tetrahydropyrido[3,4-b]pyrazine, and the like. As used herein, the term “heterocyclyl(C₀-C₆)alkyl” refers to a heterocyclic ring as defined above which is attached to the rest of the molecule by a single bond or by a (C₁-C₆)alkyl radical as defined above.

The term “heteroaryl” refers to aromatic moieties containing at least one heteroatom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, benzopyranyl, benzothiophenyl, benzoimidazolyl, benzoxazolyl, 1H-benzo[d][1,2,3]triazolyl, and the like.). The heteroaromatic moiety may consist of a single or fused ring system. A typical single heteroaryl ring is a 5- to 6-membered ring containing one to three heteroatoms independently selected from oxygen, sulfur and nitrogen and a typical fused heteroaryl ring system is a 9- to 10-membered ring system containing one to four heteroatoms independently selected from oxygen, sulfur and nitrogen. The fused heteroaryl ring system may consist of two heteroaryl rings fused together or a hetereoaryl fused to an aryl (e.g., phenyl). As used herein, the term “heteroaryl(C₀-C₆)alkyl” refers to a heteroaryl ring as defined above which is attached to the rest of the molecule by a single bond or by a (C₁-C₆)alkyl radical as defined above.

Unless specified otherwise, the term “compounds of the present invention” refers to compounds of formula (I), as defined herein, and salts thereof, as well as all stereoisomers (including diastereoisomers and enantiomers), rotamers, tautomers and isotopically labeled compounds (including deuterium substitutions), as well as inherently formed moieties. The term “compounds of the (present) invention” or “a compound of the (present) invention” refers to a compound as defined in any one of the embodiments mentioned below.

### Compounds of Formula (I)

Embodiments of the invention described herein relate, in part, to a compound of Formula (I): or a pharmaceutically acceptable salt thereof:

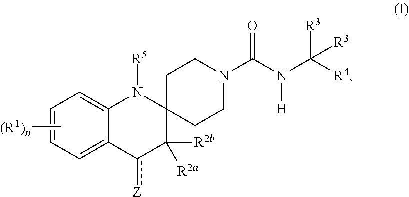

wherein:
[symbol] is a single bond or a double bond;
Z is either OH, when [symbol] is a single bond; or O, when [symbol] is a double bond;
each R¹ is independently selected from the group consisting of, (C₁-C₆)alkyl, (C₁-C₆)alkoxy, (C₀-C₄)alkylN(R′)₂, and halo;
R^{2a} and R^{2b} are each independently selected from the group consisting of H, (C₁-C₆) alkyl, and halo;
each R³ is independently selected from the group consisting of H, and halo;
R⁴ is selected from the group consisting of aryl, a 5 to 6-membered heteroaryl comprising 1, 2, 3, or 4 heteroatoms independently selected from N, O, and S; and a 9 to 10-membered fused bicyclic heteroaryl comprising 1, 2, 3, or 4 heteroatoms independently selected from N, O, and S; wherein any of the foregoing is optionally substituted with one or more R⁶;
R⁵ is selected from the group consisting of H; (C₁-C₆)alkyl; (C₂-C₆)alkenyl; (C₀-C₄)alkylOR⁸; (C₁-C₄)alkyl(C₃-C₁₀)cycloalkyl; halo(C₁-C₆)alkyl; (C₂-C₃)alkynyl; (C₁-C₄)alkylN(R¹⁰)₂;
each R⁶ is independently selected from the group consisting of halo; (C₁-C₆)alkyl; (C₁-C₆)alkoxy; halo(C₁-C₆)alkyl; OH; aryl; 3 to 6-membered heterocycle; 5- to 6-membered heteroaryl; (C₀-C₄)alkylS(O)ₘ(C₁-C₆)alkyl; halo(C₁-C₆)alkoxy; (C₀-C₄)alkylS(O)ₘN(R′)₂; (C₀-C₄)alkyl N(R⁸)₂; (C₀-C₄)alkyl(CO)OR⁷; N(R⁸)S(O)ₘ(C₁-C₆)alkyl; N(R⁸)S(O)ₘ(C₃-C₆)cycloalkyl; OP(O)(OH)₂; (C₀-C₃)alkyl(CO)NHR¹¹; (C₀-C₃)alkylOR⁷, and (C₃-C₁₀)cycloalkyl; wherein each R⁶, when not being halo, OH, or OP(O)(OH)₂, is optionally substituted with one to three R⁹; or two neighboring R⁶, together with the atoms to which they attach form a 5 to 7-membered heterocycle or (C₅-C₈)cycloalkyl;
each R⁷ and R⁸ is independently selected from the group consisting of H or (C₁-C₆)alkyl, that is optionally substituted with one to three R⁹;
each R⁹ is independently selected from the group consisting of halo; —OH; amino, (C₁-C₄)alkylamino, di(C₁-C₄)alkylamino, OP(O)(OH)₂; (C₁-C₆)alkyl; (C₁-C₃)alkynyl; (C₁-C₆)alkoxy; halo(C₁-C₆)alkyl; (C₀-C₄)alkylS(O)ₘ(C₁-C₆)alkyl; halo(C₁-C₆)alkoxy; 3 to 6-membered heterocycle which is optionally substituted with oxo (═O); (C₀-C₄)alkylS(O)ₘN(R¹⁰)₂; (C₀-C₄)alkyl(CO)R¹⁰; (C₀-C₄)alkyl(CO)OR¹⁰; (C₀-C₄)alkylNR¹⁰S(O)ₘ(C₁-C₆)alkyl; (C₀-C₄)alkylOR¹⁰; (C₀-C₄)alkylN(R¹⁰)₂; (C₀-C₄)alkylCN; (C₀-C₄)alkylN(R¹⁰)₂; and (C₀-C₄)alkyl(CO)N(R¹⁰)₂;
each R¹⁰ is independently selected from the group consisting of H, (C₁-C₆)alkyl; or 3 to 6-membered heterocycle, wherein the 3 to 6-membered heterocycle is optionally substituted with one or more of (C₁-C₆)alkyl; and oxo (═O);
each R¹¹ is selected from the group consisting of H; 4 to 6-membered heterocycle which is optionally substituted with one to four R¹²; (C₃-C₆)cycloalkyl which is optionally substituted with one to four R¹²; (C₀-C₃)alkyl(C₃-C₆)cycloalkyl (C₁-C₃)alkyl which is optionally substituted with halo; CH₂-aryl which is optionally substituted with one to three R¹²; (C₁-C₆)alkyl; (C₂-C₆)alkenyl; or (C₂-C₆)alkynyl, wherein each of the (C₁-C₆)alkyl; (C₂-C₆)alkenyl; and (C₂-C₆)alkynyl is optionally substituted with one or more R¹³
each R¹² is independently selected from the group consisting of OH, (C₁-C₃)alkoxy, NH₂; or (C₁-C₃)alkyl optionally substituted with one or more OH;
each R¹³ is independently selected from the group consisting of halo, OH, amino, (C₁-C₄)alkylamino, di(C₁-C₄)alkylamino, (C₁-C₃)alkoxy; and C(O)—(C₃-C₅)cycloalkyl;
m is 0, 1, or 2; and
n is 0, 1 or 2.

In some embodiments, the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof. In some embodiments, the compound of formula (I) is 6′-fluoro-N-(4-fluorobenzyl)-4′-oxo-3′,4′-dihydro-1′H-spiro[piperidine-4,2′-quinoline]-1-carboxamide, or a pharmaceutically acceptable salt thereof. In some embodiments, the compound of formula (I) is N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof. Compounds of formula (I) are described in International Publication No. WO2021/005586, which is incorporated by reference herein. In some embodiments, the compound of formula (I) is a compound of formula (I), or a pharmaceutically acceptable salt thereof, described in International Publication No. WO2021/005586.

### Pharmaceutical Compositions

A compound of formula (I), or a pharmaceutically acceptable salt thereof, described herein, can be a component of a pharmaceutical composition comprising a compound of formula (I), or a pharmaceutically acceptable salt thereof, and one or more pharmaceutically acceptable carriers.

As used herein, the term “pharmaceutical composition” refers to a compound of formula (I), or a pharmaceutically acceptable salt thereof, together with at least one pharmaceutically acceptable carrier, in a form suitable for oral or parenteral administration. In some embodiments, a pharmaceutical composition described herein is suitable for oral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22d Ed. Pharmaceutical Press, 2013, pp. 1049-1070).

The term “a therapeutically effective amount” of a compound of formula (I) refers to an amount of the compound of formula (I) that will elicit the biological or medical response of a subject, for example, reduction or inhibition of an enzyme or a protein activity, or ameliorate symptoms, alleviate conditions, slow or delay disease progression, or prevent a disease. In one non-limiting embodiment, the term “a therapeutically effective amount” refers to the amount of the compound of formula (I) that, when administered to a subject, is effective to (1) at least partially alleviate, inhibit, prevent and/or ameliorate a condition, or a disorder or a disease (i) mediated by KARS, or (ii) disease sensitive to KARS inhibition, or (iii) characterized by activity (normal or abnormal) of KARS; or (2) reduce or inhibit a disease sensitive to KARS inhibition. The invention further provides methods of treating, or preventing diseases and/or disorders related to high AKR1C3 expression or sensitivity to KARS inhibition, comprising administering to a subject in need thereof a therapeutically effective amount of an AKR1C3-dependent KARS inhibitor. In some embodiments, a therapeutically effective amount of a compound of formula (I) is effective, when administered to a subject, to inhibit KARS activity. In some embodiments, inhibition of KARS activity ameliorates disease symptoms, alleviate disease conditions, slows or delays disease progression, or prevents a disease sensitive to KARS inhibition. In some embodiments, a therapeutically effective amount of the compound of formula (I) is effective to reduce the number of cancer cells in a subject; reduce primary tumor size; inhibit or stop cancer cell infiltration into peripheral organs; inhibit tumor metastasis; inhibit or stop tumor growth; and/or relieve to some extent one or more of the symptoms associated with the disease or disorder. In vivo efficacy can, for example, be measured by assessing the duration of survival, time to disease progression (TTP), time to relapse, response rates (e.g. CR and PR), duration of response, and/or quality of life. In some embodiments, in vivo efficacy can, for example, be measured by assessing the enzymatic activity or expression level of a biomarker (for example, an mRNA level or a protein level), for example, a level of AKR1C3. In some embodiments, in vivo efficacy can, for example, be measured by assessing the level of KARS enzymatic activity.

As used herein, the term “subject” refers to primates (e.g., humans, male or female), monkeys, dogs, rabbits, guinea pigs, pigs, rats and mice. In certain embodiments, the subject is a primate. In yet other embodiments, the subject is a human. Unless indicated otherwise, as used herein, the term “subject” is interchangeable with the term “patient.”

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker (for example, reducing a level of a biomarker, for example, an AKR1C3 level) associated with the disease or disorder, including those which may not be discernible to the patient. “Treat,” “treating,” or “treatment” can also refer to modulating the disease or disorder, either physically, (e.g., stabilization of a discernible symptom), physiologically, (e.g., stabilization of a physical parameter), or both. More specifically, “treatment” means any action that results in the improvement or preservation of anatomical function affected by a particular disease or disorder, and/or quality of life in a subject having a disease or disorder. As used herein, “treatment” may mean any manner in which one or more of the symptoms of a disease or disorder are ameliorated or otherwise beneficially altered. As used herein, amelioration of the symptoms of a disease or disorder refers to any lessening, whether permanent or temporary, lasting or transient, that can be attributed to or associated with treatment by the methods of the present invention.

As used herein, the term “prevent”, “preventing” or “prevention” of any disease or disorder refers to the prophylactic treatment of the disease or disorder; or delaying the onset or progression of the disease or disorder, for example, by prophylactic treatment. Prevention can include any action that prevents or slows a worsening in function, quality of life, and/or another parameter associated with a particular disease or disorder in a patient with the particular disease or disorder and at risk for said worsening.

As used herein, a subject is “in need of” a treatment if the subject would benefit biologically, medically, or in quality of life from such treatment.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

A compound of the formula (I) can be in the form of one of the possible stereoisomers, rotamers, atropisomers, tautomers or mixtures thereof, for example, as substantially pure geometric (cis or trans) stereoisomers, diastereomers, optical isomers (antipodes), racemates, or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic compound may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high performance liquid chromatography (HPLC) using a chiral adsorbent.

In some embodiments, a compound of formula (I) is a component of a pharmaceutical composition. For example, described herein is a pharmaceutical composition comprising a compound of formula (I), or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. In some embodiments, a pharmaceutical composition described herein is formulated for oral administration. Topical administration may also pertain to inhalation or intranasal application. Pharmaceutical compositions of formula (I) described herein can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

In some embodiments, a pharmaceutical composition comprising a compound of formula (I), or a pharmaceutically acceptable salt thereof, is administered to a subject (e.g., a patient). Administering can be by any suitable means, including direct delivery to a desired organ, cell or tissue, oral, inhalation, intranasal, intratracheal, buccal, sublingual, intrathecal, intravenous, intramuscular, intra-articular, subcutaneous, intradermal, intraperitoneal, intraspinal, epidural, intradural, subdural, retrobulbar, ophthalmic, intracorneal, conjunctival, intraocular, intravitreal, parenteral, intracranial, intracerebral, intracerebro-ventricular, directly to the lung, and other parental routes of administration. Routes of administration may be combined, if desired. In some embodiments, a pharmaceutical composition comprising a compound of formula (I), or a pharmaceutically acceptable salt thereof, is administered to a subject orally. Dosing can be by any suitable route, e.g. by injections, such as intravenous or subcutaneous injections, depending in part on whether the administration is brief or chronic. In some embodiments, dosing is by an oral route. Various dosing schedules including but not limited to single or multiple administrations over various time-points, bolus administration, and pulse infusion are contemplated herein.

A composition comprising a compound of formula (I), or a pharmaceutically acceptable salt thereof, can be prescribed or administered to a subject (for example, a subject in need of treatment with a compound of formula (I), or a pharmaceutically acceptable salt thereof) at an appropriate dose level. For example, a dose of the composition comprising a therapeutically effective amount of the compound of formula (I), or a pharmaceutically acceptable salt thereof, can be prescribed or administered to the subject. In some embodiments, the dose comprises about 10 mg, about 25 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg about 1200 mg, about 1300 mg, about 1400 mg, or about 1500 mg of the compound of formula (I), or a pharmaceutically acceptable salt thereof. In some embodiments, the dose comprises at least about 10 mg, at least about 25 mg, at least about 50 mg, at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1100 mg at least about 1200 mg, at least about 1300 mg, at least about 1400 mg, or at least about 1500 mg of the compound of formula (I), or a pharmaceutically acceptable salt thereof. In some embodiments, the dose comprises from about 10 mg to about 1500 mg, from about 50 mg to about 1000 mg, from about 50 mg to about 1500 mg, from about 50 mg to about 250 mg, from about 50 mg to about 500 mg, from about 100 mg to about 1000 mg, from about 500 mg to about 1000 mg, from about 250 mg to about 500 mg, from about 500 mg to about 750 mg, from about 750 mg to about 1000 mg, from about 100 mg to about 400 mg, from about 200 mg to about 500 mg, from about 300 mg to about 600 mg, from about 400 mg to about 700 mg, from about 500 mg to about 800 mg, from about 600 mg to about 900 mg, or from about 700 mg to about 1000 mg of the compound of formula (I), or a pharmaceutically acceptable salt thereof. In some embodiments, the dose is administered every 3 hours, every 6 hours, every 8 hours, every 12 hours, every 24 hours, every 2 days, every 3 days, every 4 days, every 5 days, every 6 days, or every week.

In some embodiments a composition comprising a compound of formula (I), or a pharmaceutically acceptable salt thereof, is administered orally.

In some embodiments, a composition comprising a compound of formula (I), or a pharmaceutically acceptable salt thereof, is formulated for oral administration.

### Biomarkers

Described herein are methods of identifying a subject for treatment with a compound of formula (I), or a pharmaceutically acceptable salt thereof, as well as methods of treating a subject with a compound of formula (I), or a pharmaceutically acceptable salt thereof. Methods described herein can include a step of detecting or determining in a subject or a subject sample a level of a biomarker, for example, AKR1C3 protein or nucleic acid, for example, AKR1C3 mRNA transcripts. In some embodiments, an elevated level of AKR1C3 identifies a subject as in need of treatment with the compound of formula (I), or a pharmaceutically acceptable salt thereof. In some embodiments, a method described herein can include a step of detecting in a subject or a subject sample a somatic mutation in one or more genes, for example NFE2L2, KEAP1, or CUL3. In some embodiments, a somatic mutation in one or more genes, for example, a somatic mutation in NFE2L2, KEAP1, or CUL3, is a biomarker. In some embodiments, detecting a somatic mutation of one or more genes identifies a subject as in need of treatment with the compound of formula (I), or a pharmaceutically acceptable salt thereof.

As used herein, the term “determine,” “determining,” or “determination” includes any means of determining, including direct and indirect determination. For example, “determining” can include any means of determining the presence or a level of a biomarker, for example, an AKR1C3 biomarker, for example, a level of AKR1C3 protein or mRNA, in a subject or a subject sample. As used herein, the term “detect,” “detecting,” or “detection” includes any means of detecting, including direct and indirect detection. For example, “detecting” can include any means of detecting the presence or a level of a biomarker, for example, an AKR1C3 biomarker, for example, a level of AKR1C3 protein or mRNA, in a subject or a subject sample. Methods of detecting or determining the presence of or a level of an AKR1C3 protein biomarker include, but are not limited to, antigen detection and quantification assays (for example, Western blot, quantitative Western blotting, immunohistochemistry, immunocytochemistry, enzyme-linked absorbent assay (ELISA), immunoprecipitation, immunoelectrophoresis or dot blot, immunostaining of cells, fluid, tissue or extracts or lysates thereof, and other methods of immunodetection). Methods of detecting or determining the presence of or a level of a AKR1C3 nucleic acid biomarker, for example, mRNA, include, but are not limited to, RNA detection and quantification assays (for example, RNA-Seq (for example, mRNA-Seq), RT-PCR, digital PCR, and RT-qPCR). Methods of detecting or determining the presence of a nucleic acid biomarker (for example, a somatic mutation of an NFE2L2, CUL3, or KEAP1 gene sequence), for example, DNA (for example, genomic DNA), include, but are not limited to methods of sequencing DNA (for example, genomic DNA) (for example, exome sequencing, targeted genomic sequencing, whole genome sequencing, SMRT sequencing, ion semiconductor sequencing, pyrosequencing, sequencing by synthesis, cPAS sequencing, cPAL sequencing, SOLiD sequencing, nanopore sequencing, Genap Sys sequencing, Sanger sequencing, Solexa sequencing, DNA nanoball sequencing, Heliscope single molecule sequencing, PCR, qPCR, digital PCR, and Sanger sequencing).

The term “biomarker” as used herein refers to an indicator, e.g. predictive, diagnostic, and/or prognostic, which can be detected in a sample, e.g., a particular gene (including, but not limited to, alterations in gene sequence (for example, a somatic mutation) relative to a wild type sequence or alterations in gene expression levels (for example, as determined by mRNA transcripts of a gene) relative to a control sample or a control data set) or protein (including, but not limited to, alterations in protein expression levels relative to a control sample or a control data set) encoded by said gene. Biomarkers can include or one or more somatic mutations of a gene, for example, NFE2L2, CUL3, or KEAP1. The biomarker may serve as an indicator of a particular disease or disorder or a particular subtype of disease or disorder (e.g., cancer) characterized by certain molecular, pathological, histological, and/or clinical features (e.g. responsiveness to treatment with a compound of formula (I), or a pharmaceutically acceptable salt thereof). In some embodiments, a biomarker is a collection of genes or proteins (e.g. single or multiple gene and protein expression levels) or a collective number of mutations/alterations (e.g. somatic mutations) in a collection of genes. Biomarkers include, but are not limited to, polynucleotides, polynucleotide alterations (e.g., gene sequence mutations, for example, somatic mutations), polypeptides, and proteins. In some embodiments described herein, the biomarker is an AKR1C3 protein level. In some embodiments described herein, the biomarker is an AKR1C3 nucleotide sequence expression level. In some embodiments described herein, the biomarker is an AKR1C3 gene expression level. In some embodiments described herein, the biomarker is an AKR1C3 mRNA transcript level. In some embodiments described herein, the biomarker is a NFE2L2, CUL3, or KEAP1 gene sequence. In some embodiments described herein, the biomarker is a somatic mutation in a NFE2L2, CUL3, or KEAP1 gene sequence.

The term “level” refers to the presence or amount of a biomarker in a sample, for example, a subject sample or a control sample, or a data set, for example, a control data set.

“Increased level”, “increased levels”, “elevated level”, “elevated levels”, or “high levels” of a biomarker refers to an increased level of a biomarker (for example, a protein or mRNA biomarker) in a sample (for example, a subject sample) relative to a control sample, such as an individual or individuals who are not suffering from the disease or disorder (e.g. cancer) or a control data set, such as a data set comprised of biomarker levels from an individual or individuals who are not suffering from the disease or disorder (e.g. cancer). In some embodiments, increased levels of a biomarker are detectable in the subject or a subject sample.

“Decreased level”, “decreased levels”, “reduced level”, “reduced levels”, or “low levels” of a biomarker refers to a decreased level of a biomarker (for example, a protein or mRNA biomarker) in a sample (for example, a subject sample) relative to a control sample, such as an individual or individuals who are not suffering from the disease or disorder (e.g. cancer) or a control data set, such as a data set comprised of biomarker levels from an individual or individuals who are not suffering from the disease or disorder (e.g. cancer). In some embodiments, increased levels of a biomarker are detectable in the subject or a subject sample.

The terms “level of expression” or “expression level” in general are used interchangeably and generally refer to the amount of a biomarker in a biological sample. “Expression” generally refers to the process by which information (e.g. gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g. posttranslational modification of a polypeptide). Fragments of the transcribed polynucleotide, the translated polypeptide, or polynucleotide and/or polypeptide modifications (e.g. posttranslational modification of a polypeptide) shall also be regarded as expressed whether they originate from a transcript generated by alternative splicing or a degraded transcript, or from a post-translational processing of the polypeptide, e.g. by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol.51:263 (1987) and Erlich, ed., PCR Technology, (Stockton Press, NY, 1989). As used herein, PCR is considered to be one, but not the only, example of a nucleic acid polymerase reaction method for amplifying nucleic acid, for example, nucleic acid in a sample (for example, a control sample or a subject sample), comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid. In some embodiments, PCR is used to detect a somatic mutation (for example, a somatic mutation of KEAP1, NFE2L2, or CUL3) or to detect or determine the level of expression of a gene of interest (for example, KEAP1, NFE2L2, or CUL3).

The term “multiplex-PCR” refers to a single PCR reaction carried out on nucleic acids obtained from a single source (e.g., an individual sample from a subject) using more than one primer set for the purpose of amplifying two or more DNA sequences in a single reaction.

The term “quantitative PCR” (also called “qPCR” or “real-time PCR”) refers to a PCR reaction used to monitor the amplification of a targeted nucleic acid species. qPCR generally relies upon fluorescent dyes that intercalate with double-stranded DNA or fluorescently labeled sequence-specific DNA probes that can hybridize with a PCR product of interest in order to detect and quantify amplification of a nucleic acid species of interest. qPCR can be quantitative or semi-quantitative.

“Reverse transcription PCR” or “RT-PCR” refers to a form of PCR in which an RNA template (for example, an mRNA transcript) is converted into a complementary DNA (cDNA) using a reverse transcriptase. The cDNA produced by this reaction is then amplified by PCR.

“Reverse transcription quantitative PCR” or “RT-qPCR” refers to a form of qPCR that is quantitative or semi-quantitative. In general, RT-qPCR relies upon the same methodology as qPCR, but employs a reverse transcriptase to produce a cDNA from mRNA. RT-qPCR is used to monitor the amplification of a targeted mRNA species, allowing quantification of mRNA in a sample.

“Digital PCR” refers to a PCR method used to directly quantify and clonally amplify nucleic acids strands including DNA, cDNA, or RNA. Digital PCR carries out a single PCR reaction within a sample, similar to traditional PCR. However, as compared to traditional PCR, the sample is separated into a large number of partitions (for example, 10⁴ partitions of a single sample), and the PCR reaction is carried out in each partition individually.

The term “diagnosis” is used herein to refer to the identification or classification of a molecular or pathological state, disease or condition (e.g. cancer). For example, “diagnosis” may refer to identification of a particular type of cancer. “Diagnosis” may also refer to the classification of a particular subtype of cancer, for instance, by histopathological criteria, or by molecular features (e.g. a subtype characterized by expression of one or a combination of biomarkers (e.g. particular genes or proteins encoded by said genes)).

The term “sample” or “biological sample”, as used herein, refers to a composition that is obtained or derived from a subject and/or individual of interest that contains a cellular, fluid, and/or other molecular entity that is to be characterized and/or identified, for example, based on physical, biochemical, chemical, and/or physiological characteristics. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor cells, tumor lysates, and tissue culture medium, tissue extracts such as homogenized tissue, tumor tissue, cellular extracts, and combinations thereof. In some embodiments, a sample is a tissue sample, a blood sample, or a cell sample. In some embodiments, a sample, is or comprises protein or genetic material. In some embodiments, a sample comprises a cell genome, transcriptome, or proteome, for example, a tumor cell genome, transcriptome, or proteome.

By “tissue sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g. tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like. In some instances, the tissue sample or tumor tissue sample is not a blood sample or sample or a blood constituent, such as plasma. In a preferred embodiment, the tissue sample or cell sample is a tumor sample.

A “subject sample,” as used herein can be a sample, for example, a biological sample, from or derived from a subject, for example, a subject in need of treatment or prophylaxis. A subject sample can include any sample obtained from a subject of interest that would be expected or is known to contain the cellular and/or molecular entity that is to be characterized, for example, a biomarker. In some embodiments, a subject sample includes components not directly from the subject. For example, a subject sample can be a mixture of material directly from a subject (for example, a subject's tissue) or derived from a subject (for example, a cell line derived from a subject's tissue) and additional material(s) (for example, a buffer or a cell culture fluid). A subject sample can be comprised of one or more cells, cell populations, cell lysates, tissues, or fluids of the subject, for example, one or more cells, cell populations, cell lysates, tissues, or fluids obtained from the subject or provided by the subject, originating from the subject. In some embodiments, a subject sample can include one or more cancerous cells, for example, one or more tumor cells (for example, liquid tumor cells or solid tumor cells); whole blood; plasma; serum; blood-derived cells; platelets; lymphatic fluid; urine; feces; mucus; sputum; sweat; saliva; semen; cerebrospinal fluid; bone marrow; amniotic fluid; one or more tissue samples; primary or cultured cells or cell lines; cell supernatants; cell lysates; vitreous fluid; synovial fluid; follicular fluid; milk; tears; tumor lysates; tissue culture medium; tissue extracts such as homogenized tissue; tumor tissue; cellular extracts; and combinations thereof. In some embodiments, a subject sample, is or comprises protein or genetic material from or derived from a subject. For example, in some embodiments, a subject sample, is or comprises a genome, transcriptome, or proteome from or derived from a subject, for example, a genome, transcriptome, or proteome of a tumor cell of a subject.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein. A tumor cell can be a liquid tumor cell or a solid tumor cell.

A “control sample,” as used herein, refers to a sample, tissue, cell, data set, standard, or level that is used for comparison purposes. In one embodiment, a control sample, is obtained from a healthy and/or non-diseased part of the body (e.g. tissue or cells) of the same subject or individual. For example, the control sample, can be healthy and/or non-diseased tissue or cells adjacent to the diseased tissue or cells (e.g. tissue or cells adjacent to a tumor). In another embodiment, a control sample is obtained from a healthy and/or non-diseased part of the body (e.g. tissues or cells) of an individual who is not the same subject or individual. In some embodiments, a control sample is or comprises a genome, transcriptome, or proteome of a control cell, tissue, or fluid.

A “control data set,” as used herein, refers to a data set comprising one or more samples, tissues, cells, standards, or levels that is used for comparison purposes, for example, for comparison to a subject sample. In one embodiment, a control data set, is comprised of data obtained from a healthy and/or non-diseased part of the body (e.g. tissue or cells) of the same subject or individual. In some embodiments, a control data set is comprised of data obtained from one or more healthy and/or non-diseased individuals who are not the subject. In some embodiments, a control data set includes data regarding the level (for example, protein or RNA level) of one or more biomarkers in one or more healthy control individuals. In some embodiments, a control data set includes data regarding the presence, absence, or prevalence of one or more biomarkers (for example, the presence, absence, or prevalence of one or more disease-linked nucleotide sequences or somatic mutations) in one or more healthy control individuals. In some embodiments, a control data set includes genomic, transcriptomic, or proteomic data.

The term “AKR1C3” as used herein refers to a protein encoded by a Aldo-Keto Reductase Family 1 Member C3 gene sequence (also known as DD3; DDX; PGFS; HAKRB; HAKRe; HA1753; HSD17B5; or hluPGFS), described, for example, in NCBI Gene ID: 8644, and its orthologs, or a nucleotide sequence (for example, an mRNA sequence) or gene sequence encoding said protein.

The term “KEAP1” as used herein refers to a protein encoded by A Kelch Like ECH Associated Protein 1 gene sequence (also known as INFE2L2; or KLHL19), described, for example, in NCBI Gene ID: 9817, and its orthologs, or a nucleotide sequence (for example, an mRNA sequence) or gene sequence encoding said protein.

The term “CUL3” as used herein refers to a protein encoded by a Cullin 3 gene sequence (also known as CUL-3; PHA2E; or NEDAUS), described, for example, in NCBI Gene ID: 8452, and its orthologs, or a nucleotide sequence (for example, an mRNA sequence) or gene sequence encoding said protein.

The term “NFE2L2” as used herein refers to a protein encoded by a NFE2 Like Bzip Transcription Factor 2 gene sequence (also known as NRF2; HEBP1; Nrf-2; or IMDDHH), described, for example, in NCBI Gene ID: 4780, and its orthologs, or a nucleotide sequence (for example, an mRNA sequence) or gene sequence encoding said protein.

The term “KARS” as used herein refers to a protein encoded by a Lysyl-Trna Synthetase 1 gene sequence (also known as KRS; KARS; KARS2; LEPID; CMTRIB; DEAPLE; or DFNB89), described, for example, in NCBI Gene ID: 3735, and its orthologs, or a nucleotide sequence (for example, an mRNA sequence) or gene sequence encoding said protein.

### Genetic Mutations

Described herein are methods of identifying a subject in need of treatment with a compound of formula (I), or a pharmaceutically acceptable salt thereof. In some embodiments, a method described herein includes the step of detecting in a subject sample a somatic mutation, for example, detecting a somatic mutation in at least one of the following genes: NFE2L2, KEAP1, or CUL3A. Somatic mutations include genetic sequences that result in an increased likelihood of an individual carrying the somatic mutation of developing or being predisposed to developing a particular disease. For example, an individual carrying a somatic mutation may have an increased likelihood of developing or being predisposed to developing a cancer. Somatic mutations can be associated with a disease through genomic studies, for example, genome-wide association studies (GWAS).

Various types of genetic mutations are known in the art and include, for example, point mutations, single nucleotide polymorphisms (SNPs), substitutions, missense, nonsense, frameshift, nucleotide repeat expansions, inversions, insertions, deletions, copy number variations, amplifications, gene duplications, somatic, germline, homozygous, heterozygous, chromosomal rearrangements, splice-site, gain-of-function, hypomorphic, and neomorphic mutations. In some embodiments, the somatic mutation is a point mutation, a single nucleotide polymorphism (SNP) mutation, a substitution mutation, a missense mutation, a nonsense mutation, a frameshift mutation, a nucleotide repeat expansion mutation, an inversion mutation, an insertion mutation, a deletion mutation, a copy number variation mutation, an amplification mutation, a gene duplication mutation, a somatic mutation, a homozygous mutation, a heterozygous mutation, a chromosomal rearrangement mutation, a splice-site mutation, a gain-of-function mutation, a hypomorphic mutation, or a neomorphic mutation in the gene sequence of at least one of the following genes: NFE2L2, KEAP1, or CUL3A. Somatic mutations can occur in any part of a gene sequence, including, for example, protein coding regions, gene enhancers, exons, introns, promoters, splice sites, 5′ UTRs, or 3′ UTRs. In some embodiments, the somatic mutation comprises an amplification of the NFE2L2 gene sequence, or a portion thereof. In some embodiments, the somatic mutation comprises deletion of the KEAP1 or CUL3 gene sequence, or a portion thereof.

Disease-linked nucleotide sequences of KEAP1 are described, for example, in Campbell et al., (2016) Nature Genetics, 48:607-16; Chen, R. (2020) “Cullin 3 and Its Role in Tumorigenesis” in Cullin-RING Ligases and Protein Neddylation, 187-210; Collisson et al., (2014) Nature, 511:543-550; Delgobo et al., (2021) Freed Radical Biology and Medicine, 177:58-71; Gong et al., (2020) Cell Communication and Signaling, 18, 98; Hammerman et al., (2012) Nature, 489:519-525; Hayes and McMahon (2009) Trends in Biochemical Sciences, 34(4):176-88; Jin et al., (2021) Cancer Medicine, 10(23):8673-92; Kandoth et al., (2013) Nature, 502:333-339; Konstantinopoulos et al. (2011) Cancer Res, 71:5081-5089; Ohta et al. (2008) Cancer Res, 68:1303-1309; Padmanabhan et al., (2006)Mol Cell, 21:689-700; Romero et al., (2020) Nature Cancer, 1(6):589-602; Saleh et al., (2021) Journal of Thoracic Oncology, 17(1): 76-88; Shibata et al., (2008) Gastroenterology, 135:1358-1368; Shibata et al. (2011) Neoplasia, 13:864-873; Singh et al., (2006) PLoS Med, 3:e420; Taguchi and Yamamoto (2017) Frontiers in Oncology, 7:85; Wang et al., (2020) “CRL3s: The BTB-CUL3-RING E3 Ubiquitin Ligases” in Cullin-RING Ligases and Protein Neddylation, 211-223; and Yoo et al., (2012) Histopathology, 60:943-952, which are incorporated by reference herein.

Disease-linked nucleotide sequences of CUL3 are described, for example, in Campbell et al., (2016) Nature Genetics, 48:607-16; Chen, R. (2020) “Cullin 3 and Its Role in Tumorigenesis” in Cullin-RING Ligases and Protein Neddylation, 187-210; Collisson et al., (2014) Nature, 511:543-550; Delgobo et al., (2021) Freed Radical Biology and Medicine, 177:58-71; Hammerman et al., (2012) Nature, 489:519-25; Jin et al., (2021) Cancer Medicine, 10(23):8673-92; Ooi et al., (2013) Cancer Res, 73:2044-51; and Wang et al., (2020) “CRL3s: The BTB-CUL3-RING E3 Ubiquitin Ligases” in Cullin-RING Ligases and Protein Neddylation, 211-23, which are incorporated by reference herein.

Disease-linked nucleotide sequences of NFE2L2 are described, for example, in Campbell et al., (2016) Nature Genetics, 48:607-16; Chen, R. (2020) “Cullin 3 and Its Role in Tumorigenesis” in Cullin-RING Ligases and Protein Neddylation, 187-210; Collisson et al., (2014) Nature, 511:543-50; Delgobo et al., (2021) Freed Radical Biology and Medicine, 177:58-71; Goldstein et al., (2016) Cell Rep, 16:2605-2617; Hammerman et al., (2012) Nature, 489:519-25; Jin et al., (2021) Cancer Medicine, 10(23):8673-92; Ooi et al., (2013) Cancer Res, 73:2044-51; Shibata et al., (2011) Neoplasia, 13:864-873; and Wang et al., (2020) “CRL3s: The BTB-CUL3-RING E3 Ubiquitin Ligases” in Cullin-RING Ligases and Protein Neddylation, 211-23, which are incorporated by reference herein.

“Subject tumor genome” as used herein refers to the complete set of genetic information included in cells of a subject tumor, including coding and noncoding portions of chromosomal DNA. A subject tumor genome can include genetic mutations associated with a disease or disorder. For example, in some embodiments of a method described herein, the subject tumor genome comprises a somatic mutation associated with a disease or disorder, for example a cancer. For example, in some embodiments, the subject tumor genome comprises a somatic mutation in one or more of the NFE2L2, KEAP1, or CUL3 gene sequences. In some embodiments, the presence of the somatic mutation in the subject tumor genome can indicate that the subject is in need of treatment with a compound of formula (I), or a pharmaceutically acceptable salt thereof. In some embodiments, a subject tumor genome can include genetic mutations not known to be associated with a disease or disorder, for example, a cancer, but which can indicate that the subject is in need of treatment with a compound of formula (I), or a pharmaceutically acceptable salt thereof.

### Disease and Cancer

Described herein are methods of treating a subject or identifying a subject for treatment with a compound of formula (I), or a pharmaceutically acceptable salt thereof. In particular embodiments, the subject is diagnosed with, suffering from, or predisposed to developing a particular disease or disorder. Diseases and disorders described herein can include conditions that would benefit from treatment including, but not limited to, chronic and acute disorders or diseases including those pathological conditions which predispose a subject to the disease or disorder in question. In particular embodiments, the subject is diagnosed with, suffering from, or predisposed to developing a cancer.

The terms “cancer” and “cancerous” refer to or describe the physiological condition that is typically characterized by unregulated cell growth. Included in this definition are benign and malignant cancers. By “early stage cancer” or “early stage tumor” is meant a cancer that is not invasive or metastatic or is classified as a stage I or II cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. Examples of a cancer also include, but are not limited to, a lung cancer (e.g. a non-small cell lung cancer (NSCLC)), a kidney cancer (e.g. a kidney urothelial carcinoma or RCC), a bladder cancer (e.g. a bladder urothelial (transitional cell) carcinoma (e.g. locally advanced or metastatic urothelial cancer, including 1L or 2L+ locally advanced or metastatic urothelial carcinoma), a breast cancer, a colorectal cancer (e.g. a colon adenocarcinoma), an ovarian cancer, a pancreatic cancer, a gastric carcinoma, an esophageal cancer, a mesothelioma, a melanoma (e.g. a skin melanoma), a head and neck cancer (e.g. a head and neck squamous cell carcinoma (HNSCC)), a thyroid cancer, a sarcoma (e.g. a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g. an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g. a Hodgkin lymphoma or a non-Hodgkin lymphoma (NHL)), a myeloma (e.g. a multiple myeloma (MM)), a mycosis fungoides, a Merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor. More particular examples of such cancers include early stage I-III resectable and unresectable (Stage IIIC) or metastatic (Stage IV) melanoma, lung cancer, including NSCLC, squamous cell cancer (e.g. epithelial squamous cell cancer), lung cancer including small-cell lung cancer (SCLC), and adenocarcinoma of the lung and squamous carcinoma of the lung. In particular examples, the lung cancer is NSCLC, for example a locally advanced or metastatic NSCLC (e.g. stage IIIB NSCLC, stage IV NSCLC, or recurrent NSCLC). Other examples include cancer of the peritoneum, hepatocellular cancer, bladder cancer (e.g. urothelial bladder cancer (e.g. transitional cell or urothelial carcinoma, non-muscle invasive bladder cancer, muscle-invasive bladder cancer, and metastatic bladder cancer) and non-urothelial bladder cancer), gastric or stomach cancer including gastrointestinal cancer, pancreatic cancer, glioblastoma, cervical cancer, ovarian cancer, liver cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycosis fungoides, testicular cancer, esophageal cancer, tumors of the biliary tract, as well as head and neck cancer and hematological malignancies. In some embodiments, the subject is diagnosed with, suffering from, or predisposed to developing a disease or disorder selected from the group consisting of non-small cell lung cancer, lung adenocarcinoma, lung squamous cell carcinoma, a bladder cancer (for example, bladder urothelial carcinoma), a cervical cancer (for example, cervical squamous cell carcinoma), a uterine cancer (for example, uterine endometrial carcinoma), an esophageal cancer (for example, esophageal squamous cell carcinoma), a head and neck cancer (for example, head and neck squamous cell carcinoma), a kidney cancer (for example, papillary renal cell carcinoma), a breast cancer, colorectal cancer, a melanoma, a stomach cancer, castration-resistant prostate cancer (CRPC), T-cell acute lymphoblastic leukemia (T-ALL), acute myeloid leukemia (AML), myelodysplastic syndrome (MDS), and a liver cancer (for example, hepatocellular carcinoma). In particular embodiments, the subject is diagnosed with, suffering from, or predisposed to developing NSCLC. In some embodiments, the subject is diagnosed with, suffering from, or predisposed to developing a squamous cell carcinoma subtype of NSCLC. In some embodiments, the subject is diagnosed with, suffering from, or predisposed to developing an adenocarcinoma subtype of NSCLC.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein. In some embodiments, the tumor is a solid tumor. In some embodiments, the tumor is a liquid tumor.

Subjects who may benefit from treatment with a compound of formula (I), or a pharmaceutically acceptable salt thereof, include those who respond to a therapeutically effective amount of the compound. For example, subjects who may benefit from treatment with a compound of formula (I), or a pharmaceutically acceptable salt thereof, include those subjects who respond or are likely to respond to treatment, for example, subjects who as a result of treatment experience or are likely to experience: an improvement or preservation of anatomical function affected by a particular disease or disorder; improvement in quality of life related to improvement of disease state; alleviation or amelioration of the disease or disorder; alleviation or amelioration of at least one physical parameter or biomarker associated with the disease or disorder; and/or amelioration or improvement in one or more of the symptoms of a disease or disorder.

Described herein are methods for identifying a subject for treatment with a compound of formula (I), or a pharmaceutically acceptable salt thereof. A subject for treatment can be identified by diagnosing the subject as suffering from or predisposed to developing a particular disease or disorder. For example, a subject for treatment can be identified by diagnosing the subject as suffering from or predisposed to developing a cancer, for example, NSCLC. A subject for treatment can be identified by detecting the presence or elevated level of a biomarker. For example, a subject for treatment can be identified by detecting elevated levels of AKR1C3. For example, a subject for treatment can be identified by elevated levels of AKR1C3 in a subject sample relative to a level of AKR1C3 in a control sample, for example, a control subject sample or a control data set. In some embodiments, a subject for treatment can be identified by detecting the presence of one or more somatic mutation in at least one of the following genes: NFE2L2, KEAP1, or CUL3, in a subject sample, for example, a subject tumor genome.

### Immunohistochemistry

In some embodiments of a method described herein, a sample, for example, a subject sample or a control sample, is characterized by a biomarker level, for example, an AKR1C3 protein level, by an antigen detection assay. In some embodiments, the antigen detection assay is an immunohistochemistry (IHC) assay. In general, IHC relies upon detecting the presence, quantity, and/or relative amount of a species of interest using an antigen-detecting compound, for example, an antibody. In some embodiments described herein, an antigen IHC assay includes a step of probing a sample (for example, a control sample or a subject sample) with an antibody capable of binding to a biomarker, for example, AKR1C3 protein.

Antigen-based detection assays generally determine the presence, level, or distribution of a target molecule (for example, a biomarker) in a sample by detecting interaction of the target molecule with a specific binding agent, such as an antibody, that can be detected. For example, a sample is contacted with an antibody (or other binding agent such as an antibody fragment) under conditions permitting antibody-antigen binding. Antibody-antigen binding can be detected by means of a detectable label conjugated to the antibody (direct detection) or by means of a detectable label conjugated to a secondary antibody, which binds specifically to the primary antibody (e.g., indirect detection).

IHC utilizes antibodies or derivatives thereof or other proteinaceous binding agents to analyze histological tissues under the microscope. IHC can include steps of: blocking tissue with reagents to block endogenous sources of nonspecific staining such as (i) enzymes, (ii) endogenous peroxidase, (iii) free aldehyde groups, (iv) immunoglobulins, and other irrelevant molecules that can mimic specific staining; incubating tissue with permeabilization buffer to facilitate penetration of antibodies and other staining reagents into the tissue; incubating tissue with one or more primary antibodies; rinsing the tissue with wash buffer; incubating the tissue with one or more secondary antibodies that bind to one of the one or more primary antibodies; rinsing with wash buffer; and incubating the tissue with detection reagents. The present invention is not limited to this IHC protocol.

In some embodiments of a method described herein, determining a biomarker level, for example, a biomarker level of a subject sample or a control sample, can include producing a biomarker signal intensity score (for example, a biomarker signal intensity score for an AKR1C3 protein level). The IHC signal intensity score of a subject sample or the increase in the IHC signal intensity score of a subject sample relative to the IHC signal intensity score of a control sample can indicate that a subject is in need of treatment with a compound of formula (I), or a pharmaceutically acceptable salt thereof. In some embodiments, if the IHC signal intensity score for the subject sample is at least about 10% greater, at least about 20% greater, at least about 30% greater, at least about 40% greater, at least about 50% greater, at least about 60% greater, at least about 70% greater, at least about 80% greater, at least about 90% greater, or at least about 100% greater than the IHC signal intensity score for the control sample, then the subject is in need of treatment with the compound of formula (I), or a pharmaceutically acceptable salt thereof.

In some embodiments, the IHC signal intensity score can range from 0 to 3, where a score of 0 indicates no detectable signal and a score of 3 indicates strong detectable signal. In some embodiments, this scoring system is referred to as an H-score. The H-score is the sum of: the percentage of strongly staining nuclei multiplied by a factor of 3, the percentage of moderately staining nuclei multiplied by a factor of 2, and the percentage of weakly staining nuclei; this sum is divided by 100. In some embodiments, if the H-score for the subject sample is 0.5 or greater, 1.0 or greater, 1.5 or greater, 2 or greater, 2.5 or greater, 2.6 or greater, 2.7 or greater, 2.8 or greater, or 2.9 or greater, then the subject is in need of treatment with the compound of formula (I), or a pharmaceutically acceptable salt thereof.

In some embodiments, the IHC signal intensity score can range from 0 to 300, where a score of 0 indicates no detectable signal and a score of 300 indicates strong detectable signal. In some embodiments, this scoring system is referred to as an H-score. The H-score is the sum of the percentage of strongly staining nuclei multiplied by a factor of 3, the percentage of moderately staining nuclei multiplied by a factor of 2, and the percentage of weakly staining nuclei. In some embodiments, if the H-score for the subject sample is 50 or greater, 100 or greater, 150 or greater, 200 or greater, 250 or greater, 260 or greater, 270 or greater, 280 or greater, or 290 or greater, then the subject is in need of treatment with the compound of formula (I), or a pharmaceutically acceptable salt thereof.

IHC signal intensity score can be determined based on quantification of detectable signal from an IHC detection reagent. For example, IHC signal intensity score can be determined based on quantification of detectable fluorescent signal or chromogen signal (for example, DAB, 3-Amino-9-ethylcarbazole (AEC), 5-bromo-4-chloro-3-indolyl phosphate: tetranitroblue tetrazolium (BCIP:TNBT), 5-bromo-4-chloro-3-indolyl phosphate: p-nitroblue tetrazolium chloride (BCIP:NBT), or 3,3′, 5,5;-tetramethylbenzidine (TMB), Fast Red, Permanent Red), or signal intensity thereof. Chromogen signal may be produced through a chemical reaction with a suitable enzyme, for example, HRP, glucose oxidase, or alkaline phosphatase. The enzyme can be conjugated to an antibody (for example, a primary antibody or a secondary antibody) or a probe (for example, a streptavidin probe). Fluorescent signal can be generated directly from a protein, for example, green fluorescent protein or red fluorescent protein. Fluorescent signal can also be produced from a suitable quantum dot species, dye, or fluorophore, for example, Alexa Fluor 350, Alexa Fluor 405, Alexa Fluor 488, Alexa Fluor 532, Alexa Fluor 546, Alexa Fluor 555, Alexa Fluor 561, Alexa Fluor 568, Alexa Fluor 594, Alexa Fluor 647, Alexa Fluor 660, Alexa Fluor 680, Alexa Fluor 700, Alexa Fluor 750, BODIPY FL, Coumarin, Cy3, Cy5, Fluorescein (FITC), Oregon Green, Pacific Blue, Pacific Green, Pacific Orange, PE-Cyanine7, PerCP-Cyanine5.5, Tetramethylrhodamine (TRITC), Texas Red, eFluor 450, eFluor 506, eFluor 660, PE-eFluor 610, PerCP-eFluor 710, APC-eFluor 780, Super Bright 436, Super Bright 600, Super Bright 645, Super Bright 702, Super Bright 780 Qdot 525, Qdot 565, Qdot 605, Qdot 655, Qdot 705, or Qdot 800. A quantum dot or fluorophore can be conjugated to an antibody (for example, a primary antibody or a secondary antibody) or a probe (for example, a streptavidin probe). In some embodiments, a sample is counterstained with suitable agent, for example, eosin, hematoxylin, or a suitable DNA binding agent (for example, 4′,6-diamidino-2-phenylindole (DAPI), propidium iodide, SYTO 9, SYTOX Green, or TO-PRO-3).

IHC signal intensity can be calculated based on images captured using a suitable microscope (for example, a brightfield, fluorescent, or confocal microscope) or slide scanner and camera. IHC signal intensity can be quantified from such images using a suitable software program, for example, ImageJ Fiji, ImageScope, Ilastik, Cell Profiler, inForm Image Analysis Software, or IHC Profiler.

The inventors of the disclosed invention, have unexpectedly found that AKR1C3 levels can vary dramatically between individual tumor cells within a single tumor. The inventors have also found that IHC has properties that make it particularly well-suited for use in connection with methods of treating a subject with a compound of formula (I), or a pharmaceutically acceptable salt thereof, identifying a subject for treatment with a compound of formula (I), or a pharmaceutically acceptable salt thereof, and selecting a compound of formula (I), or a pharmaceutically acceptable salt thereof, for treating a subject, where the method requires determining a level of AKR1C3 in a subject sample, characterizing the subject sample as having an elevated level of AKR1C3, or where a subject sample is characterized as having an elevated level of AKR1C3. Thus, the inventors have unexpectedly found that IHC is particularly advantageous for determining a level of AKR1C3 in a sample (for example, a subject sample) in connection with: a method of identifying a subject for treatment with a compound of formula (I), or a pharmaceutically acceptable salt thereof; selecting a compound of formula (I), or a pharmaceutically acceptable salt thereof, for treating a subject; a method of treating a subject that includes a step of determining in a subject sample a level of AKR1C3; a method of treating a subject by administering to the subject an effective amount of a compound of formula (I), or a pharmaceutically acceptable salt thereof, wherein prior to said administering, a subject sample is characterized as having an elevated level of AKR1C3; and a method of treating a subject with a compound of formula (I), or a pharmaceutically acceptable salt thereof, wherein a subject sample is characterized as having an elevated level of AKR1C3.

Notably, IHC allows assessing or scoring the presence or level of a protein or other detectable marker in individual cells. While IHC can be used to produce an overall score for detection of a protein or other detectable marker in a tissue sample (for example, a tumor sample), it provides the advantage of basing that score on assessment of individual cells. These properties of IHC make it particularly advantageous for assessing expression of a protein that shows highly heterogeneous levels of expression in individual cells of a tumor. Thus, because AKR1C3 levels vary dramatically between individual tumor cells, IHC is particularly well-suited for determining a level of AKR1C3 in a subject sample and/or producing an H-score suitable for identifying a subject for treatment with a compound of formula (I), or a pharmaceutically acceptable salt thereof.

### Kits

Also described herein are kits suitable for performing a method described herein or for a use described herein. A kit described herein can comprise components, including, but not limited to, instructions for use, one or more containers for storing the kit components, and/or a pharmaceutically acceptable solution formulated for oral administration. A kit described herein can further include components suitable for: determining in a subject sample a level of a biomarker, for example, AKR1C3; characterizing a subject sample as having an elevated biomarker level (for example, a AKR1C3 biomarker level); identifying a subject for treatment with a compound of formula (I), or a pharmaceutically acceptable salt thereof, detecting in a subject sample a somatic mutation in at least one of the following genes: NFE2L2, KEAP1, or CUL3; characterizing a subject sample for the presence of a somatic mutation in at least one of the following genes: NFE2L2, KEAP1, or CUL3; determining in a sample (for example, a subject sample or a control sample) a level of a biomarker, for example, AKR1C3; detecting a somatic mutation in one or more gene sequences, for example, an NFE2L2, KEAP1, or CUL3 gene sequence; or detecting a somatic mutation in one or more mRNA sequences, for example, an NFE2L2, KEAP1, or CUL3 mRNA sequence. In some embodiments, a kit described herein includes a pharmaceutical composition comprising a compound of formula (I), or a pharmaceutically acceptable salt thereof. In some embodiments, a kit described herein includes a therapeutically effective amount of a pharmaceutical composition comprising a compound of formula (I), or a pharmaceutically acceptable salt thereof.

In some embodiments, a kit described herein comprises components suitable for determining an mRNA or protein level of a biomarker, for example, AKR1C3. In some embodiments, a kit described herein comprises components suitable for performing an antigen detection assay, for example, an IHC assay. In some embodiments, a kit described herein comprises components suitable for sequencing genomic DNA or RNA (for example, mRNA), for example, an NFE2L2, KEAP1, or CUL3 gene sequence.

### EXAMPLES

### Example 1: An AKRIC3 Immunohistochemistry Assay for Cancer Tissue

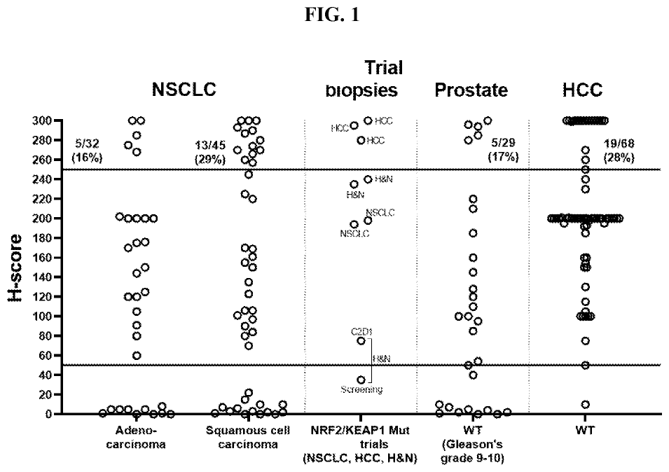

An AKR1C3 immunohistochemistry assay was designed to determine whether detection of AKR1C3 protein expression using an anti-AKR1C3 antibody could be used to identify cancerous tissue. Subject samples from patients diagnosed with NSCLC (including adenocarcinoma and squamous cell carcinoma subtypes; FIG. 1, columns labeled “NSCLC”), prostate cancer (FIG. 1, column labeled “Prostate”), or hepatocellular carcinoma (FIG. 1, column labeled “HCC”), as well as subject sample biopsies of NSCLC, HCC, and head and neck (FIG. 1, H&N) cancer patients (FIG. 1, column labeled “Trial biopsies”) were collected and processed for immunostaining. Prostate group samples were collected from tumors with a Gleason grade score of 9-10 (FIG. 1, “Gleason's grade 9-10”). NSCLC, Prostate, and HCC group subject samples did not harbor mutations detected in NFE2L2 or KEAP1 (FIG. 1, “WT”). Subject samples included in the Trial biopsies group included mutations in NFE2L2 and/or KEAP1 (FIG. 1, “NRF2/KEAP1 Mut”). H&N subject samples included in the Trial biopsies group included biopsies collected prior to treatment (FIG. 1, “Screening”) or collected on cycle 2, day 1 of treatment (FIG. 1, “C2D1”).

All samples were prepared for IHC by cutting 4-6 μm tissue sections from formalin-fixed paraffin-embedded specimen blocks with a microtome. Tissue sections were mounted onto SuperFrost Plus Microscope Slides (Cat. No. 22-037-246; Fisher Scientific, Waltham, MA), air-dried overnight at room temperature, and incubated for 1 hour at 60° C.

Samples included in the Trial biopsies group were stained using a Ventana Benchmark Ultra automated stainer (Ventana, Tucson, AZ), with the following bulk reagents: 10× Reaction buffer (Cat. No. 950-300; Roche Diagnostics Corporation; Indianapolis, IN) diluted to 1× in deionized water, Benchmark Ultra Liquid coverslips (LCS) (Cat. No. 650-210; Roche Diagnostics Corporation), 10× EZ Prep Concentrate (Cat. No. 950-102; Roche Diagnostics Corporation) diluted to 1× in deionized water, and Ultra Cell Conditioning 1 (CC1) Solution (Roche, Cat. No. 950-224). Immunostaining was performed using the ultraView Universal DAB procedure (v1.02.0018) and included antigen retrieval with CC1 Solution for 36 minutes at 95° C. Anti-human AKR1C3 mouse monoclonal primary antibody, clone NP6.G6.A6 (Cat. No. A6229; Sigma Aldrich, St. Louis, MO) was diluted in DAKO Antibody Diluent (Cat. No. S0809; Agilent, Santa Clara, CA), and applied during the primary antibody titration step at a volume of 100 μl. Primary antibody was incubated for 32 minutes at 37° C., and detected with the ultraView Universal DAB detection kit. The slides were counterstained with Hematoxylin (Cat. No. 760-2021; Roche Diagnostics Corporation) for 4 minutes at room temperature and blued with Bluing reagent (Cat. No. 760-2037; Roche Diagnostics Corporation) for 4 minutes at room temperature.

Samples included in the NSCLC, Prostate, and HCC groups were stained using a Ventana Discovery Ultra automated stainer (Ventana, Tucson, AZ), with the following bulk reagents: 10× Reaction buffer (Cat. No. 950-300; Roche Diagnostics Corporation) diluted to 1× in deionized water, Benchmark Ultra Liquid coverslips (LCS) (Cat. No. 650-210; Roche Diagnostics Corporation), Discovery wash concentrate (Cat. No. 950-510; Roche Diagnostics Corporation) diluted to 1× in deionized water, and Ultra Cell Conditioning 1 (CC1) Solution (Roche, Cat. No. 950-224). Immunostaining was performed using the RUO Discovery Universal procedure (v0.00.0370) and included antigen retrieval with CC1 Solution for 32 minutes at 95° C. Endogenous peroxidase was blocked with an 8 minute incubation in Inhibitor CM, a component of the ChromoMap DAB detection kit (Cat. No. 760-159; Roche Diagnostics Corporation). Anti-human AKR1C3 mouse monoclonal primary antibody, clone NP6.G6.A6 (Cat. No. A6229; Sigma Aldrich, St. Louis, MO) was diluted in DAKO Antibody Diluent (Cat. No. S0809; Agilent, Santa Clara, CA), and applied during the primary antibody titration step at a volume of 100 μl. Primary antibody was incubated for 60 minutes at 37° C., followed by a 4 minute incubation with Omnimap anti-Mouse HRP secondary antibody (Cat. No. 760-4310; Roche Diagnostics Corporation), and detection with the ChromoMap DAB detection kit. The slides were counterstained with Hematoxylin (Cat. No. 760-2021; Roche Diagnostics Corporation) for 4 minutes at room temperature and blued with Bluing reagent (Cat. No. 760-2037; Roche Diagnostics Corporation) for 4 minutes at room temperature.

DAB staining of subject samples using anti-AKR1C3 antibody detection and subsequent analysis demonstrated that most subject samples were assigned an H-score above 50 (FIG. 1). Additionally, 5/32 (16%) of NSCLC adenocarcinoma subject samples, 13/45 (29%) of NSCLC squamous cell carcinoma subject samples, 5/29 (17%) of prostate cancer subject samples, and 19/68 (28%) of HCC subject samples were assigned an H-score of 250 or greater. These results demonstrate that an anti-AKR1C3 antibody could be used to detect AKR1C3 expression level and calculate an H-score in subject samples from cancerous tissue.

### Example 2: a Clinical Trial to Determine AKR1C3-Dependent KARS Inhibitor Dosing

A phase I, open-label, multi-center clinical trial study is carried out for the purpose of characterizing the safety, tolerability, and pharmacokinetics of Compound I (6′-fluoro-N-(4-fluorobenzyl)-4′-oxo-3′,4′-dihydro-1′H-spiro[piperidine-4,2′-quinoline]-1-carboxamide) in patients with non-small cell lung cancer. The study is also carried out optionally to identify the maximum tolerated dose and/or recommended dose for Compound I in adult patients with advanced non-small cell lung cancer with or without NFE2L2/KEAP1/CUL3 mutations. The preliminary anti-tumor activity of Compound I is also optionally assessed. The study includes a dose escalation part, followed by a dose expansion part. The escalation portion characterizes the safety and tolerability. The dose expansion portion assesses the preliminary anti-tumor activity in defined patient populations and further assesses the safety and tolerability at MTD/RD.

### Patients and Cohorts

Patients suitable for the study include those with advanced (metastatic or unresectable) non-small cell lung cancer harboring NFE2L2 or KEAP1 or CUL3 mutations (dose escalation and dose expansion group 1) and patients with advanced (metastatic or unresectable) non-small cell lung cancer irrespective of mutational status (dose expansion group 2), for whom standard of care therapy for their indication has failed or who are intolerant of or ineligible for approved therapies.

The dose escalation and dose expansion group 1 includes patients with histologically or cytologically confirmed diagnosis of advanced (metastatic or unresectable) NFE2L2/KEAP1/CUL3 mutant non-small cell lung cancer. Local data confirming the NFE2L2/KEAP1/CUL3 mutation status in tissue is required for enrollment.

The dose expansion group 2 includes patients with histologically or cytologically confirmed diagnosis of advanced (metastatic or unresectable) non-small cell lung cancer irrespective of mutation status.

All patients have progressed after 1 platinum-based chemotherapy regimen and/or PD(L)-1 antibody therapy, where indicated, for Stage IV non-small cell lung cancer. Patients can include those who have undergone prior therapy with VEGF/VEGFR targeting agents, neo-adjuvant/adjuvant therapy. Patients with non-small cell lung cancer whose tumor bears actionable mutations have undergone treatment with approved targeted drugs (for example EGFRi, ALKi, METi). All patients have at least one measurable lesion according to RECIST v1.1. All patients have a site of disease amenable to biopsy and are each a candidate for tumor biopsy according to the treating institution's guidelines. Patients are willing to undergo a new tumor biopsy at screening, and again during therapy on this study. A recent biopsy collected after the last systemic treatment and within 3 months before study entry is optionally submitted at screening.

Patients do not have impaired cardiac function or clinically significant cardiac disease, or risk factors at screening. Patients are not symptomatic for CNS metastases, or CNS metastases that require local CNS-directed therapy (such as radiotherapy or surgery) or increasing doses of corticosteroids 2 weeks prior to study entry. Patients with treated symptomatic brain metastases are neurologically stable (for 4 weeks post-treatment and prior to study entry) and at a dose of ≤10 mg per day prednisone or equivalent for at least 2 weeks before administration of any study treatment. Patients do not include those treated with medications/supplements/herbs that are strong or moderate CYP3A4 inhibitors or strong or moderate CYP3A4 inducers that cannot be discontinued 7 days prior to the start of the study and for the duration of the study.

### Objectives and Endpoints

Efficacy of Compound I treatment is determined on overall response rate, progression-free survival, and duration of response as per RECIST v1.1. Pharmacokinetics are assessed based on plasma concentration vs time profiles and derived pharmacokinetic parameters (e.g., Cmax, Tmax, AUC) for Compound I and its cytotoxic metabolite Compound II ((R)-6′-fluoro-N-(4-fluorobenzyl)-4′-hydroxy-3′,4′-dihydro-1′H-spiro[piperidine-4,2′-quinoline]-1-carboxamide).

The primary objective of the study is to characterize the safety and tolerability of Compound I in patients with NSCLC and identify the MTD(s) and/or RD(s) and dosing regimen for future studies. Safety is determined by the incidence and severity of adverse events (AEs) and serious adverse events (SAEs), including changes in laboratory parameters, vital signs, and electrocardiograms (ECGs). The incidence and nature of dose limiting toxicities (DLTs) is determined during the first 28 days of treatment with Compound I. Tolerability is determined by dose interruptions, reductions, and dose intensity.

Secondary objectives of the study include assessing the preliminary anti-tumor activity of Compound I and evaluating the PK of Compound I. Preliminary anti-tumor activity of Compound I will be determined by overall response rate (ORR), progression-free survival (PFS), and duration of response (DOR) as per RECIST v1.1. Pharmacokinetics of Compound I are determined by analyzing plasma concentration vs time profiles and derived PK parameters for Compound I and Compound II (e.g., Cmax, Tmax, AUC).

### Study Design

Dose escalation. In the dose escalation, a minimum of 21 patients with advanced NSCLC harboring NFE2L2, or KEAP1 or CUL3 (NFE2L2/KEAP1/CUL3) mutations are treated. Patient enrollment is based on locally available test results of mutation status (the same archival sample that was used to determine mutation status locally is optionally requested, if available, for central confirmation).

Cohorts of 3-6 patients are treated with different doses of Compound I orally (p.o.) QD or BID (based on emerging PK data from the dose escalation) on a 28-day cycle until MTD(s) and/or RD(s) is reached.

Safety (including the dose-DLT relationship) and tolerability of Compound I is also assessed, to identify the regimen and/or MTD(s) and/or RD(s) for use in the dose expansion. The dose and regimen for RD is identified after reviewing all available data including PK, safety, and preliminary anti-tumor activity. A Bayesian Hierarchical Logistic Regression Model (BHLRM) using the escalation with overdose control (EWOC) principle guides the dose escalation to determine the MTD(s) and/or RD(s). The RD does not exceed the MTD of Compound I.

Based on emerging PK, safety, tolerability data, and/or preliminary anti-tumor activity, different dosing regimen(s) (e.g., 2 weeks on/2 weeks off, 3 weeks on/1 week off, 1 week on/I week off) are also evaluated.

Dose expansion. The study enters the dose expansion, after an MTD(s) and/or RD(s) is declared in the dose escalation. Approximately 100 patients with advanced NSCLC are treated across two dose expansion groups to assess the preliminary anti-tumor activity of Compound I. At least 10 patients with squamous cell carcinoma are enrolled in each group. The dose expansion groups include the following:
Group 1 (approx. 40 patients): Patients with advanced NSCLC harboring NFE2L2/KEAP1/CUL3 mutations enrolled based on locally available test results of mutation status (the same archival sample that was used to determine mutation status locally is optionally requested, if available, for central confirmation).
Group 2 (approx. 60 patients): Patients with advanced NSCLC irrespective of prior knowledge of NFE2L2/KEAP1/CUL3 mutational status.

The study design is illustrated in FIG. 2.

The study optionally also includes an exploratory assessment of food effect (“FE”) on the PK of Compound I single agent conducted in a separate cohort of patients with advanced NSCLC harboring NFE2L2/KEAP1/CUL3 mutations. For this subset of patients, the study design consists of a FE run-in period and the treatment period.

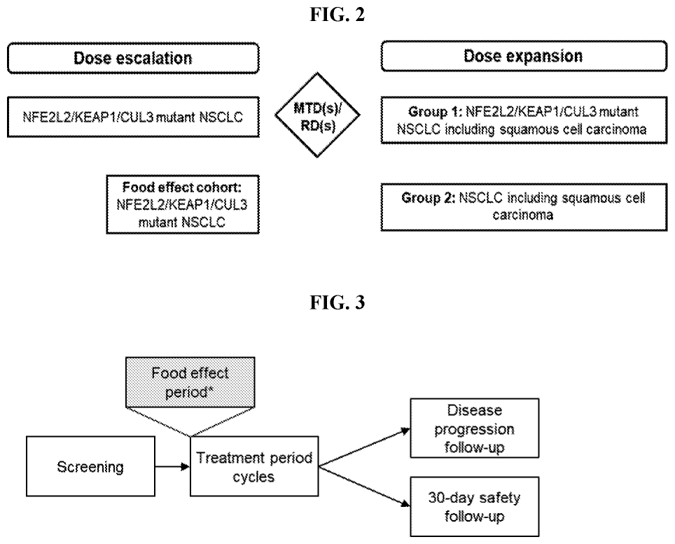

Patients undergo safety and efficacy assessments during screening/baseline and during treatment (FIG. 3).

Compound I is administered orally in the form of capsules of 50 mg or 75 mg drug substance.

The starting dose for Compound I single agent is set at 100 mg, administered p.o. QD on a continuous schedule based on the available preclinical safety, tolerability, and PK/PD data. The selection of the starting dose follows the ICH S9 guidelines for choosing a starting dose for a FIH trial conducted in patients with advanced cancer. The starting dose is also supported by 4-week GLP toxicology studies performed in rats and monkeys.

Table 1 describes the starting dose and possible dose levels evaluated during this trial.

TABLE 1
Provisional dose levels

| | | | Increment from previous |
|---|---|---|---|
| Dose level | Proposed total daily dose* | | dose |
| −1** | 50 | mg | −50% |
| 1 | 100 | mg | (starting dose) |
| 2 | 200 | mg | 100% |
| 3 | 400 | mg | 100% |
| 4 | 600 | mg | 50% |
| 5 | 800 | mg | 33.3% |
| 6 | 1000 | mg | 25% |

*Additional and/or intermediate dose levels are possibly added during the study. Cohorts are optionally added at any dose level below the MTD to better understand safety, PK, or PD. The total daily dose is delivered by QD or BID regimen.
**Dose level −1 represents the dose at which a new cohort is optionally enrolled if, due to observed DLTs, the starting dose is not allowed as an option for the next cohort.

### Biomarkers

Biomarker analyses is used to investigate the effect of Compound I as a single agent at the molecular and cellular level as well as to determine how changes in the markers relate to exposure and clinical outcomes. In addition, potential predictive markers of efficacy, as well as mechanisms of resistance to Compound I as a single agent are optionally explored.

The exact date and time of collection for the biomarker samples is entered on the appropriate eCRF and requisition form(s). Detailed instructions for the collection, processing, and shipment of biomarker samples are provided. Sample(s) are collected at defined visit/time point(s). Biomarker sample type and collection are described in Table 2.

TABLE 2
Biomarker sample collection plan

| | Visit/Time | Approx. | | |
|---|---|---|---|---|
| Sample Type | point | volume | Marker | Purpose |
| Archival tumor | Screening¹ | FFPE Block | Cancer-related genes by | Confirm |
| sample (same | | (preferred) or | next generation DNA | NFE2L2/KEAP1/CUL3 |
| biopsy used for | | 20 newly cut | sequencing (including | alteration status |
| local testing) and | | tissue sections | NFE2L2, KEAP1, CUL3) | Support potential CDx |
| associated | | (4 μm | | assay development |
| pathology report | | thickness) | | |
| Newly obtained | Screening² | 3-6 passes of | Cancer-related genes by | Confirm |
| tumor biopsy | C1D22 | core needle | next generation DNA | NFE2L2/KEAP1/CUL3 |
| | (±3 days, | biopsy | sequencing (including | alterations and biomarker |
| | ideally 2-6 | | NFE2L2, KEAP1, CUL3) | exploration for potential |
| | hours post | | | predictive markers of |
| | dose) | | | response. |
| | | | | Support potential CDx |
| | | | | assay development (in |
| | | | | case archival sample is |
| | | | | not available) |
| | | | Gene expression (e.g., | Potential predictive |
| | | | AKR1C3, KARS, EGR1, | markers of response and |
| | | | ATF3, DDIT3) | pharmacodynamics |
| | | | Protein expression for | |
| | | | AKR1C3 | |
| | | | Whole transcriptome | |
| | | | analysis; expression of | |
| | | | immune and cancer related | |
| | | | genes | |
| | | | Protein expression of | |
| | | | immune-related markers | |
| | | | (e.g., PD-L1, CD8) | |
| Blood sample for | Screening | 10 mL per visit | DNA sequencing in | Assessing cfDNA as |
| cfDNA | EOT | | cfDNA | surrogate for tumor |
| | | | | mutation assessment, and |
| | | | | explore potential |
| | | | | biomarkers of response |
| | | | | and resistance |
| Blood sample for | Screening | 20 mL | Mutation analysis | Potential CDx assay |
| CDx development | | | (e.g., NFE2L2, KEAP1, | development |
| | | | CUL3) | |

¹The same archival sample used to determine the mutation status locally is submitted if available, for central confirmation. In case it is not available, another archival sample is submitted. In each case a copy of a corresponding de-identified pathology report is collected.
²A recent biopsy collected after the last systemic treatment and within 3 months before study entry is optionally submitted at Screening. The sample should meet specifications detailed in the lab manual. In such case, a copy of a corresponding de-identified pathology report is also optionally collected.
Note:
On days and time points when biomarker and pharmacokinetic blood samples are being collected, the PK sample is drawn first.

Newly obtained pre- and on-treatment paired tumor samples are mandatorily collected at screening and on-treatment if safe and medically feasible.

In case a recent biopsy collected after the last systemic treatment and within 3 months before study entry is available, then it is optionally submitted at screening instead of a newly obtained biopsy. In the event an inadequate tumor sample is received at screening following a new biopsy procedure (e.g., found to have low tumor content or insufficient tissue remaining), a recent biopsy is optionally requested to allow for the analysis described in Table 8-13. In both cases, the recent biopsy meet specifications provided and as detailed in the lab manual. A copy of a corresponding de-identified pathology report is also optionally submitted.

When a core needle biopsy is performed, 3-6 tumor biopsy passes are requested at both the screening and post treatment visits. On-treatment biopsy is scheduled within a ±3-day window of C1D22, and ideally 2-6 hours post treatment dosing; the date and time of sampling are recorded in the eCRF. The timing for the on-treatment biopsy (C1D22) is optionally adjusted based on emerging data. Decisions regarding the timing of the on-treatment tumor biopsy are made by investigators.

To the extent possible, tumor biopsies are collected from the same tumor lesion.

The investigator makes a reasonable effort to document the location and size of the lesion where the biopsy was taken (at baseline and on treatment). This information is recorded in the eCRF.

The same archival tumor biopsy used for local testing is submitted at screening if available, to confirm retrospectively the local data related to NFE2L2, KEAP1 and CUL3 alterations, which may support potential companion diagnostics (CDx) assay development. A copy of the corresponding de-identified pathology report is also optionally requested. The mandatory screening biopsy or the recent biopsy collected after the last systemic treatment and within 3 months before study entry is also optionally used to confirm retrospectively the NFE2L2/KEAP1/CUL3 mutation status in a central laboratory (if an archival sample is not available) and for AKR1C3 expression analysis using a central IHC assay. Archival and newly obtained tumor sample are optionally profiled for genetic alterations and optionally support potential CDx assay development.

Collection of newly obtained, paired tumor samples is used to test for the PD effects of Compound I directly in tumor (e.g., expression of ATF3, EGR1, DDIT3, AKR1C3, KARS) and to assess if AKR1C3 and/or KARS expression is a potential predictor of response. Whole transcriptome analysis and expression of additional immune or cancer related genes are also optionally investigated. The protein expression of AKR1C3 is used to support subgroup analysis in dose expansion group 2 and it is optionally tested in dose escalation and dose expansion group 1 as well. Expression and localization of immune biomarkers including but not limited to PD-L1 and CD8 is optionally measured by IHC or using additional techniques deemed suitable.

Blood is collected at screening and at EOT to allow for sequence analysis of cfDNA. This analysis explores the presence of emerging, existing, and resistance mutations and potentially the tumor mutation burden in tumor and cfDNA at different time points and investigate their relationship with clinical response and the development of tumor resistance.

An additional blood sample is collected at screening to support potential CDx assay development for genes including but not limited to NFE2L2, KEAP1 and CUL3.

### AKRIC3 Expression and Antitumor Activity

In order to explore the relationship of baseline AKR1C3 expression on antitumor activity and specifically on ORR according to RECIST v1.1, a logistic regression model is fitted on patient's data from dose expansion group 2. A score (H-Score) is used to quantify the AKR1C3 exposure of the patients.

Assuming that patients with high values of AKR1C3 expression are related to higher probability of response, the estimate of the probability of response according to the fitted model is presented for various AKR1C3 H-Score values (e.g., H-Score=0, 50, 150, 250) together with their 90% confidence intervals. Special attention is paid to the patients with the highest AKR1C3 concentration (H-Score≥250).

An additional analysis is optionally performed to all patients treated in the same dose and whose baseline AKR1C3 expression is available, including all data of patients from the escalation part and dose expansion group 1. Subgroup analyses for specific patient groups (e.g., squamous patients) are optionally considered.

1. A method of identifying a subject for treatment with a compound of formula (I): 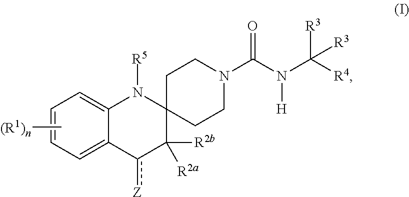
wherein [symbol] is a single bond or a double bond;
Z is either OH, when [symbol] is a single bond; or O, when [symbol] is a double bond;
each R¹ is independently selected from the group consisting of, (C₁-C₆)alkyl, (C₁-C₆)alkoxy, (C₀-C₄)alkylN(R⁸)₂, and halo;
R^{2a} and R^{2b} are each independently selected from the group consisting of H, (C₁-C₆) alkyl, and halo;
each R³ is independently selected from the group consisting of H, and halo;
R⁴ is selected from the group consisting of aryl, a 5 to 6-membered heteroaryl comprising 1, 2, 3, or 4 heteroatoms independently selected from N, O, and S; and a 9 to 10-membered fused bicyclic heteroaryl comprising 1, 2, 3, or 4 heteroatoms independently selected from N, O, and S; wherein any of the foregoing is optionally substituted with one or more R⁶;
R⁵ is selected from the group consisting of H; (C₁-C₆)alkyl; (C₂-C₆)alkenyl; (C₀-C₄)alkylOR⁸; (C₁-C₄)alkyl(C₃-C₁₀)cycloalkyl; halo(C₁-C₆)alkyl; (C₂-C₃)alkynyl; (C₁-C₄)alkylN(R¹⁰)₂;
each R⁶ is independently selected from the group consisting of halo; (C₁-C₆)alkyl; (C₁-C₆)alkoxy; halo(C₁-C₆)alkyl; OH; aryl; 3 to 6-membered heterocycle; 5- to 6-membered heteroaryl; (C₀-C₄)alkylS(O)ₘ(C₁-C₆)alkyl; halo(C₁-C₆)alkoxy; (C₀-C₄)alkylS(O)ₘN(R⁸)₂; (C₀-C₄)alkyl N(R⁸)₂; (C₀-C₄)alkyl(CO)OR⁷; N(R⁸)S(O)ₘ(C₁-C₆)alkyl; N(R⁸)S(O)ₘ(C₃-C₆)cycloalkyl; OP(O)(OH)₂; (C₀-C₃)alkyl(CO)NHR¹¹; (C₀-C₃)alkylOR⁷, and (C₃-C₁₀)cycloalkyl; wherein each R⁶, when not being halo, OH, or OP(O)(OH)₂, is optionally substituted with one to three R⁹; or two neighboring R⁶, together with the atoms to which they attach form a 5 to 7-membered heterocycle or (C₅-C₈)cycloalkyl;
each R⁷ and R⁸ is independently selected from the group consisting of H or (C₁-C₆)alkyl, that is optionally substituted with one to three R⁹;
each R⁹ is independently selected from the group consisting of halo; —OH; amino, (C₁-C₄)alkylamino, di(C₁-C₄)alkylamino, OP(O)(OH)₂; (C₁-C₆)alkyl; (C₁-C₃)alkynyl; (C₁-C₆)alkoxy; halo(C₁-C₆)alkyl; (C₀-C₄)alkylS(O)ₘ(C₁-C₆)alkyl; halo(C₁-C₆)alkoxy; 3 to 6-membered heterocycle which is optionally substituted with oxo (═O); (C₀-C₄)alkylS(O)ₘN(R¹⁰)₂; (C₀-C₄)alkyl(CO)R¹⁰; (C₀-C₄)alkyl(CO)OR¹⁰; (C₀-C₄)alkylNR¹⁰S(O)ₘ(C₁-C₆)alkyl; (C₀-C₄)alkylOR¹⁰; (C₀-C₄)alkylN(R¹⁰)₂; (C₀-C₄)alkylCN; (C₀-C₄)alkylN(R¹⁰)₂; and (C₀-C₄)alkyl(CO)N(R¹⁰)₂;
each R¹⁰ is independently selected from the group consisting of H, (C₁-C₆)alkyl; or 3 to 6-membered heterocycle, wherein the 3 to 6-membered heterocycle is optionally substituted with one or more of (C₁-C₆)alkyl; and oxo (═O);
each R¹¹ is selected from the group consisting of H; 4 to 6-membered heterocycle which is optionally substituted with one to four R¹²; (C₃-C₆)cycloalkyl which is optionally substituted with one to four R¹²; (C₀-C₃)alkyl(C₃-C₆)cycloalkyl (C₁-C₃)alkyl which is optionally substituted with halo; CH₂-aryl which is optionally substituted with one to three R¹²; (C₁-C₆)alkyl; (C₂-C₆)alkenyl; or (C₂-C₆)alkynyl, wherein each of the (C₁-C₆)alkyl; (C₂-C₆)alkenyl; and (C₂-C₆)alkynyl is optionally substituted with one or more R¹³;
each R¹² is independently selected from the group consisting of OH, (C₁-C₃)alkoxy, NH₂; or (C₁-C₃)alkyl optionally substituted with one or more OH;
each R¹³ is independently selected from the group consisting of halo, OH, amino, (C₁-C₄)alkylamino, di(C₁-C₄)alkylamino, (C₁-C₃)alkoxy; and C(O)—(C₃-C₈)cycloalkyl;
m is 0, 1, or 2; and
n is 0, 1 or 2,
or a pharmaceutically acceptable salt thereof,
the method comprising determining in a subject sample a level of AKR1C3, wherein an elevated level of AKR1C3 identifies the subject as a subject in need of treatment with the compound of formula (I), or a pharmaceutically acceptable salt thereof.

2. A method of selecting a compound of formula (I), or a pharmaceutically acceptable salt thereof, for treating a subject, the method comprising determining in a subject sample a level of AKR1C3, wherein an elevated level of AKR1C3 identifies the subject as a subject in need of treatment of the compound of formula (I), or a pharmaceutically acceptable salt thereof.

3. A method of treating a subject, the method comprising:
a. determining in a subject sample a level of AKR1C3, wherein an elevated level of AKR1C3 identifies the subject as in need of treatment with a compound of formula (I), or a pharmaceutically acceptable salt thereof; and
b. administering an effective amount of the compound of formula (I), or a pharmaceutically acceptable salt thereof, to the subject.

4. A method of treating a subject, the method comprising administering to the subject an effective amount of a compound of formula (I), or a pharmaceutically acceptable salt thereof, wherein prior to said administering, a subject sample is characterized as having an elevated level of AKR1C3.

5. A method of treating a subject with a compound of formula (I), or a pharmaceutically acceptable salt thereof, wherein a subject sample is characterized as having an elevated level of AKR1C3.

6. The method of claim 1, wherein the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof.

7. The method of claim 1, wherein the compound of formula (I) is 6′-fluoro-N-(4-fluorobenzyl)-4′-oxo-3′,4′-dihydro-1′H-spiro[piperidine-4,2′-quinoline]-1-carboxamide, or a pharmaceutically acceptable salt thereof.

8. The method of claim 1, wherein the subject sample comprises a cell, a cell population, a cell lysate, a tissue, or a fluid of the subject.

9. The method of claim 8, wherein the cell is a cancerous cell.

10. The method of claim 9, wherein the cancerous cell is a tumor cell.

11. The method of claim 9, wherein the tumor cell is selected from the group consisting of a lung cancer tumor cell, a non-small cell lung cancer tumor cell, a lung adenocarcinoma tumor cell, a lung squamous cell carcinoma cell, a bladder tumor cell, a cervical tumor cell, an esophageal tumor cell, a head and neck tumor cell, a kidney tumor cell, and a liver tumor cell.

12. The method of claim 8, wherein the cell is a lung cell.

13. The method of claim 8, wherein the fluid is selected from the group consisting of blood, plasma, and lymphatic fluid.

14. The method of claim 1, wherein the subject is diagnosed with a disease or disorder selected from the group consisting of a non-small cell lung cancer, a lung adenocarcinoma, a lung squamous cell carcinoma, a bladder cancer, a cervical cancer, an esophageal cancer, a head and neck cancer, a kidney cancer, and a liver cancer.

15. The method of claim 1, wherein the subject tumor genome comprises a somatic mutation in one or more of the NFE2L2, KEAP1, and CUL3 gene sequences.

16. The method of claim 1, wherein the level of AKR1C3 of the subject sample is elevated relative to a control level of AKR1C3.

17. The method of claim 16, wherein the control level comprises a level of AKR1C3 of a control sample or a control data set.

18. The method of claim 17, wherein the control sample comprises a sample selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, and a non-cancerous fluid of a control subject.

19. The method of claim 17, wherein the control data set comprises biomarker level data from a source selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, and a combination thereof.

20. The method of claim 1, wherein the level of AKR1C3 is a protein level of AKR1C3.

21. The method of claim 1, wherein the level of AKR1C3 is an RNA level of AKR1C3.

22. The method of claim 21, wherein the RNA level of AKR1C3 is an mRNA level of AKR1C3.

23-64. (canceled)

65. A method of identifying a subject in need of treatment with a compound of formula (I), or a pharmaceutically acceptable salt thereof, the method comprising detecting in a subject sample a somatic mutation in at least one of the following genes: NFE2L2, KEAP1, or CUL3.

66. A method of selecting a compound of formula (I), or a pharmaceutically acceptable salt thereof, for treating a subject, the method comprising detecting in a subject sample a somatic mutation in at least one of the following genes: NFE2L2, KEAP1, or CUL3.

67. A method of treating a subject, the method comprising:
a. detecting in a subject sample a somatic mutation in at least one of the following genes: NFE2L2, KEAP1, or CUL3, wherein said detecting identifies the subject as in need of treatment with a compound of formula (I), or a pharmaceutically acceptable salt thereof; and
b. administering an effective amount of the compound of formula (I), or a pharmaceutically acceptable salt thereof, to the subject.

68. A method of treating a subject, the method comprising administering to the subject an effective amount of a compound of formula (I), or a pharmaceutically acceptable salt thereof, wherein prior to said administering, a subject sample is characterized by the presence of a somatic mutation in at least one of the following genes: NFE2L2, KEAP1, or CUL3.

69. A method of treating a subject with a compound of formula (I), or a pharmaceutically acceptable salt thereof, wherein a subject sample is characterized by the presence of a somatic mutation in at least one of the following genes: NFE2L2, KEAP1, or CUL3.

70. The method of claim 65, wherein the compound of formula (I) is selected from the group consisting of: 6′-fluoro-N-(4-fluorobenzyl)-4′-oxo-3′,4′-dihydro-1′H-spiro[piperidine-4,2′-quinoline]-1-carboxamide; and N-(4-amino-3-fluorobenzyl)-6′-fluoro-4′-oxo-3′,4′-dihydro-1′H-spiro[piperidine-4,2′-quinoline]-1-carboxamide, or a pharmaceutically acceptable salt thereof.

71. The method of claim 65, wherein the compound of formula (I) is 6′-fluoro-N-(4-fluorobenzyl)-4′-oxo-3′,4′-dihydro-1′H-spiro[piperidine-4,2′-quinoline]-1-carboxamide, or a pharmaceutically acceptable salt thereof.

72. The method of claim 65, wherein the somatic mutation is selected from the group consisting of an amplification of the NFE2L2 gene sequence or a portion thereof, a deletion of the KEAP1 gene sequence or a portion thereof, and a deletion of the CUL3 gene sequence or a portion thereof.

73. The method of claim 65, wherein the somatic mutation comprises a mutation selected from the group consisting of a nonsense mutation, a missense mutation, a substitution mutation, a frameshift mutation, a point mutation, an insertion mutation, a deletion mutation, an inversion mutation, and a gene amplification mutation.

74. The method of claim 65, wherein the somatic mutation comprises a single nucleotide polymorphism (SNP).

75. The method of claim 65, wherein the subject sample comprises a cell, a cell population, a cell lysate, a tissue, or a fluid of the subject.

76. The method of claim 75, wherein the subject sample comprises genomic DNA of the cell, the cell population, the cell lysate, the tissue, or the fluid of the subject.

77. The method of claim 75, wherein the cell is a cancerous cell.

78. The method of claim 77, wherein the cancerous cell is a tumor cell.

79. The method of claim 78, wherein the tumor cell is selected from the group consisting of a lung cancer tumor cell, a non-small cell lung cancer tumor cell, a lung adenocarcinoma tumor cell, a lung squamous cell carcinoma cell, a bladder tumor cell, a cervical tumor cell, an esophageal tumor cell, a head and neck tumor cell, a kidney tumor cell, and a liver tumor cell.

80. The method of claim 75, wherein the cell is a lung cell.

81. The method of claim 75, wherein the fluid is selected from the group consisting of blood, plasma, and lymphatic fluid.

82. The method of claim 65, wherein the subject is diagnosed with a disease or disorder selected from the group consisting of a non-small cell lung cancer, a lung adenocarcinoma, a lung squamous cell carcinoma, a bladder cancer, a cervical cancer, an esophageal cancer, a head and neck cancer, a kidney cancer, and a liver cancer.

83. The method of claim 65, wherein the somatic mutation is absent from a control sample or a control data set.

84. The method of claim 83, wherein the control sample comprises a sample selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, and a non-cancerous fluid of a control subject.

85. The method of claim 84, wherein the control sample comprises genomic DNA of: the non-cancerous cell of the subject, the non-cancerous cell population of the subject, the non-cancerous tissue of the subject, the non-cancerous fluid of the subject, the non-cancerous cell of a control subject, the non-cancerous cell population of a control subject, the non-cancerous tissue of a control subject, or the non-cancerous fluid of a control subject.

86. The method of claim 83, wherein the control data set comprises genomic sequence data from a source selected from the group consisting of a non-cancerous cell of the subject, a non-cancerous cell population of the subject, a non-cancerous tissue of the subject, a non-cancerous fluid of the subject, a non-cancerous cell of a control subject, a non-cancerous cell population of a control subject, a non-cancerous tissue of a control subject, a non-cancerous fluid of a control subject, and a combination thereof.

87-108. (canceled)
